SEQUENCE LISTING (61 sequences)

SEQ ID NO: 1 - DNA, 4215 bp, Artificial Sequence
  Synthetic
cgccccattgacgcaaatgggcggtaggcgtgtacggtgggaggtctatataagcagagctggtttagtgaaccgtcagatcagatctttgtcgatcctaccatccactcgacacacccgccagcggccgctgccaagcttccgagctctcgaattcgccaccatgagccccaagaagaagagaaaggtggaggccagcatcgaaaagaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactggcgcactgggtggtcgctaacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcgcaaacaaggccgtcgccctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattccggatccggacctaagaaaaagaggaaggtggcggccgcttacccatacgatgttccagattacgctgctagcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattgg

SEQ ID NO: 2 - DNA, 4888 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctagaagctttatccacagaatcagggttcggctgcgtcctcgctgctagcatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgaatagctaggatctcggcgatcgtacggccaccatgagccccaagaagaagagaaaggtggaggccagcatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactggcgcactgggtggtcgctaacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcgcaaacaaggccgtcgccctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattccggatccggacctaagaaaaagaggaaggtggcggccgcttacccatacgatgttccagattacgctgctagctaatcgctcagtcgattagcttagcctaccaatttttagtgatatcgagctctacccgtatatttcttcaacagtatgcgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgatactcgagaaggatctaggaacccctagtgatggagttggccactccctctctgcgcgctcgctcgctcactgaggccgggcgaccaaaggtcgcccgacgcccgggctttgcccgggcggcctcagtgagcgagcgagcgcgccag

SEQ ID NO: 3 - DNA, 4260 bp, Artificial Sequence
  Synthetic
cgccccattgacgcaaatgggcggtaggcgtgtacggtgggaggtctatataagcagagctggtttagtgaaccgtcagatcagatctttgtcgatcctaccatccactcgacacacccgccagcggccgctgccaagcttccgagctctcgaattcgccaccatgagccccaagaagaagagaaaggtggaggccaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaacgcatacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatggatccggacctaagaaaaagaggaaggtggcggccgcttacccatacgatgttccagattacgctgctagcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattgg

SEQ ID NO: 4 - DNA, 1818 bp, Artificial Sequence
  Synthetic
cgccccattgacgcaaatgggcggtaggcgtgtacggtgggaggtctatataagcagagctggtttagtgaaccgtcagatcagatctttgtcgatcctaccatccactcgacacacccgccagcggccgcgaacccctaccaactggtcggggtttgaaacgggtcttcgagaagaccccgcggaacattattataacgattgctcgggctgagggaaggactgtcctggggactggaatactaactgatatctcttctttttctttttccccaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaggaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatggcaacaaacttctctctgctgaaacaagccggagatgtcgaagagaatcctggaccggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaaaacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtagggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattgg

SEQ ID NO: 5 - DNA, 1830 bp, Artificial Sequence
  Synthetic
cgccccattgacgcaaatgggcggtaggcgtgtacggtgggaggtctatataagcagagctggtttagtgaaccgtcagatcagatctttgtcgatcctaccatccactcgacacacccgccagcggccgcgaacccctaccaactggtcggggtttgaaaaatgatttgcattatcctgacacaatacagccgcggaacattattataacgattgctcgggctgagggaaggactgtcctggggactggaatactaactgatatctcttctttttctttttccccaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaggaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatggcaacaaacttctctctgctgaaacaagccggagatgtcgaagagaatcctggaccggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaaaacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtagggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattgg

SEQ ID NO: 6 - DNA, 1831 bp, Artificial Sequence
  Synthetic
cgccccattgacgcaaatgggcggtaggcgtgtacggtgggaggtctatataagcagagctggtttagtgaaccgtcagatcagatctttgtcgatcctaccatccactcgacacacccgccagcggccgcgaacccctaccaactggtcggggtttgaaaccaactggcctttgcatctaactacttacaaccgcggaacattattataacgattgctcgggctgagggaaggactgtcctggggactggaatactaactgatatctcttctttttctttttccccaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaggaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatggcaacaaacttctctctgctgaaacaagccggagatgtcgaagagaatcctggaccggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaaaacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtagggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattgg

SEQ ID NO: 7 - DNA, 1831 bp, Artificial Sequence
  Synthetic
cgccccattgacgcaaatgggcggtaggcgtgtacggtgggaggtctatataagcagagctggtttagtgaaccgtcagatcagatctttgtcgatcctaccatccactcgacacacccgccagcggccgcgaacccctaccaactggtcggggtttgaaacgctccctcacatgcccgcctcaacaccataccgcggaacattattataacgattgctcgggctgagggaaggactgtcctggggactggaatactaactgatatctcttctttttctttttccccaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaggaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatggcaacaaacttctctctgctgaaacaagccggagatgtcgaagagaatcctggaccggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaaaacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtagggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattgg

SEQ ID NO: 8 - DNA, 1811 bp, Artificial Sequence
  Synthetic
cgccccattgacgcaaatgggcggtaggcgtgtacggtgggaggtctatataagcagagctggtttagtgaaccgtcagatcagatctttgtcgatcctaccatccactcgacacacccgccagcggccgcgaacccctaccaactggtcggggtttgaaacgggtcttcgagaagaccccgcggaacattattataacgattgctcgggctgagggaaggactgtcctggggactggaatactaactgatatctcttctttttctttttccccaaaacaggctgaagtggcagttccagctgctgtccctgcggcagaggctgaggccgaggtgacgctgcgggagctccaggaagccctggaggaggaggtgctcacccggcagagcctgagccgggagatggaggccatccgcacggacaaccagaacttcgccagtcaactacgcgaggcagaggctcggaaccgggacctagaggcacacgtccggcagttgcaggagcggatggagttgctgcaggcagagggagccacagctgtcacgggggtccccagtccccgggccacggatccaccttcccatctagatggccccccggccgtggctgtgggccagtgcccgctggtggggccaggccccatgcaccgccgccacctgctgctccctgccagggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctcccggttccggagtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaaaacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtagggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattgg

SEQ ID NO: 9 - DNA, 1824 bp, Artificial Sequence
  Synthetic
cgccccattgacgcaaatgggcggtaggcgtgtacggtgggaggtctatataagcagagctggtttagtgaaccgtcagatcagatctttgtcgatcctaccatccactcgacacacccgccagcggccgcgaacccctaccaactggtcggggtttgaaacctggaactgccacttcagcctgtgtatgggccgcggaacattattataacgattgctcgggctgagggaaggactgtcctggggactggaatactaactgatatctcttctttttctttttccccaaaacaggctgaagtggcagttccagctgctgtccctgcggcagaggctgaggccgaggtgacgctgcgggagctccaggaagccctggaggaggaggtgctcacccggcagagcctgagccgggagatggaggccatccgcacggacaaccagaacttcgccagtcaactacgcgaggcagaggctcggaaccgggacctagaggcacacgtccggcagttgcaggagcggatggagttgctgcaggcagagggagccacagctgtcacgggggtccccagtccccgggccacggatccaccttcccatctagatggccccccggccgtggctgtgggccagtgcccgctggtggggccaggccccatgcaccgccgccacctgctgctccctgccagggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctcccggttccggagtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaaaacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtagggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattgg

SEQ ID NO: 10 - DNA, 1914 bp, Artificial Sequence
  Synthetic
cgccccattgacgcaaatgggcggtaggcgtgtacggtgggaggtctatataagcagagctggtttagtgaaccgtcagatcagatctttgtcgatcctaccatccactcgacacacccgccagctccttgcttggtggcatctgtctgaagaaggcacattgtgagttagaagtgatcatgtgagaagactttttaaaccaagggacctgtgacataataaatgtaatacagaactgaaagtccaatgatttgcattatcctgacacaatacagccgcggaacattattataacgattgctcgggctgagggaaggactgtcctggggactggaatactaactgatatctcttctttttctttttccccaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaggaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatggcaacaaacttctctctgctgaaacaagccggagatgtcgaagagaatcctggaccggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaaaacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtagggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattgg

SEQ ID NO: 11 - DNA, 1914 bp, Artificial Sequence
  Synthetic
cgccccattgacgcaaatgggcggtaggcgtgtacggtgggaggtctatataagcagagctggtttagtgaaccgtcagatcagatctttgtcgatcctaccatccactcgacacacccgccagcaatcttactttgagatgaaagctatacctgcatttctgtcataagataaagagggagaaatatcttcccatgtgttgatgatacactttaaaatcacaatccaggagaggcctcaagcaacaactggcctttgcatctaactacttacaaccgcggaacattattataacgattgctcgggctgagggaaggactgtcctggggactggaatactaactgatatctcttctttttctttttccccaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaggaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatggcaacaaacttctctctgctgaaacaagccggagatgtcgaagagaatcctggaccggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaaaacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtagggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattgg

SEQ ID NO: 12 - DNA, 1914 bp, Artificial Sequence
  Synthetic
cgccccattgacgcaaatgggcggtaggcgtgtacggtgggaggtctatataagcagagctggtttagtgaaccgtcagatcagatctttgtcgatcctaccatccactcgacacacccgccagcatgtataagaatggagtaagtatacccatgttcctatcacctccccttgataagtttatcatttgccatattttcttcaaaagttttttgaagaaagaaaagttaacagccaaagcagaagctccctcacatgcccgcctcaacaccataccgcggaacattattataacgattgctcgggctgagggaaggactgtcctggggactggaatactaactgatatctcttctttttctttttccccaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaggaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatggcaacaaacttctctctgctgaaacaagccggagatgtcgaagagaatcctggaccggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaaaacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtagggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattgg

SEQ ID NO: 13 - DNA, 791 bp, Artificial Sequence
  Synthetic
cgccccattgacgcaaatgggcggtaggcgtgtacggtgggaggtctatataagcagagctggtttagtgaaccgtcagatcagatctttgtcgatcctaccatccactcgacacacccgccagcggccgcgaacccctaccaactggtcggggtttgaaacgggtcttcgagaagaccccgcggaacattattataacgattgctcgggctgagggaaggactgtcctggggactggaatactaactgatatctcttctttttctttttccccaaaacaggtccctaggccaggcctatcggaggcgctttccctgctcctgttcgccgttgttctgtctcgtgccgccgccctgggctgcattgggttggtggcccacgccggccaactcaccgcagtctggcgccgcccaggagccgcccgcgctcccggttccggagactacaaagaccatgacggtgattataaagatcatgacatcgactacaaggatgacgatgacaagtaaggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattgg

SEQ ID NO: 14 - DNA, 801 bp, Artificial Sequence
  Synthetic
cgccccattgacgcaaatgggcggtaggcgtgtacggtgggaggtctatataagcagagctggtttagtgaaccgtcagatcagatctttgtcgatcctaccatccactcgacacacccgccagcggccgcgaacccctaccaactggtcggggtttgaaacggcggcacgagacagaacaacggcgaaccgcggaacattattataacgattgctcgggctgagggaaggactgtcctggggactggaatactaactgatatctcttctttttctttttccccaaaacaggtccctaggccaggcctatcggaggcgctttccctgctcctgttcgccgttgttctgtctcgtgccgccgccctgggctgcattgggttggtggcccacgccggccaactcaccgcagtctggcgccgcccaggagccgcccgcgctcccggttccggagactacaaagaccatgacggtgattataaagatcatgacatcgactacaaggatgacgatgacaagtaaggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattgg

SEQ ID NO: 15 - DNA, 1880 bp, Artificial Sequence
  Synthetic
cgccccattgacgcaaatgggcggtaggcgtgtacggtgggaggtctatataagcagagctggtttagtgaaccgtcagatcagatctttgtcgatcctaccatccactcgacacacccgccagcggccgcgaacccctaccaactggtcggggtttgaaacgggtcttcgagaagaccccgcggaacattattataacgattgctcgggctgagggaaggactgtcctggggactggaatactaactgatatctcttctttttctttttccccaaaacaggaccagtccatgggaaattggcagatcaagcgccagaatggagatgatcccttgctgacttaccggttcccaccaaagttcaccctgaaggctgggcaggtggtgacgatctgggctgcaggagctggggccacccacagcccccctaccgacctggtgtggaaggcacagaacacctggggctgcgggaacagcctgcgtacggctctcatcaactccactggggaagaagtggccatgcgcaagctggtgcgctcagtgactgtggttgaggacgacgaggatgaggatggagatgacctgctccatcaccaccacggctcccactgcagcagctcgggggaccccgctgagtacaacctgcgctcgcgcaccgtgctgtgcgggacctgcgggcagcctgccgacaaggcatctgccagcggctcaggagcccaggtgggcggacccatctcctctggctcttctgcctccagtgtcacggtcactcgcagctaccgcagtgtggggggcagtgggggtggcagcttcggggacaatctggtcacccgctcctacctcctgggcaactccagcccccgaacccagagcccccagaactgcagcatcatgggttccggagtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaaaacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtagggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattgg

SEQ ID NO: 16 - DNA, 1891 bp, Artificial Sequence
  Synthetic
cgccccattgacgcaaatgggcggtaggcgtgtacggtgggaggtctatataagcagagctggtttagtgaaccgtcagatcagatctttgtcgatcctaccatccactcgacacacccgccagcggccgcgaacccctaccaactggtcggggtttgaaactcctgccgggaagatagggaagaggaagccgcggaacattattataacgattgctcgggctgagggaaggactgtcctggggactggaatactaactgatatctcttctttttctttttccccaaaacaggaccagtccatgggaaattggcagatcaagcgccagaatggagatgatcccttgctgacttaccggttcccaccaaagttcaccctgaaggctgggcaggtggtgacgatctgggctgcaggagctggggccacccacagcccccctaccgacctggtgtggaaggcacagaacacctggggctgcgggaacagcctgcgtacggctctcatcaactccactggggaagaagtggccatgcgcaagctggtgcgctcagtgactgtggttgaggacgacgaggatgaggatggagatgacctgctccatcaccaccacggctcccactgcagcagctcgggggaccccgctgagtacaacctgcgctcgcgcaccgtgctgtgcgggacctgcgggcagcctgccgacaaggcatctgccagcggctcaggagcccaggtgggcggacccatctcctctggctcttctgcctccagtgtcacggtcactcgcagctaccgcagtgtggggggcagtgggggtggcagcttcggggacaatctggtcacccgctcctacctcctgggcaactccagcccccgaacccagagcccccagaactgcagcatcatgggttccggagtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaaaacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtagggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattgg

SEQ ID NO: 17 - DNA, 935 bp, Artificial Sequence
  Synthetic
cgccccattgacgcaaatgggcggtaggcgtgtacggtgggaggtctatataagcagagctggtttagtgaaccgtcagatcagatctttgtcgatcctaccatccactcgacacacccgccagcggccgcgaacccctaccaactggtcggggtttgaaacgggtcttcgagaagaccccgcggaacattattataacgattgctcgggctgagggaaggactgtcctggggactggaatactaactgatatctcttctttttctttttccccaaaacagggctcccactgcagcagctcgggggaccccgccgagtacaacctgcgctcgcgcaccgtgctgtgcgggacctgcgggcagcctgccgacaaggcatctgccagcggctcaggagcccaggtgggcggacccatctcctctggctcttctgcctccagtgtcacggtcactcgcagctaccgcagtgtggggggcagtgggggtggcagcttcggggacaatctggtcacccgctcctacctcctgggcaactccagcccccgaacccagagcccccagaactgcagcatcatgggttccggagactacaaagaccatgacggtgattataaagatcatgacatcgactacaaggatgacgatgacaagtaaggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattgg

SEQ ID NO: 18 - DNA, 948 bp, Artificial Sequence
  Synthetic
cgccccattgacgcaaatgggcggtaggcgtgtacggtgggaggtctatataagcagagctggtttagtgaaccgtcagatcagatctttgtcgatcctaccatccactcgacacacccgccagcggccgcgaacccctaccaactggtcggggtttgaaacgaaaagatttttggcacggggaggctggggccgcggaacattattataacgattgctcgggctgagggaaggactgtcctggggactggaatactaactgatatctcttctttttctttttccccaaaacagggctcccactgcagcagctcgggggaccccgccgagtacaacctgcgctcgcgcaccgtgctgtgcgggacctgcgggcagcctgccgacaaggcatctgccagcggctcaggagcccaggtgggcggacccatctcctctggctcttctgcctccagtgtcacggtcactcgcagctaccgcagtgtggggggcagtgggggtggcagcttcggggacaatctggtcacccgctcctacctcctgggcaactccagcccccgaacccagagcccccagaactgcagcatcatgggttccggagactacaaagaccatgacggtgattataaagatcatgacatcgactacaaggatgacgatgacaagtaaggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattgg

SEQ ID NO: 19 - DNA, 1888 bp, Artificial Sequence
  Synthetic
cgccccattgacgcaaatgggcggtaggcgtgtacggtgggaggtctatataagcagagctggtttagtgaaccgtcagatcagatctttgtcgatcctaccatccactcgacacacccgccagcggccgcatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaaaacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagggttccggagagaccccgtcccagcggcgcgccacccgcagcggggcgcaggccagctccactccgctgtcgcccacccgcatcacccggctgcaggagaaggaggacctgcaggagctcaatgatcgcttggcggtctacatcgaccgtgtgcgctcgctggaaacggagaacgcagggctgcgccttcgcatcaccgagtctgaagaggtggtcagccgcgaggtgtccggcatcaaggccgcctacgaggccgagctcggggatgcccgcaagacccttgactcagtagccaaggagcgcgcccgcctgcagctggagctgagcaaagtgcgtgaggagtttaaggagctgaaagcgcgcaataccaagaaggagggtgacctgatagctgctcaggctcggctgaaggacctggaggctctgctgaactccaaggaggccgcactgagcactgctctcagtgagaagcgcacgctggagggcgagctgcatgatctgcgcggccaggtggccaaggtaagagaccacatctagcaagctctttctttccatgggttggcctgaccaatagaaactgggcttgtcgagacagagagactcttgcgtttctgataggcacctattggtcttactgaccacccccaaatttgaaaattccgcggaacattattataacgattgctcgggctgagggaaggactgtcctggggactggaaatgtcttcctgggacgaagacaagttgtggaaggtccagttttgaggggctattacaacggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattgg

SEQ ID NO: 20 - DNA, 1887 bp, Artificial Sequence
  Synthetic
cgccccattgacgcaaatgggcggtaggcgtgtacggtgggaggtctatataagcagagctggtttagtgaaccgtcagatcagatctttgtcgatcctaccatccactcgacacacccgccagcggccgcatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaaaacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagggttccggagagaccccgtcccagcggcgcgccacccgcagcggggcgcaggccagctccactccgctgtcgcccacccgcatcacccggctgcaggagaaggaggacctgcaggagctcaatgatcgcttggcggtctacatcgaccgtgtgcgctcgctggaaacggagaacgcagggctgcgccttcgcatcaccgagtctgaagaggtggtcagccgcgaggtgtccggcatcaaggccgcctacgaggccgagctcggggatgcccgcaagacccttgactcagtagccaaggagcgcgcccgcctgcagctggagctgagcaaagtgcgtgaggagtttaaggagctgaaagcgcgcaataccaagaaggagggtgacctgatagctgctcaggctcggctgaaggacctggaggctctgctgaactccaaggaggccgcactgagcactgctctcagtgagaagcgcacgctggagggcgagctgcatgatctgcgcggccaggtggccaaggtaagagaccacatctagcaagctctttctttccatgggttggcctgaccaatagaaactgggcttgtcgagacagagagactcttgcgtttctgataggcacctattggtcttactgaccacccccaaatttgaaaattccgcggaacattattataacgattgctcgggctgagggaaggactgtcctggggactggaaaaaggggtcacagaacacaagagttgtggaaggtccagttttgaggggctattacaacggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattgg

SEQ ID NO: 21 - DNA, 935 bp, Artificial Sequence
  Synthetic
cgccccattgacgcaaatgggcggtaggcgtgtacggtgggaggtctatataagcagagctggtttagtgaaccgtcagatcagatctttgtcgatcctaccatccactcgacacacccgccagcggccgcgaacccctaccaactggtcggggtttgaaacgggtcttcgagaagaccccgcggaacattattataacgattgctcgggctgagggaaggactgtcctggggactggaatactaactgatatctcttctttttctttttccccaaaacagggctctcattgttcaagctcaggggacccggcggaatataacctccgaagccggacggtgctttgtgggacgtgcgggcaaccagcagataaagcgagcgcatccggttctggtgctcaagtcggtggaccaataagcagtggatccagtgctagctcagttacagttacgcgcagctataggagtgtgggtggtagcggtggcggttctttcggcgacaacttggtgactcgaagctacttgctcggaaattccagtccccggacccaaagtccacaaaactgttctataatgggttccggagactacaaagaccatgacggtgattataaagatcatgacatcgactacaaggatgacgatgacaagtaaggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattgg

SEQ ID NO: 22 - DNA, 948 bp, Artificial Sequence
  Synthetic
cgccccattgacgcaaatgggcggtaggcgtgtacggtgggaggtctatataagcagagctggtttagtgaaccgtcagatcagatctttgtcgatcctaccatccactcgacacacccgccagcggccgcgaacccctaccaactggtcggggtttgaaacgaaaagatttttggcacggggaggctggggccgcggaacattattataacgattgctcgggctgagggaaggactgtcctggggactggaatactaactgatatctcttctttttctttttccccaaaacagggctctcattgttcaagctcaggggacccggcggaatataacctccgaagccggacggtgctttgtgggacgtgcgggcaaccagcagataaagcgagcgcatccggttctggtgctcaagtcggtggaccaataagcagtggatccagtgctagctcagttacagttacgcgcagctataggagtgtgggtggtagcggtggcggttctttcggcgacaacttggtgactcgaagctacttgctcggaaattccagtccccggacccaaagtccacaaaactgttctataatgggttccggagactacaaagaccatgacggtgattataaagatcatgacatcgactacaaggatgacgatgacaagtaaggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattgg

SEQ ID NO: 23 - DNA, 150 bp, Artificial Sequence
  Synthetic
tccttgcttggtggcatctgtctgaagaaggcacattgtgagttagaagtgatcatgtgagaagactttttaaaccaagggacctgtgacataataaatgtaatacagaactgaaagtccaatgatttgcattatcctgacacaatacag

SEQ ID NO: 24 - DNA, 150 bp, Artificial Sequence
  Synthetic
aatcttactttgagatgaaagctatacctgcatttctgtcataagataaagagggagaaatatcttcccatgtgttgatgatacactttaaaatcacaatccaggagaggcctcaagcaacaactggcctttgcatctaactacttacaa

SEQ ID NO: 25 - DNA, 150 bp, Artificial Sequence
  Synthetic
atgtataagaatggagtaagtatacccatgttcctatcacctccccttgataagtttatcatttgccatattttcttcaaaagttttttgaagaaagaaaagttaacagccaaagcagaagctccctcacatgcccgcctcaacaccata

SEQ ID NO: 26 - DNA, 30 bp, Artificial Sequence
  Synthetic
aatgatttgcattatcctgacacaatacag

SEQ ID NO: 27 - DNA, 30 bp, Artificial Sequence
  Synthetic
caactggcctttgcatctaactacttacaa

SEQ ID NO: 28 - DNA, 30 bp, Artificial Sequence
  Synthetic
gctccctcacatgcccgcctcaacaccata

SEQ ID NO: 29 - DNA, 30 bp, Artificial Sequence
  Synthetic
ctggaactgccacttcagcctgtgtatggg

SEQ ID NO: 30 - DNA, 30 bp, Artificial Sequence; identical to SEQ ID NO: 29
  Synthetic

SEQ ID NO: 31 - DNA, 28 bp, Artificial Sequence
  Synthetic
tcctgccgggaagatagggaagaggaag

SEQ ID NO: 32 - DNA, 30 bp, Artificial Sequence
  Synthetic
gaaaagatttttggcacggggaggctgggg

SEQ ID NO: 33 - DNA, 22 bp, Artificial Sequence
  Synthetic
aaaggggtcacagaacacaaga

SEQ ID NO: 34 - DNA, 22 bp, Artificial Sequence
  Synthetic
tgcctcgtcccctcagctttca

SEQ ID NO: 35 - DNA, 22 bp, Artificial Sequence
  Synthetic
ggtcaccttcagcttggcggtc

SEQ ID NO: 36 - DNA, 22 bp, Artificial Sequence
  Synthetic
cagcctcgtagtcctgcccaga

SEQ ID NO: 37 - DNA, 22 bp, Artificial Sequence
  Synthetic
aggagaggacaccgttgtgcca

SEQ ID NO: 38 - DNA, 22 bp, Artificial Sequence
  Synthetic
gaaggtgccaccgacacatgca

SEQ ID NO: 39 - DNA, 22 bp, Artificial Sequence
  Synthetic
ggtcaccttcagcttggcggtc

SEQ ID NO: 40 - DNA, 22 bp, Artificial Sequence
  Synthetic
gggtccacctgccttttgtggg

SEQ ID NO: 41 - DNA, 22 bp, Artificial Sequence
  Synthetic
cctgcagcaactccatccgctc

SEQ ID NO: 42 - DNA, 26 bp, Artificial Sequence
  Synthetic
cacggatccaccttcccatctagatg

SEQ ID NO: 43 - DNA, 20 bp, Artificial Sequence
  Synthetic
cagactgcggtgagttggcc

SEQ ID NO: 44 - DNA, 22 bp, Artificial Sequence
  Synthetic
tcaagctggccctggacatgga

SEQ ID NO: 45 - DNA, 22 bp, Artificial Sequence
  Synthetic
tcgatctcgaactcgtggccgt

SEQ ID NO: 46 - DNA, 22 bp, Artificial Sequence
  Synthetic
ctggtgcgctcagtgactgtgg

SEQ ID NO: 47 - DNA, 25 bp, Artificial Sequence
  Synthetic
tggtctttgtagtctccggaaccca

SEQ ID NO: 48 - DNA, 25 bp, Artificial Sequence
  Synthetic
tggtctttgtagtctccggaaccca

SEQ ID NO: 49 - DNA, 22 bp, Artificial Sequence
  Synthetic
gatgctgcagttctgggggctc

SEQ ID NO: 50 - DNA, 22 bp, Artificial Sequence
  Synthetic
ggcgtgctgaagggcgacatta

SEQ ID NO: 51 - DNA, 22 bp, Artificial Sequence
  Synthetic
ccagccggctctcaaactcacg

SEQ ID NO: 52 - protein, 3685 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSDLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 53 - protein, 2527 aa, Homo sapiens
MASGSCQGCEEDEETLKKLIVRLNNVQEGKQIETLVQILEDLLVFTYSERASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 54 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 55 - protein, 664 aa, Homo sapiens
METPSQRRATRSGAQASSTPLSPTRITRLQEKEDLQELNDRLAVYIDRVRSLETENAGLRLRITESEEVVSREVSGIKAAYEAELGDARKTLDSVAKERARLQLELSKVREEFKELKARNTKKEGDLIAAQARLKDLEALLNSKEAALSTALSEKRTLEGELHDLRGQVAKLEAALGEAKKQLQDEMLRRVDAENRLQTMKEELDFQKNIYSEELRETKRRHETRLVEIDNGKQREFESRLADALQELRAQHEDQVEQYKKELEKTYSAKLDNARQSAERNSNLVGAAHEELQQSRIRIDSLSAQLSQLQKQLAAKEAKLRDLEDSLARERDTSRRLLAEKEREMAEMRARMQQQLDEYQELLDIKLALDMEIHAYRKLLEGEEERLRLSPSPTSQRSRGRASSHSSQTQGGGSVTKKRKLESTESRSSFSQHARTSGRVAVEEVDEEGKFVRLRNKSNEDQSMGNWQIKRQNGDDPLLTYRFPPKFTLKAGQVVTIWAAGAGATHSPPTDLVWKAQNTWGCGNSLRTALINSTGEEVAMRKLVRSVTVVEDDEDEDGDDLLHHHHGSHCSSSGDPAEYNLRSRTVLCGTCGQPADKASASGSGAQVGGPISSGSSASSVTVTRSYRSVGGSGGGSFGDNLVTRSYLLGNSSPRTQSPQNCSIM

SEQ ID NO: 56 - protein, 624 aa, Homo sapiens
MSAEVRLRRLQQLVLDPGFLGLEPLLDLLLGVHQELGASELAQDKYVADFLQWAEPIVVRLKEVRLQRDDFEILKVIGRGAFSEVAVVKMKQTGQVYAMKIMNKWDMLKRGEVSCFREERDVLVNGDRRWITQLHFAFQDENYLYLVMEYYVGGDLLTLLSKFGERIPAEMARFYLAEIVMAIDSVHRLGYVHRDIKPDNILLDRCGHIRLADFGSCLKLRADGTVRSLVAVGTPDYLSPEILQAVGGGPGTGSYGPECDWWALGVFAYEMFYGQTPFYADSTAETYGKIVHYKEHLSLPLVDEGVPEEARDFIQRLLCPPETRLGRGGAGDFRTHPFFFGLDWDGLRDSVPPFTPDFEGATDTCNFDLVEDGLTAMETLSDIREGAPLGVHLPFVGYSYSCMALRDSEVPGPTPMELEAEQLLEPHVQAPSLEPSVSPQDETAEVAVPAAVPAAEAEAEVTLRELQEALEEEVLTRQSLSREMEAIRTDNQNFASQLREAEARNRDLEAHVRQLQERMELLQAEGATAVTGVPSPRATDPPSHLDGPPAVAVGQCPLVGPGPMHRRHLLLPARVPRPGLSEALSLLLFAVVLSRAAALGCIGLVAHAGQLTAVWRRPGAARAP

SEQ ID NO: 57 - protein, 7 aa, Artificial Sequence
  Synthetic
  misc_feature (1)..(1): Xaa is a pyrimidine
  misc_feature (3)..(4): Xaa is a pyrimidine
  misc_feature (5)..(5): Xaa is a purine
  misc_feature (7)..(7): Xaa is a pyrimidine
XNXXXAX

SEQ ID NO: 58 - protein, 3 aa, Artificial Sequence
  Synthetic
  misc_feature: Xaa is a pyrimidine
  misc_feature (1)..(1): Xaa can be any naturally occurring amino acid
XAG

SEQ ID NO: 59 - protein, 2 aa, Artificial Sequence
  Synthetic
GT

SEQ ID NO: 60 - protein, 7 aa, Simian virus 40
PKKKRKV

SEQ ID NO: 61 - RNA, 9 nt, Simian virus 40
  misc_feature: where | denotes the exon intron junction
  misc_feature: wherein M = A or C
  misc_feature: wherein R = A or G
magguragu

(19) United States
(12) Patent Application Publication
(10) Pub. No.: US 2024/0124878 A1
(43) Pub. Date: Apr. 18, 2024

(54) COMPOSITIONS FOR AND METHODS OF ENGINEERING THE TRANSCRIPTOME

(71) Applicant: DUKE UNIVERSITY, Durham, NC (US)

(72) Inventors: Aravind Asokan, Durham, NC (US); David Fiflis, Durham, NC (US)

(21) Appl. No.: 18/547,688

(22) PCT Filed: Feb. 25, 2022
(86) PCT No.: PCT/US22/17959

§ 371 Date: Aug. 23, 2023

Related U.S. Application Data
(60) Provisional application No. 63/153,529, filed on Feb. 25, 2021.

(51) Int. Cl. C12N 15/113; C12N 9/22

(52) U.S. Cl. CPC C12N 15/113; C12N 9/22; C12N 2310/20; C12N 2320/33

(57) ABSTRACT

Disclosed herein are compositions for and methods of generating chimeric RNA molecules and methods of treating and/or preventing a genetic disease or disorder using chimeric RNA molecules.

### I. CROSS-REFERENCE TO RELATED APPLICATIONS

This application claims priority to U.S. Provisional Application No. 63/153,529 filed 25 Feb. 2021, which is incorporated herein in its entirety.

### II. STATEMENT REGARDING FEDERAL FUNDING

This invention was made with Government support under Federal Grant No. R01 NS099371 awarded by the National Institute of Neurological Disorders & Stroke (NIH/NINDS). The Federal Government has certain rights to this invention.

### III. REFERENCE TO THE SEQUENCE LISTING

The Sequence Listing submitted 25 Feb. 2022 as a text file named “22_2038_WO_Sequence_Listing”, created on 25 Feb. 2022 and having a size of 142 kilobytes is hereby incorporated by reference pursuant to 37 C.F.R. § 1.52(e)(5).

### IV. BACKGROUND

In eukaryotic organisms, chromosomal DNA is transcribed into precursor RNA messages (pre-mRNA) which contain protein coding regions (exons) and intervening non-protein coding regions (introns). Prior to processing, these pre-mRNA molecules do not possess a sequence primed for translation by the ribosome, due to the retention of non-coding intronic sequences. Thus, prior to nuclear export, the exons of pre-mRNA transcripts are joined through a cellular mechanism known as splicing. This mechanism features dual transesterifications mediated by a large multi ribonucleoprotein structure, called the spliceosome. In the first transesterification, the branch point sequence of the intervening intron attacks the 5′ splice site, forming a lariat structure. This reaction frees the 5′ splice site to attack the 3′ splice site removing the intervening intron, joining the adjacent exons. Upon removal of all intronic sequences, the precursor message matures into a translation competent mature RNA transcript, which is trafficked to the ribosome where it is decoded to manufacture cellular proteins.

In mammalian cells, mutations in transcriptionally active regions of chromosomal DNA give rise to pre-mRNA bearing identical mutations. If the mutation is located in a non-coding region, then processing of the pre-mRNA may be altered or abolished. If the mutation is located in an exonic region of the pre-mRNA, then that mutation will be passed to the mature mRNA sequence. These mutations can contribute to inhibition of complete protein translation of the encoded protein (non-sense mutation) or modify the primary structure of the encoded protein in a counter-productive manner (missense mutation). Collectively, these genetically encoded mutations may function to contribute to pathogenesis in eukaryotes.

The field of gene therapy has aimed to correct such genetic abnormalities through adoptive gene transfer of recombinant nucleic acids bearing a sequence capable of producing the protein product of the mutated gene. This strategy, conventionally termed “classical gene therapy” has proven to be a safe and effective strategy for phenotypic correction of genetic disorders, with several gene therapy products available on the market. However, the standard vector for gene therapy, Adeno-Associated Virus (AAV), has a packaging capacity of ˜4.7 KB. Thus, mutations in genes exceeding this boundary are ineligible targets for gene therapy intervention.

Thus, there remains an urgent need for a minimally invasive, definitive therapy to address the underlying cause of as well as the sequelae of symptoms associated with these various genetic diseases and disorders. Consequently, the present disclosure provides compositions for and methods of generating chimeric RNA molecules and treating and/or preventing a genetic disease and/or disorder, which can be used alone or in combination with other treatments.

### V. BRIEF DESCRIPTION OF THE FIGURES

FIG. 1 is a schematic showing the generation of a 5′ replacement construct to be used in a disclosed method.

FIG. 2 is a schematic showing the replacement strategy for replacing in trans of exons in a 5′ segment of a pre-mRNA using a 5′ replacement construct.

FIG. 3 is a schematic showing the generation of internal replacement constructs to be used in a disclosed method.

FIG. 4 is a schematic showing the replacement strategy for replacing in trans of an internal exon of pre-mRNA using an internal replacement construct.

FIG. 5 is a schematic showing the generation of a 3′ replacement construct to be used in a disclosed method.

FIG. 6 is a schematic showing the replacement strategy for replacing in trans of exons in a 3′ segment of a pre-mRNA using a 3′ replacement construct.

FIG. 7 show the validation of trans-splicing in the DP71 transcript using disclosed composition and methods. Briefly, HEK293 cells were transfected with the following DNA constructs: Lane 1: (SEQ ID NO:01), Lane 2 (SEQ ID NO:07), Lane 3 (SEQ ID NO:01 and SEQ ID NO:04), and Lane 4 (SEQ ID NO:01 and SEQ ID NO:07). 72 hours post-transfection, RNA was harvested with TriZOL reagent using the manufacturer's directions. Purified RNA was converted to cDNA with Applied Biosciences' High Capacity RNA-to-cDNA kit. Trans-splicing was then detected via PCR amplification using primers annealing to exon 72 (SEQ ID NO:34) and the mScarlet ORF (SEQ ID NO:35) of cDNA of cells.

FIG. 8A-FIG. 8B show the 3′ DMD Sanger sequencing confirmation of the transspliced product. FIG. 8A shows a schematic of cis (top) and trans (bottom) spliced RNA products while FIG. 8B shows the alignment of Sanger sequencing traces of cis (top) and trans (bottom) spliced RNA. Notable in the trans-spliced PCR product a silent A>G mutation was observed and highlighted. Briefly, cis-spliced RNA sample corresponds to the same transfection and harvest conditions as Lane 1 of FIG. 7 and trans-spliced sample was gel extracted from the band observed in Lane 4 of FIG. 7. These samples were amplified via PCR with primers comprising the sequence of SEQ ID NO:36 and SEQ ID NO:37.

FIG. 9A-FIG. 9B shows the HTS data for DMD editing using the RNA editing efficiency. FIG. 9A shows the RNA editing strategy with no editing (top) and editing (bottom). FIG. 9B shows that editing efficiency was based on amplicon sequencing of total cDNA from cells amplified with sequencing primer set (SEQ ID NO:36 and SEQ ID NO:37). Efficiency was quantified as the percent of transcripts containing the silent A>G (E3580) mutation. Briefly, HEK293 cells were transfected with the following DNA constructs: Lane 1 (SEQ ID NO:01), Lane 2 (SEQ ID NO:07), Lane 3 (SEQ ID NO:01 and SEQ ID NO:04), Lane 4 (SEQ ID NO:01 and SEQ ID NO:07). 72 hours post-transfection RNA was harvested with TriZOL reagent following manufacturer's directions. Purified RNA was converted to cDNA with Applied Biosciences' High-Capacity RNA-to-cDNA kit and was amplified via PCR amplification using primers SEQ ID NO:36 and SEQ ID NO:37 of cDNA of cells. Amplicons were then processed on an Illumina Hi-Seq and were analyzed using CRISPRESSO2 software.

FIG. 10 shows a comparison of the SMaRT technology vs. Protein Mediated Trans-Splicing HTS. Here, a comparison of RNA trans-splicing via anti-sense targeting based approach in comparison to the proposed RNP-mediated approach via HTS in accordance with one embodiment of the present disclosure. A direct comparison of editing efficiency at the DMD locus was compared between the two approaches to demonstrate the improvement over existing technology. Briefly, 3 separate guides targeting intron 74 of the DMD locus were chosen to compare the system (i.e., Guides A, B, and C). Editing efficiency was based on amplicon sequencing of total cDNA from cells amplified with sequencing primer set (SEQ ID NO:34 and SEQ ID NO:35). Efficiency was quantified as the percent of transcripts containing the silent A>G (E3580) mutation. HEK293 cells were transfected with the following DNA constructs, Guide A/SMaRT (SEQ ID NO:10), Guide A/CRAFT (SEQ ID NO:01 and SEQ ID NO:05), Guide B/SMaRT (SEQ ID NO:11), Guide B/CRAFT (SEQ ID NO:01 and SEQ ID NO:06), Guide C/SMaRT (SEQ ID NO:12), Guide A/CRAFT (SEQ ID NO:01 and SEQ ID NO:07). Purified RNA was converted to cDNA with Applied Biosciences' High-Capacity RNA-to-cDNA kit and amplified via PCR amplification using primers SEQ ID NO:36 and SEQ ID NO:37 of cDNA of cells. Amplicons were then processed on an Illumina Hi-Seq and analyzed using CRISPRESSO2 software.

FIG. 11A-FIG. 11B shows a strategy for 3′ DMPK editing and the subsequent validation of 3′ trans-splicing in the DMPK transcript via binary PCR based readout. Briefly, HEK293 cells were transfected with the following DNA constructs: Lane 1 (SEQ ID NO:01), Lane 2 (SEQ ID NO:09), Lane 3 (SEQ ID NO:01 and SEQ ID NO:08), Lane 4 (SEQ ID NO:01 and SEQ ID NO:09). 72 hours post-transfection RNA was harvested with TriZOL reagent following manufacturer's directions. Purified RNA was converted to cDNA with Applied Biosciences' High-Capacity RNA-to-cDNA kit. Trans-splicing was then detected via PCR amplification using primers annealing to DMPK exon 7 (SEQ ID NO:38) and the mScarlet ORF (SEQ ID NO:39) of cDNA of cells. FIG. 11B shows that precise amplification of target DNA yielded a band at ˜1 kb as observed exclusively in Lane 4 of the gel.

FIG. 12 shows the 3′ DMPK Sanger sequencing results, which confirmed the trans-spliced product. Alignment of the cDNA obtained from wild HEK293 cells against the trans-spliced PCR product from the lane 4 of FIG. 11. Alignment of sanger sequencing traces of cis (top) and trans (bottom) spliced RNA. Notable in the trans-spliced PCR product a silent G>T mutation was observed and highlighted. Briefly, cis-spliced RNA sample corresponded to the same transfection and harvest conditions as Lane 1 of FIG. 12, and trans-spliced sample was gel extracted from the band observed in Lane 4 of FIG. 1i. These samples were amplified via PCR with primers comprising the sequence of SEQ ID NO:40 and SEQ ID NO:41.

FIG. 13A-FIG. 13B show the validation of 3′ trans-splicing in the LMNA transcript via binary PCR based readout. Briefly, HEK293 cells were transfected with the following DNA constructs: Lane 1 (SEQ ID NO: 16), Lane 2 (SEQ ID NO:01 and SEQ ID NO:15), Lane 3 (SEQ ID NO:01 and SEQ ID NO:16). 72 hours post-transfection, RNA was harvested with TriZOL reagent following manufacturer's directions. Purified RNA was converted to cDNA with Applied Biosciences' High-Capacity RNA-to-cDNA kit. Trans-splicing was then detected via PCR amplification using primers annealing to LMNA exon 6 (SEQ ID NO:44) and the mScarlet ORF (SEQ ID NO:45) of cDNA of cells. Precise amplification of target DNA yielded a band at ˜1 kb as observed exclusively in Lane 3 of the gel (FIG. 13B).

FIG. 14 shows 3′ LMNA Sanger sequencing confirmation of the trans-spliced product. Alignment of the cDNA obtained from wild HEK293 cells (top) against the trans-spliced PCR product (bottom) from the lane 3 of FIG. 13. Notable in the trans-spliced PCR product a silent G>A mutation was observed and is highlighted.

FIG. 15 shows 3′ LMNA codon optimized replacement, which demonstrated the complete rewriting of replaced DNA sequence. Briefly, HEK293 cells were transfected with SEQ ID NO:01 and SEQ ID NO:22. Then, 72 hours post-transfection, RNA was harvested with TriZOL reagent following the manufacturer's directions. Purified RNA was converted to cDNA with Applied Biosciences' High-Capacity RNA-to-cDNA kit. Trans-splicing was then detected via PCR amplification using primers annealing to LMNA exon 10 (SEQ ID NO:46) and the 3′ UTR (SEQ ID NO:47) of cDNA of cells. Sanger sequencing was completed with a primer corresponding to SEQ ID NO:46. At the top is a schematic showing the trans-spliced RNA molecule generated comprising the endogenous exons 1-10 of human LMNA, followed by codon optimized exons 11-12 of human LMNA. Below is an alignment of the codon optimized sequence to the hg38 reference transcript, and the exon 10-11 exon junction is denoted. Notably the alignment to exon 10 is perfect, whereas the alignment to exon 11 displays a difference at the codon optimized sequence. A representative Sanger sequencing trace is shown.

FIG. 16 shows 5′ LMNA editing gel, validating the 5′ trans-splicing in the LMNA transcript via binary PCR based readout. Briefly, HEK293 cells were transfected with the following DNA constructs: Lane 1 (SEQ ID NO:03), Lane 2 (SEQ ID NO:20), Lane 3 (SEQ ID NO:03 and SEQ ID NO:19), and Lane 4 (SEQ ID N0:03 and SEQ ID NO:20). Then, 72 hours pos-transfection, RNA was harvested with TriZOL reagent following manufacturer's directions. Purified RNA was converted to cDNA with Applied Biosciences' High-Capacity RNA-to-cDNA kit. Trans-splicing was then detected via PCR amplification using primers annealing to LMNA exon 4 (SEQ ID NO:50) and the mScarlet ORF (SEQ ID NO:51) of cDNA of cells. Precise amplification of target DNA yielded a band at ˜1 kb as observed exclusively in Lane 4 of the gel.

FIG. 17 shows 5′ LMNA Sanger sequencing, which confirmed the trans-spliced product. Alignment of the cDNA obtained from wild HEK293 cells (top) against the trans-spliced PCR product from the lane 4 of FIG. 16. Notable in the trans-spliced PCR product, a silent G>C mutation was observed and is highlighted. Briefly, cis-spliced RNA sample corresponded to the same transfection and harvest conditions as Lane 1 of FIG. 16, and trans-spliced sample was gel extracted from the band observed in Lane 4 of FIG. 16. The primer corresponding to SEQ ID NO. 51 was used to sequence these samples.

FIG. 18 provide the RNA editing strategy and HTS data for DMPK editing. The editing efficiency was based on amplicon sequencing of total cDNA from cells amplified with sequencing primer set (SEQ ID NO:40 and SEQ ID NO:41). Efficiency was quantified as the percent of transcripts containing the silent T>A (P593) mutation. Briefly, HEK293 cells were transfected with the following DNA constructs: Lane 1 (SEQ ID NO:01), Lane 2 (SEQ ID NO:14), Lane 3 (SEQ ID NO:01 and SEQ ID NO:13), and Lane 4 (SEQ ID NO:01 and SEQ ID NO:14). Then, 72 hours post-transfection, RNA was harvested with TriZOL reagent following the manufacturer's directions. Purified RNA was converted to cDNA with Applied Biosciences' High-Capacity RNA-to-cDNA kit and amplified via PCR amplification using primers SEQ ID NO:42 and SEQ ID NO:43 of cDNA of cells. Amplicons were then processed on an Illumina Hi-Seq and was analyzed using CRISPRESSO2 software.

FIG. 19 provides the RNA editing strategy and HTS data for LMNA editing. The editing efficiency was based on amplicon sequencing of total cDNA from cells amplified with sequencing primer set (SEQ ID NO:48 and SEQ ID NO:49). Efficiency was quantified as the percent of transcripts containing the silent T>C (A577) mutation. Briefly, HEK293 cells were transfected with the following DNA constructs: Lane 1 (SEQ ID NO:01), Lane 2 (SEQ ID NO:18), Lane 3 (SEQ ID NO:01 and SEQ ID NO:17), and Lane 4 (SEQ ID NO:01 and SEQ ID NO:18). Then, 72 hours post-transfection, RNA was harvested with TriZOL reagent following manufacturer's directions. Purified RNA was converted to cDNA with Applied Biosciences' High-Capacity RNA-to-cDNA kit and was amplified via PCR amplification using primers SEQ ID NO:48 and SEQ ID NO:49 of cDNA of cells. Amplicons were then processed on an Illumina Hi-Seq and was analyzed using CRISPRESSO2 software.

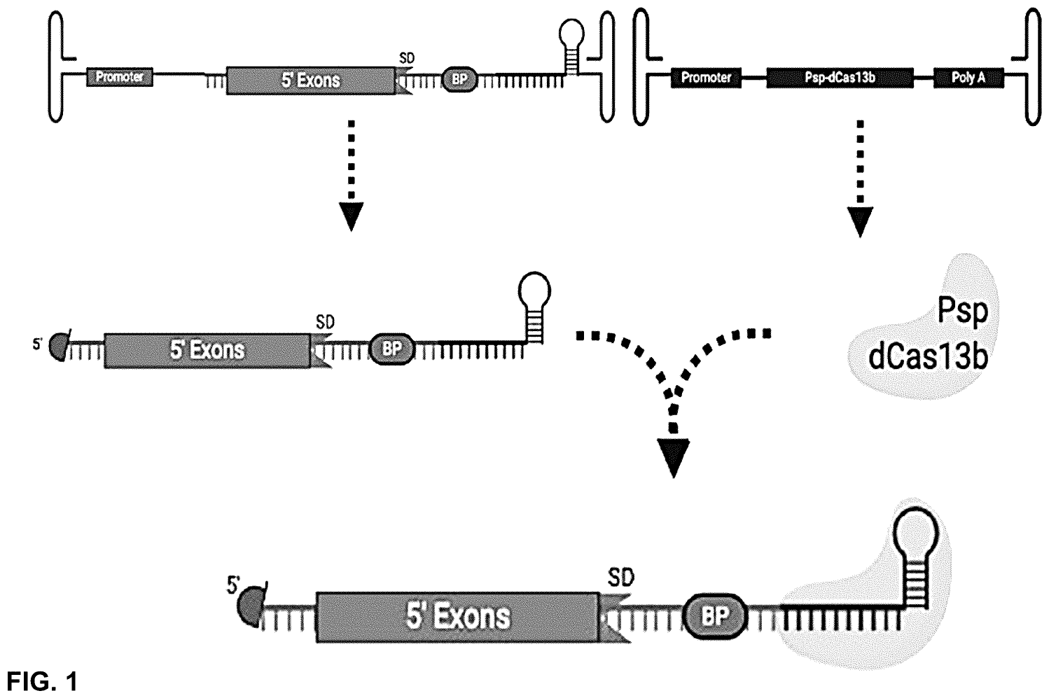

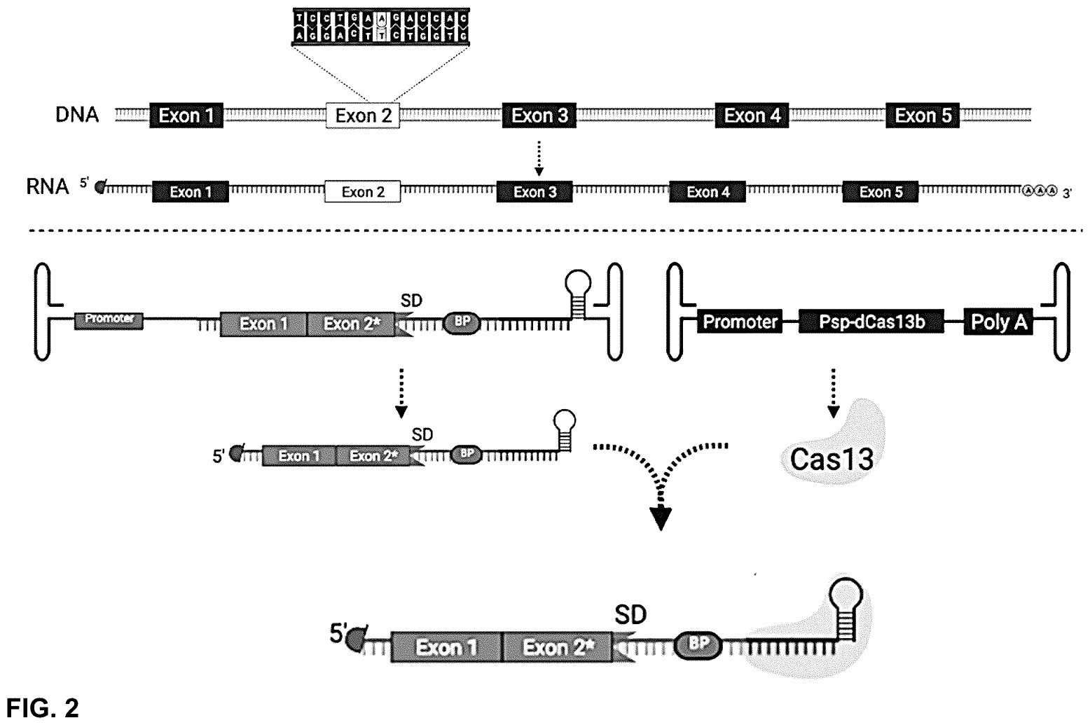

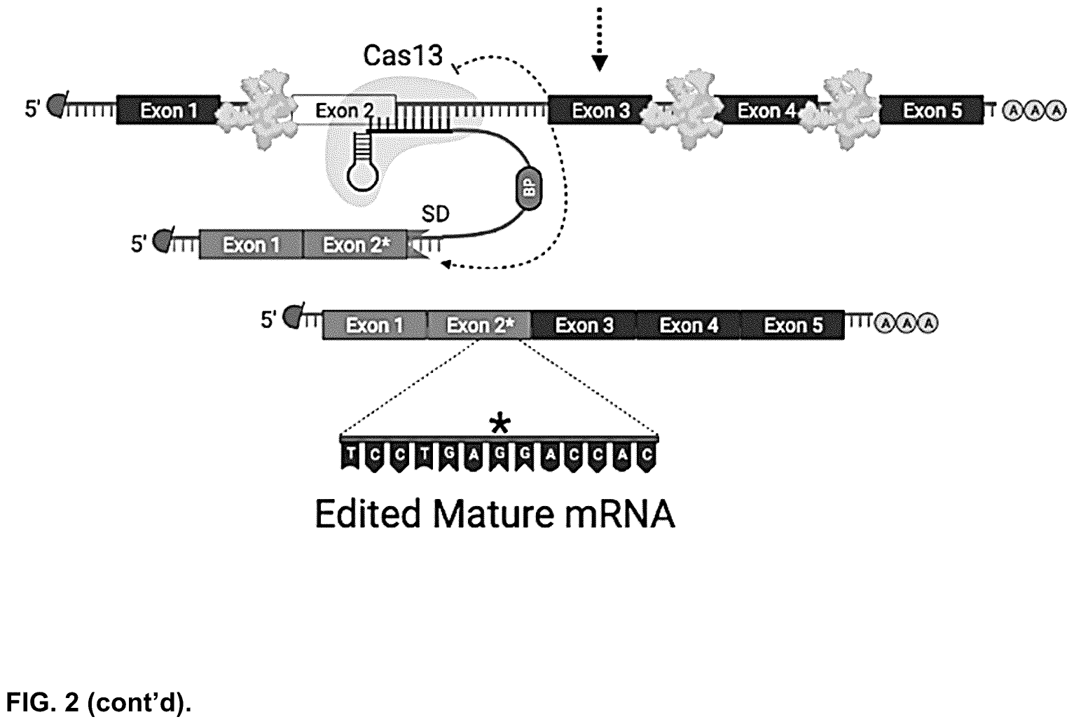

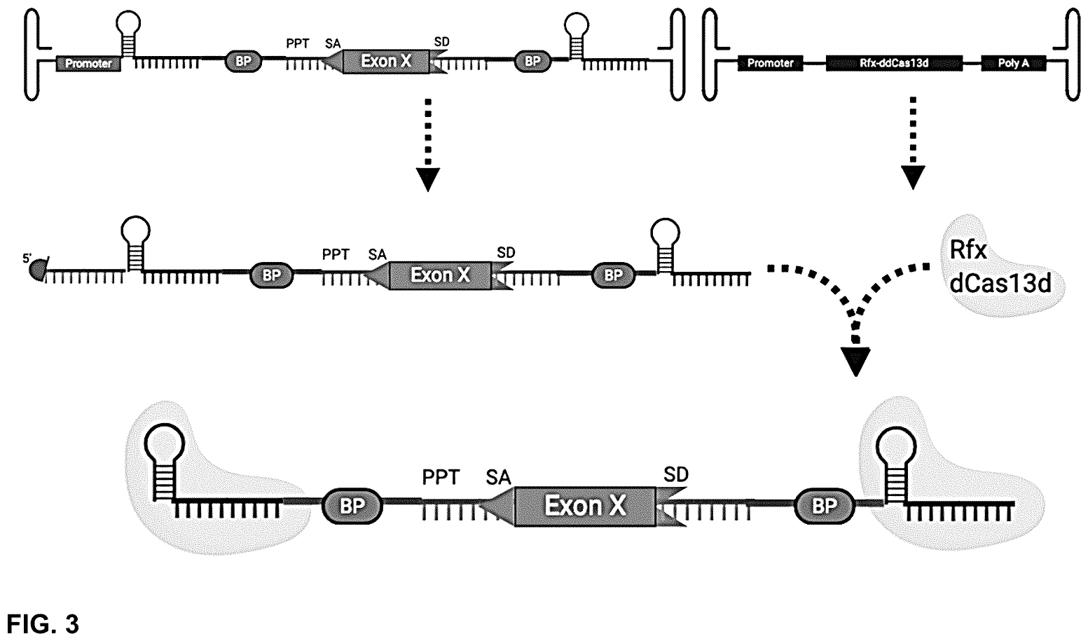

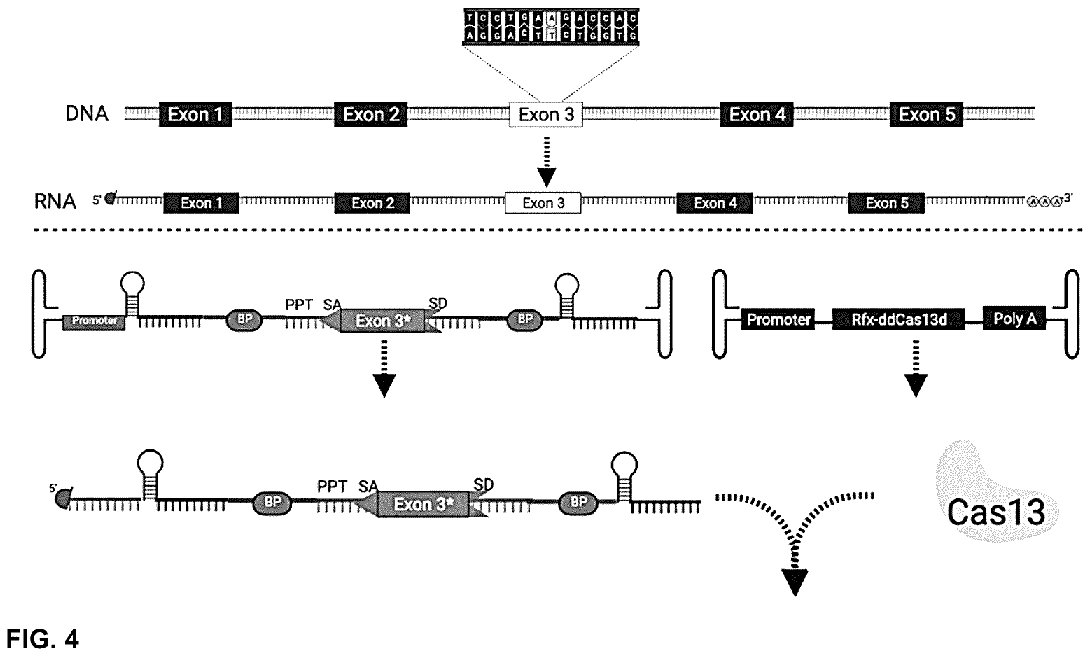

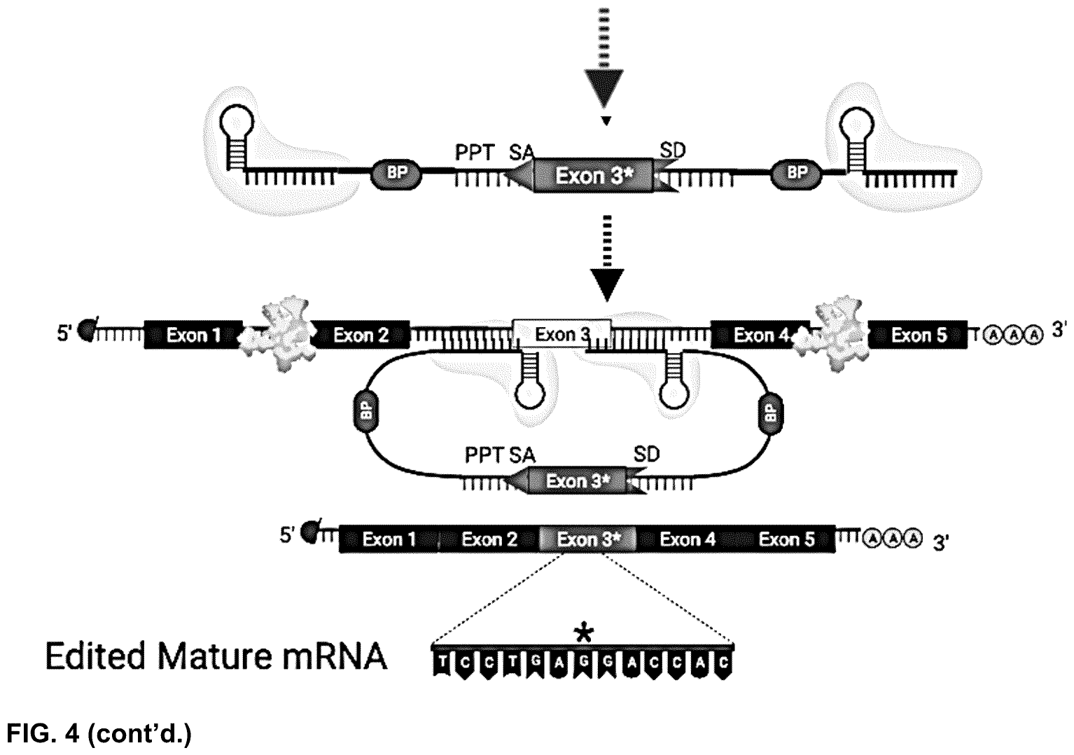

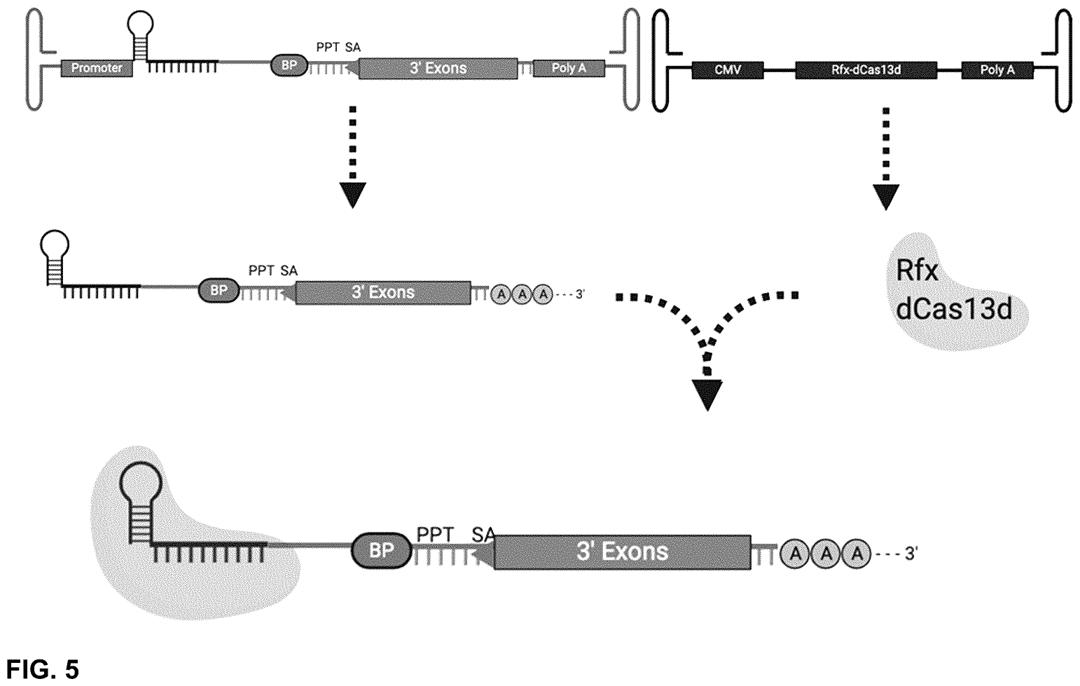

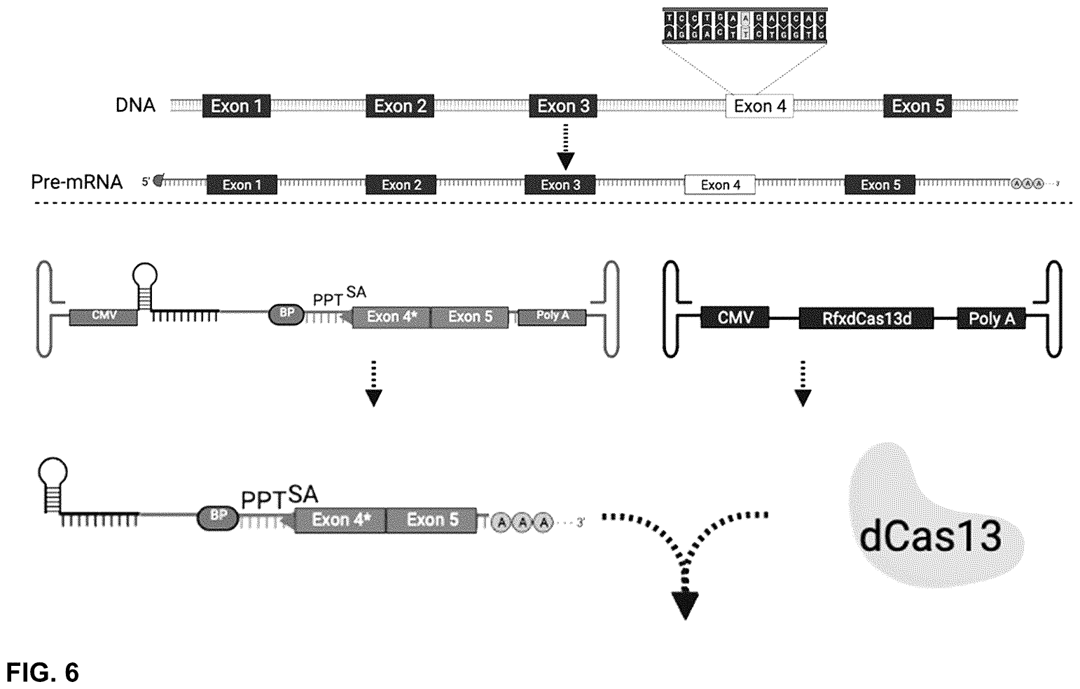

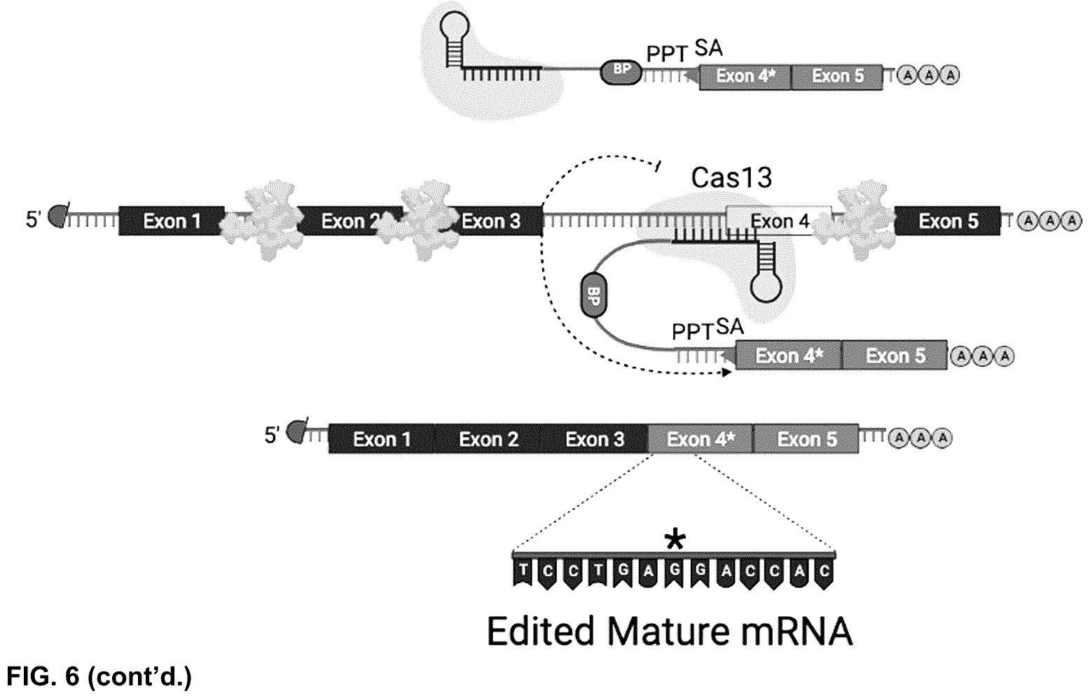

### VI. BRIEF SUMMARY

Disclosed herein is an isolated nucleic acid molecule comprising a nucleic acid sequence to be trans-spliced to a target endogenous pre-mRNA; a 5′ hemi intron linked to the nucleic acid sequence to be trans-spliced; one or more guide RNA sequences; a promoter operably linked to the one or more guide RNA sequences; and a nucleic acid sequence encoding an RNA binding protein.

Disclosed herein is an isolated nucleic acid molecule, comprising a nucleic acid sequence encoding a catalytically inactive PspdCas13b, a promoter operably linked to the nucleic acid sequence encoding the catalytically inactive PspdCas13b; and a polyadenylation signal.

Disclosed herein is an isolated nucleic acid molecule comprising a nucleic acid sequence to be trans-spliced to a target endogenous pre-mRNA; a 3′ hemi intron; a 5′ hemi intron; two or more guide RNA sequences; a promoter operably linked to the two or more guide RNA sequences; and a nucleic acid sequence encoding one or more RNA binding proteins.

Disclosed herein is an isolated nucleic acid molecule, comprising a nucleic acid sequence encoding a catalytically inactive RfxCas13d; a promoter operably linked to the nucleic acid sequence encoding the catalytically inactive RfxCas13d; and a polyadenylation signal.

Disclosed herein is an isolated nucleic acid molecule comprising a nucleic acid sequence to be trans-spliced to a target endogenous pre-mRNA; a 3′ hemi intron linked to the nucleic acid sequence to be trans-spliced; one or more guide RNA sequences; a promoter operably linked to the one or more guide RNA sequences; and a nucleic acid sequence encoding an RNA binding protein.

Disclosed herein is an isolated nucleic acid molecule, comprising a nucleic acid sequence encoding a catalytically inactive RfxCas13d; a promoter operably linked to the nucleic acid sequence encoding the catalytically inactive RfxCas13d; and a polyadenylation signal.

Disclosed herein is a transcriptome engineering system comprising (i) a first isolated nucleic acid molecule comprising a nucleic acid sequence to be trans-spliced to a target endogenous pre-mRNA; a 3′ hemi intron linked to the nucleic acid sequence to be trans-spliced; one or more guide RNA sequences; a promoter operably linked to the one or more guide RNA sequences; and a nucleic acid sequence encoding an RNA binding protein; and (ii) a second isolated nucleic acid molecule comprising a nucleic acid sequence encoding a catalytically inactive RfxCas13d, a promoter operably linked to the nucleic acid sequence encoding the catalytically inactive RfxCas13d; and a polyadenylation signal.

In an aspect, a disclosed first isolated nucleic acid molecule and a disclosed second isolated nucleic acid molecule can form a ternary complex with the endogenous pre-mRNA molecule. In an aspect, a disclosed resulting chimeric RNA molecule can comprise the trans-spliced nucleic acid sequence.

Disclosed herein is a transcriptome engineering system comprising (i) a first isolated nucleic acid molecule comprising a nucleic acid sequence to be trans-spliced to a target endogenous pre-mRNA, a 5′ hemi intron linked to the nucleic acid sequence to be trans-spliced; one or more guide RNA sequences; a promoter operably linked to the one or more guide RNA sequences; and a nucleic acid sequence encoding an RNA binding protein; and (ii) a second isolated nucleic acid molecule comprising a nucleic acid sequence encoding a catalytically inactive PspdCas13b, a promoter operably linked to the nucleic acid sequence encoding the catalytically inactive PspdCas13b; and a polyadenylation signal.

Disclosed herein is a transcriptome engineering system, comprising (i) a first isolated nucleic acid molecule comprising a nucleic acid sequence to be trans-spliced to a target endogenous pre-mRNA; a 3′ hemi intron; a 5′ hemi intron; two or more guide RNA sequences; a promoter operably linked to the two or more guide RNA sequences; and a nucleic acid sequence encoding one or more RNA binding proteins; and (ii) a second isolated nucleic acid molecule comprising a nucleic acid sequence encoding a catalytically inactive RfxCas13d, a promoter operably linked to the nucleic acid sequence encoding the catalytically inactive RfxCas13d; and a polyadenylation signal.

Disclosed herein is a vector comprising a disclosed isolated nucleic acid molecule. Disclosed herein is a vector comprising one or more disclosed isolated nucleic acid molecules.

Disclosed herein is a vector comprising an isolated nucleic acid molecule comprising a nucleic acid sequence to be trans-spliced to a target endogenous pre-mRNA; a 5′ hemi intron linked to the nucleic acid sequence to be trans-spliced; one or more guide RNA sequences; a promoter operably linked to the one or more guide RNA sequences; and a nucleic acid sequence encoding an RNA binding protein.

Disclosed herein is a vector comprising an isolated nucleic acid molecule comprising a nucleic acid sequence to be trans-spliced to a target endogenous pre-mRNA; a 3′ hemi intron; a 5′ hemi intron; two or more guide RNA sequences; a promoter operably linked to the two or more guide RNA sequences; and a nucleic acid sequence encoding one or more RNA binding proteins.

Disclosed herein is a vector comprising an isolated nucleic acid molecule comprising a nucleic acid sequence to be trans-spliced to a target endogenous pre-mRNA; a 3′ hemi intron linked to the nucleic acid sequence to be trans-spliced; one or more guide RNA sequences; a promoter operably linked to the one or more guide RNA sequences; and a nucleic acid sequence encoding an RNA binding protein.

Disclosed herein is a vector comprising an isolated nucleic acid molecule comprising a nucleic acid sequence encoding a catalytically inactive RfxCas13d; a promoter operably linked to the nucleic acid sequence encoding the catalytically inactive RfxCas13d; and a poly adenylation signal.

Disclosed herein is a vector comprising an isolated nucleic acid molecule comprising a nucleic acid sequence encoding a catalytically inactive PspdCas13b, a promoter operably linked to the nucleic acid sequence encoding the catalytically inactive PspdCas13b; and a polyadenylation signal.

Disclosed herein is a pharmaceutical formulation comprising a vector comprising an isolated nucleic acid molecule comprising a nucleic acid sequence to be trans-spliced to a target endogenous pre-mRNA; a 5′ hemi intron linked to the nucleic acid sequence to be trans-spliced; one or more guide RNA sequences; a promoter operably linked to the one or more guide RNA sequences; and a nucleic acid sequence encoding an RNA binding protein.

Disclosed herein is a pharmaceutical formulation comprising a vector comprising an isolated nucleic acid molecule comprising a nucleic acid sequence to be trans-spliced to a target endogenous pre-mRNA; a 3′ hemi intron; a 5′ hemi intron; two or more guide RNA sequences; a promoter operably linked to the two or more guide RNA sequences; and a nucleic acid sequence encoding one or more RNA binding proteins.

Disclosed herein is a pharmaceutical formulation comprising a vector comprising an isolated nucleic acid molecule comprising a nucleic acid sequence to be trans-spliced to a target endogenous pre-mRNA; a 3′ hemi intron linked to the nucleic acid sequence to be trans-spliced; one or more guide RNA sequences; a promoter operably linked to the one or more guide RNA sequences; and a nucleic acid sequence encoding an RNA binding protein.

Disclosed herein is a pharmaceutical formulation comprising a vector comprising an isolated nucleic acid molecule comprising a nucleic acid sequence encoding a catalytically inactive RfxCas13d; a promoter operably linked to the nucleic acid sequence encoding the catalytically inactive RfxCas13d; and a polyadenylation signal.

Disclosed herein is a pharmaceutical formulation comprising a vector comprising an isolated nucleic acid molecule comprising a nucleic acid sequence encoding a catalytically inactive PspdCas13b, a promoter operably linked to the nucleic acid sequence encoding the catalytically inactive PspdCas13b; and a polyadenylation signal.

Disclosed herein is a method of generating a chimeric RNA molecule in a cell, the method comprising contacting an endogenous pre-mRNA in a cell with (i) an isolated nucleic acid molecule comprising a nucleic acid sequence to be trans-spliced to a target endogenous pre-mRNA; a 5′ hemi intron linked to the nucleic acid sequence to be trans-spliced; one or more guide RNA sequences; a promoter operably linked to the one or more guide RNA sequences; and a nucleic acid sequence encoding an RNA binding protein, and (ii) an isolated nucleic acid molecule, comprising a nucleic acid sequence encoding a catalytically inactive PspdCas13b, a promoter operably linked to the nucleic acid sequence encoding the catalytically inactive PspdCas13b; and a polyadenylation signal, wherein the isolated nucleic acid molecules form a ternary complex with the endogenous pre-mRNA molecule, and wherein the resulting chimeric RNA molecule comprises the trans-spliced nucleic acid sequence.

Disclosed herein is a method of generating a chimeric RNA molecule in a cell, the method comprising contacting an endogenous pre-mRNA in a cell with (i) an isolated nucleic acid molecule comprising a nucleic acid sequence to be trans-spliced to a target endogenous pre-mRNA; a 3′ hemi intron; a 5′ hemi intron; two or more guide RNA sequences; a promoter operably linked to the two or more guide RNA sequences; and a nucleic acid sequence encoding one or more RNA binding proteins; and (ii) an isolated nucleic acid molecule, comprising a nucleic acid sequence encoding a catalytically inactive RfxCas13d; a promoter operably linked to the nucleic acid sequence encoding the catalytically inactive RfxCas13d; and a polyadenylation signal, wherein the isolated nucleic acid molecules form a ternary complex with the endogenous pre-mRNA molecule, and wherein the resulting chimeric RNA molecule comprises the trans-spliced nucleic acid sequence.

Disclosed herein is a method of generating a chimeric RNA molecule in a cell, the method comprising contacting an endogenous pre-mRNA in a cell with (i) an isolated nucleic acid molecule comprising a nucleic acid sequence to be trans-spliced to a target endogenous pre-mRNA; a 3′ hemi intron linked to the nucleic acid sequence to be trans-spliced; one or more guide RNA sequences; a promoter operably linked to the one or more guide RNA sequences; and a nucleic acid sequence encoding an RNA binding protein; and (ii) an isolated nucleic acid molecule, comprising a nucleic acid sequence encoding a catalytically inactive RfxCas13d; a promoter operably linked to the nucleic acid sequence encoding the catalytically inactive RfxCas13d; and a polyadenylation signal, wherein the isolated nucleic acid molecules form a ternary complex with the endogenous pre-mRNA molecule, and wherein the resulting chimeric RNA molecule comprises the trans-spliced nucleic acid sequence.

Disclosed herein is a method of treating and/or preventing a genetic disease or disorder, the method comprising generating a chimeric RNA molecule in one or more cells by administering to a subject in need thereof (i) an isolated nucleic acid molecule comprising a nucleic acid sequence to be trans-spliced to a target endogenous pre-mRNA; a 5′ hemi intron linked to the nucleic acid sequence to be trans-spliced; one or more guide RNA sequences; a promoter operably linked to the one or more guide RNA sequences; and a nucleic acid sequence encoding an RNA binding protein, and (ii) an isolated nucleic acid molecule comprising a nucleic acid sequence encoding a catalytically inactive PspdCas13b, a promoter operably linked to the nucleic acid sequence encoding the catalytically inactive PspdCas13b; and a polyadenylation signal, wherein the resulting chimeric RNA molecule comprises the trans-spliced nucleic acid sequence; and wherein the resulting chimeric RNA molecule can restore one or more aspects of cellular homeostasis and/or cellular functionality and/or metabolic dysregulation.

Disclosed herein is a method of treating and/or preventing a genetic disease or disorder, the method comprising generating a chimeric RNA molecule in one or more cells by administering to a subject in need thereof (i) an isolated nucleic acid molecule comprising a nucleic acid sequence to be trans-spliced to a target endogenous pre-mRNA; a 3′ hemi intron; a 5′ hemi intron; two or more guide RNA sequences; a promoter operably linked to the two or more guide RNA sequences; and a nucleic acid sequence encoding one or more RNA binding proteins; and (ii) an isolated nucleic acid molecule comprising a nucleic acid sequence encoding a catalytically inactive RfxCas13d; a promoter operably linked to the nucleic acid sequence encoding the catalytically inactive RfxCas13d; and a polyadenylation signal, wherein the resulting chimeric RNA molecule comprises the trans-spliced nucleic acid sequence; and wherein the resulting chimeric RNA molecule can restore one or more aspects of cellular homeostasis and/or cellular functionality and/or metabolic dysregulation.

Disclosed herein is a method of treating and/or preventing a genetic disease or disorder, the method comprising generating a chimeric RNA molecule in one or more cells by administering to a subject in need thereof (i) an isolated nucleic acid molecule comprising a nucleic acid sequence to be trans-spliced to a target endogenous pre-mRNA; a 3′ hemi intron linked to the nucleic acid sequence to be trans-spliced; one or more guide RNA sequences; a promoter operably linked to the one or more guide RNA sequences; and a nucleic acid sequence encoding an RNA binding protein; and (ii) an isolated nucleic acid molecule, comprising a nucleic acid sequence encoding a catalytically inactive RfxCas13d; a promoter operably linked to the nucleic acid sequence encoding the catalytically inactive RfxCas13d; and a polyadenylation signal, wherein the resulting chimeric RNA molecule comprises the trans-spliced nucleic acid sequence; and wherein the resulting chimeric RNA molecule can restore one or more aspects of cellular homeostasis and/or cellular functionality and/or metabolic dysregulation.

Disclosed herein is a method of treating and/or preventing a genetic disease or disorder, the method comprising generating a chimeric RNA molecule in one or more cells by administering to a subject in need thereof (i) a vector comprising an isolated nucleic acid molecule comprising a nucleic acid sequence to be trans-spliced to a target endogenous pre-mRNA; a 5′ hemi intron linked to the nucleic acid sequence to be trans-spliced; one or more guide RNA sequences; a promoter operably linked to the one or more guide RNA sequences; and a nucleic acid sequence encoding an RNA binding protein, and (ii) a vector comprising an isolated nucleic acid molecule comprising a nucleic acid sequence encoding a catalytically inactive PspdCas13b, a promoter operably linked to the nucleic acid sequence encoding the catalytically inactive PspdCas13b; and a polyadenylation signal, wherein the resulting chimeric RNA molecule comprises the trans-spliced nucleic acid sequence; and wherein the resulting chimeric RNA molecule can restore one or more aspects of cellular homeostasis and/or cellular functionality and/or metabolic dysregulation.

Disclosed herein is a method of treating and/or preventing a genetic disease or disorder, the method comprising generating a chimeric RNA molecule in one or more cells by administering to a subject in need thereof (i) a vector comprising an isolated nucleic acid molecule comprising a nucleic acid sequence to be trans-spliced to a target endogenous pre-mRNA; a 3′ hemi intron; a 5′ hemi intron; two or more guide RNA sequences; a promoter operably linked to the two or more guide RNA sequences; and a nucleic acid sequence encoding one or more RNA binding proteins; and (ii) a vector comprising an isolated nucleic acid molecule comprising a nucleic acid sequence encoding a catalytically inactive RfxCas13d; a promoter operably linked to the nucleic acid sequence encoding the catalytically inactive RfxCas13d; and a polyadenylation signal, wherein the resulting chimeric RNA molecule comprises the trans-spliced nucleic acid sequence; and wherein the resulting chimeric RNA molecule can restore one or more aspects of cellular homeostasis and/or cellular functionality and/or metabolic dysregulation.

Disclosed herein is a method of treating and/or preventing a genetic disease or disorder, the method comprising generating a chimeric RNA molecule in one or more cells by administering to a subject in need thereof (i) a vector comprising an isolated nucleic acid molecule comprising a nucleic acid sequence to be trans-spliced to a target endogenous pre-mRNA; a 3′ hemi intron linked to the nucleic acid sequence to be trans-spliced; one or more guide RNA sequences; a promoter operably linked to the one or more guide RNA sequences; and a nucleic acid sequence encoding an RNA binding protein; and (ii) a vector comprising an isolated nucleic acid molecule, comprising a nucleic acid sequence encoding a catalytically inactive RfxCas13d; a promoter operably linked to the nucleic acid sequence encoding the catalytically inactive RfxCas13d; and a polyadenylation signal, wherein the resulting chimeric RNA molecule comprises the trans-spliced nucleic acid sequence; and wherein the resulting chimeric RNA molecule can restore one or more aspects of cellular homeostasis and/or cellular functionality and/or metabolic dysregulation.

### VII. DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is +10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, “isolated” refers to a nucleic acid molecule or a nucleic acid sequence that has been substantially separated, produced apart from, or purified away from other biological components in the cell or tissue of an organism in which the component occurs, such as other cells, chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins. Isolated Cas13d proteins or nucleic acids, or cells containing such, in some examples are at least 50% pure, such as at least 75%, at least 80%, at least 90%, at least 95%, at least 98%, or at least 100% pure.

As used herein, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have a disease or disorder, be suspected of having a disease or disorder, or be at risk of developing a disease or disorder (e.g., a genetic disease or disorder).

As used herein, a “regulatory element” can refer to promoters, enhancers, internal ribosomal entry sites (IRES), and other expression control elements (e.g., transcription termination signals, such as polyadenylation signals and poly-U sequences). Regulatory elements can include those that direct constitutive expression of a nucleotide sequence in many types of host cells and those that direct expression of the nucleotide sequence only in certain host cells (e.g., tissue-specific regulatory sequences).

As used herein, the term “diagnosed” means having been subjected to an examination by a person of skill, for example, a physician, and found to have a condition that can be diagnosed or treated by one or more of the disclosed isolated nucleic acid molecules, disclosed vectors, disclosed pharmaceutical formulations, or a combination thereof, or by one or more of the disclosed methods. For example, “diagnosed with a disease or disorder” means having been subjected to an examination by a person of skill, for example, a physician, and found to have a condition (such as a genetic disease or disorder) that can be treated by one or more of the disclosed isolated nucleic acid molecules, disclosed vectors, disclosed pharmaceutical formulations, or a combination thereof, or by one or more of the disclosed methods. For example, “suspected of having a disease or disorder” can mean having been subjected to an examination by a person of skill, for example, a physician, and found to have a condition (such as a genetic disease or disorder) that can likely be treated by one or more of by one or more of the disclosed isolated nucleic acid molecules, disclosed vectors, disclosed pharmaceutical formulations, or a combination thereof, or by one or more of the disclosed methods. In an aspect, an examination can be physical, can involve various tests (e.g., blood tests, genotyping, biopsies, etc.) and assays (e.g., enzymatic assay), or a combination thereof.

A “patient” refers to a subject afflicted with a disease or disorder (e.g., a genetic disease or disorder). In an aspect, a patient can refer to a subject that has been diagnosed with or is suspected of having a disease or disorder. In an aspect, a patient can refer to a subject that has been diagnosed with or is suspected of having a disease or disorder and is seeking treatment or receiving treatment for a disease or disorder.

As used herein, the phrase “identified to be in need of treatment for a disease or disorder,” or the like, refers to selection of a subject based upon need for treatment of the disease or disorder. For example, a subject can be identified as having a need for treatment of a disease or disorder (e.g., a genetic disease or disorder) based upon an earlier diagnosis by a person of skill and thereafter subjected to treatment for the genetic disease or disorder. In an aspect, the identification can be performed by a person different from the person making the diagnosis. In an aspect, the administration can be performed by one who performed the diagnosis.

As used herein, “inhibit,” “inhibiting”, and “inhibition” mean to diminish or decrease an activity, level, response, condition, severity, disease, or other biological parameter. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having a disease or disorder such as a genetic disease or disorder). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to native or control levels. In an aspect, the inhibition or reduction can be β-25%, 25-50%, 50-75%, or 75-100% as compared to native or control levels. In an aspect, a native or control level can be a pre-disease or pre-disorder level.

The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating a disease or disorder can reduce the severity of an established a disease or disorder in a subject by 1%-100% as compared to a control (such as, for example, an individual not having a genetic disease or disorder). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a disease or disorder (such as a genetic disease or disorder). For example, treating a disease or disorder can reduce one or more symptoms of a disease or disorder in a subject by 1%-100% as compared to a control (such as, for example, an individual not having a genetic disease or disorder). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established a disease or disorder. It is understood that treatment does not necessarily refer to a cure or complete ablation or eradication of a disease or disorder. However, in an aspect, treatment can refer to a cure or complete ablation or eradication of a disease or disorder.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a disease or disorder having chromatin deregulation and/or chromatin dysregulation is intended. The words “prevent”, “preventing”, and “prevention” also refer to prophylactic or preventative measures for protecting or precluding a subject (e.g., an individual) not having a given a disease or disorder (such as a genetic disease or disorder) a or related complication from progressing to that complication.

As used herein, the terms “administering” and “administration” refer to any method of providing one or more of the disclosed isolated nucleic acid molecules, disclosed vectors, disclosed pharmaceutical formulations, or a combination thereof to a subject. Such methods are well known to those skilled in the art and include, but are not limited to, the following: oral administration, transdermal administration, administration by inhalation, nasal administration, topical administration, in utero administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of a disclosed isolated nucleic acid molecule, a disclosed vector, a disclosed pharmaceutical composition, a disclosed therapeutic agent, a disclosed immune modulator, a disclosed proteasome inhibitor, a disclosed small molecule, a disclosed endonuclease, a disclosed oligonucleotide, and/or a disclosed RNA therapeutic can comprise administration directly into the CNS or the PNS. Administration can be continuous or intermittent. Administration can comprise a combination of one or more route.

In an aspect, the skilled person can determine an efficacious dose, an efficacious schedule, and an efficacious route of administration for one or more of the disclosed isolated nucleic acid molecules, disclosed vectors, disclosed pharmaceutical formulations, or a combination thereof to treat or prevent a disease or disorder (such as genetic disease or disorder). In an aspect, the skilled person can also alter, change, or modify an aspect of an administering step to improve efficacy of one or more of the disclosed isolated nucleic acid molecules, disclosed vectors, disclosed pharmaceutical formulations, or a combination thereof.

By “determining the amount” is meant both an absolute quantification of a particular analyte (e.g., an mRNA sequence containing a particular tag) or a determination of the relative abundance of a particular analyte (e.g., an amount as compared to a mRNA sequence including a different tag). The phrase includes both direct or indirect measurements of abundance (e.g., individual mRNA transcripts may be quantified or the amount of amplification of an mRNA sequence under certain conditions for a certain period may be used a surrogate for individual transcript quantification) or both.

As used herein, “modifying the method” can comprise modifying or changing one or more features or aspects of one or more steps of a disclosed method. For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, by changing the duration of time one or more of the disclosed isolated nucleic acid molecules, disclosed vectors, disclosed pharmaceutical formulations, or a combination are administered to a subject, or by substituting for one or more of the disclosed components and/or reagents with a similar or equivalent component and/or reagent. The same applies to all disclosed therapeutic agents, immune modulators, immunosuppressive agents, proteosome inhibitors, etc.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples ofliquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

As used herein, “concurrently” means (1) simultaneously in time, or (2) at different times during the course of a common treatment schedule.

The term “contacting” as used herein refers to bringing one or more of the disclosed isolated nucleic acid molecules, disclosed vectors, disclosed pharmaceutical formulations, or a combination thereof together with a target area or intended target area in such a manner that the one or more of the disclosed isolated nucleic acid molecules, disclosed vectors, disclosed pharmaceutical formulations, or a combination thereof exert an effect on the intended target or targeted area either directly or indirectly. A target area can comprise one or more cells, and in an aspect, one or more cells can be in a subject. A target area or intended target area can be one or more of a subject's organs (e.g., lungs, heart, liver, kidney, brain, etc.). In an aspect, a target area or intended target area can be any cell or any organ infected by a disease or disorder (such as a genetic disease or disorder). In an aspect, a target area or intended target area can be any organ, tissue, or cells that are affected by a disease or disorder (such as a genetic disease or disorder).

As used herein, “determining” can refer to measuring or ascertaining the presence and severity of a disease or disorder, such as, for example, a genetic disease or disorder. Methods and techniques used to determine the presence and/or severity of a disease or disorder are typically known to the medical arts. For example, the art is familiar with the ways to identify and/or diagnose the presence, severity, or both of a disease or disorder (such as, for example, a genetic disease or disorder).

As used herein, “effective amount” and “amount effective” can refer to an amount that is sufficient to achieve the desired result such as, for example, the treatment and/or prevention of a disease or disorder (e.g., a genetic disease or disorder) or a suspected disease or disorder. As used herein, the terms “effective amount” and “amount effective” can refer to an amount that is sufficient to achieve the desired an effect on an undesired condition (e.g., a disease or disorder). For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of a disclosed isolated nucleic acid molecule, a disclosed vector, or a disclosed pharmaceutical formulation; that (i) treats the particular disease, condition, or disorder (e.g., a genetic disease or disorder), (ii) attenuates, ameliorates, or eliminates one or more symptoms of the particular disease, condition, or disorder e.g., a genetic disease or disorder), or (iii) delays the onset of one or more symptoms of the particular disease, condition, or disorder described herein (e.g., a genetic disease or disorder). The specific therapeutically effective dose level for any particular patient will depend upon a variety of factors including the disorder being treated and the severity of the disorder; the disclosed isolated nucleic acid molecules, disclosed vectors, disclosed pharmaceutical formulations employed; the disclosed methods employed; the age, body weight, general health, sex and diet of the patient; the time of administration; the route of administration; the rate of excretion of the disclosed isolated nucleic acid molecules, disclosed vectors, or disclosed pharmaceutical formulations employed; the duration of the treatment; drugs used in combination or coincidental with the disclosed isolated nucleic acid molecules, disclosed vectors, or disclosed pharmaceutical formulations employed, and other like factors well known in the medical arts. For example, it is well within the skill of the art to start doses of the disclosed isolated nucleic acid molecules, disclosed vectors, or disclosed pharmaceutical formulations at levels lower than those required to achieve the desired therapeutic effect and to gradually increase the dosage until the desired effect is achieved. If desired, then the effective daily dose can be divided into multiple doses for purposes of administration. Consequently, a single dose of the disclosed isolated nucleic acid molecules, disclosed vectors, or disclosed pharmaceutical formulations can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, a disease or disorder due to a missing, deficient, and/or mutant protein or enzyme.

As used herein, “RNA therapeutics” can refer to the use of oligonucleotides to target RNA. RNA therapeutics can offer the promise of uniquely targeting the precise nucleic acids involved in a particular disease with greater specificity, improved potency, and decreased toxicity. This could be particularly powerful for genetic diseases where it is most advantageous to aim for the RNA as opposed to the protein. In an aspect, a therapeutic RNA can comprise one or more expression sequences. As known to the art, expression sequences can comprise an RNAi, shRNA, mRNA, non-coding RNA (ncRNA), an antisense such as an antisense RNA, miRNA, morpholino oligonucleotide, peptide-nucleic acid (PNA) or ssDNA (with natural, and modified nucleotides, including but not limited to, LNA, BNA, 2′-O-Me-RNA, 2′-MEO-RNA, 2′-F-RNA), or analog or conjugate thereof. In an aspect, a disclosed therapeutic RNA can comprise one or more long non-coding RNA (lncRNA), such as, for example, a long intergenic non-coding RNA (lincRNA), pre-transcript, pre-miRNA, pre-mRNA, competing endogenous RNA (ceRNA), small nuclear RNA (snRNA), small nucleolar RNA (snoRNA), pseudo-gene, rRNA, or tRNA. In an aspect, ncRNA can be piwi-interacting RNA (piRNA), primary miRNA (pri-miRNA), or premature miRNA (pre-miRNA). In an aspect, a disclosed therapeutic RNA or an RNA therapeutic can comprise antisense oligonucleotides (ASOs) that inhibit mRNA translation, oligonucleotides that function via RNA interference (RNAi) pathway, RNA molecules that behave like enzymes (ribozymes), RNA oligonucleotides that bind to proteins and other cellular molecules, and ASOs that bind to mRNA and form a structure that is recognized by RNase H resulting in cleavage of the mRNA target. In an aspect, RNA therapeutics can comprise RNAi and ASOs that inhibit mRNA translation. Generally speaking, as known to the art, RNAi operates sequence specifically and post-transcriptionally by activating ribonucleases which, along with other enzymes and complexes, coordinately degrade the RNA after the original RNA target has been cut into smaller pieces while antisense oligonucleotides bind to their target nucleic acid via Watson-Crick base pairing, and inhibit or alter gene expression via steric hindrance, splicing alterations, initiation of target degradation, or other events.

As used herein, “small molecule” can refer to any organic or inorganic material that is not a polymer. Small molecules exclude large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weight of over 2,000, 3,000, 4,000, 5,000, 6,000, 7,000, 8,000, 9,000, or 10,000). In an aspect, a “small molecule”, for example, can be a drug that can enter cells easily because it has a low molecular weight. In an aspect, a small molecule can be used in conjunction with a disclosed composition in a disclosed method.

As used herein, “operably linked” means that expression of a gene or a transgene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

As used herein, “peptide,” “polypeptide,” and “protein” are used interchangeably, and refer to a compound comprised of amino acid residues covalently linked by peptide bonds. A protein must contain at least two amino acids and there is no limitation on the maximum number of amino acids that can comprise a protein's sequence. The term “peptide” can refer to a short chain of amino acids including, for example, natural peptides, recombinant peptides, synthetic peptides, or any combination thereof. Proteins and peptides can include, for example, biologically active fragments, substantially homologous polypeptides, oligopeptides, homodimers, heterodimers, variants of polypeptides, modified polypeptides, derivatives, analogs, and fusion proteins, among others.

“Nucleic acid” or “oligonucleotide” or “polynucleotide” as used herein means at least two nucleotides covalently linked together. The depiction of a single strand can also define the sequence of the complementary strand. Thus, a nucleic acid can encompass the complementary strand of a depicted single strand. Many variants of a nucleic acid can be used for the same purpose as a given nucleic acid. Thus, a nucleic acid can encompass substantially identical nucleic acids and complements thereof. A single strand can provide a probe that can hybridize to a target sequence under stringent hybridization conditions. Thus, a nucleic acid can encompass a probe that hybridizes under stringent hybridization conditions. A nucleic acid can be single-stranded, or double-stranded, or can contain portions of both double-stranded and single-stranded sequence.

The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods. Also as used herein, the terms “nucleic acid,” “nucleic acid molecule,” “nucleic acid construct,” “nucleotide sequence”, and “polynucleotide” can refer to RNA or DNA that is linear or branched, single or double stranded, or a hybrid thereof. The term can encompass RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. A “synthetic” nucleic acid or polynucleotide, as used herein, refers to a nucleic acid or polynucleotide that is not found in nature but is constructed by the hand of man and therefore is not a product of nature.

A “polynucleotide” is a sequence of nucleotide bases, and may be RNA, DNA, or DNA-RNA hybrid sequences (including both naturally occurring and non-naturally occurring nucleotides).

A “fragment” or “portion” of a nucleotide sequence can be understood to mean a nucleotide sequence of reduced length relative (e.g., reduced by 1, 2, 3, 4, 5, 6, 7, 8, 9, 10, 11, 12, 13, 14, 15, 16, 17, 18, 19, 20 or more nucleotides) to a reference nucleic acid or nucleotide sequence and comprising, consisting essentially of, or consisting of a nucleotide sequence of contiguous nucleotides identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleic acid or nucleotide sequence. Such a nucleic acid fragment or portion according to the disclosure can be, where appropriate, included in a larger polynucleotide of which it is a constituent. In an aspect, a fragment or portion of a nucleotide sequence or nucleic acid sequence can comprise the sequence encoding an exon having one or more mutations.

A “fragment” or “portion” of an amino acid sequence can be understood to mean an amino acid sequence of reduced length relative (e.g., reduced by 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 250, or more amino acids) to a reference amino acid sequence and comprising, consisting essentially of, or consisting of an amino acid sequence of contiguous amino acids identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference amino acid sequence. Such an amino acid fragment or portion according to the disclosure can be, where appropriate, included in a larger amino acid sequence of which it is a constituent.

A “heterologous” or a “recombinant” nucleotide or amino acid sequence as used interchangeably herein can refer to a nucleotide or an amino acid sequence not naturally associated with a host cell into which it is introduced, including non-naturally occurring multiple copies of a naturally occurring nucleotide or amino acid sequence.

Different nucleic acids or proteins having homology can be referred to as “homologues”. The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the disclosed compositions and disclosed methods can comprise homologues to the disclosed nucleotide sequences and/or disclosed polypeptide sequences.

“Orthologous,” as used herein, can refer to homologous nucleotide sequences and/or amino acid sequences in different species that arose from a common ancestral gene during speciation. A homologue of a disclosed nucleotide sequence or a disclosed polypeptide can have substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100%) to a disclosed nucleotide sequence or a disclosed polypeptide.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

As used herein, “promoter” or “promoters” are known to the art. Depending on the level and tissue-specific expression desired, a variety of promoter elements can be used. A promoter can be tissue-specific or ubiquitous and can be constitutive or inducible, depending on the pattern of the gene expression desired. A promoter can be native (endogenous) or foreign (exogenous) and can be a natural or a synthetic sequence. By foreign or exogenous, it is intended that the transcriptional initiation region is not found in the wild-type host into which the transcriptional initiation region is introduced.

“Tissue-specific promoters” are known to the art and include, but are not limited to, neuron-specific promoters, muscle-specific promoters, liver-specific promoters, skeletal muscle-specific promoters, and heart-specific promoters.

“Liver-specific promoters” are known to the art and include, but are not limited to, the thyroxin binding globulin (TBG) promoter, the al-microglobulin/bikunin enhancer/thyroid hormone-binding globulin promoter, the human albumin (hALB) promoter, the thyroid hormone-binding globulin promoter, the α-1-anti-trypsin promoter, the bovine albumin (bAlb) promoter, the murine albumin (mAlb) promoter, the human al-antitrypsin (hAAT) promoter, the ApoEhAAT promoter comprising the ApoE enhancer and the hAAT promoter, the transthyretin (TTR) promoter, the liver fatty acid binding protein promoter, the hepatitis B virus (HBV) promoter, the DC172 promoter comprising the hAAT promoter and the al-microglobulin enhancer, the DC190 promoter comprising the human albumin promoter and the prothrombin enhancer, or any other natural or synthetic liver-specific promoter. In an aspect, a liver specific promoter can comprise about 845-bp and comprise the thyroid hormone-binding globulin promoter sequences (2382 to 13), two copies of al-microglobulin/bikunin enhancer sequences (22,804 through 22,704), and a 71-bp leader sequence as described by Ill CR, et al. (1997). In an aspect, a disclosed liver specific promoter can comprise the sequence set forth in SEQ ID NO:32, or a sequence having about 50%, about 60%, about 70% about 80%, about 90%, about 95%, or more identity to the sequence set forth in SEQ ID NO:32.

Ubiquitous/constitutive promoters” are known to the art and include, but are not limited to, a CMV major immediate-early enhancer/chicken beta-actin promoter, a cytomegalovirus (CMV) major immediate-early promoter, an Elongation Factor 1-α (EF1-α) promoter, a simian vacuolating virus 40 (SV40) promoter, an AmpR promoter, a PγK promoter, a human ubiquitin C gene (Ubc) promoter, a MFG promoter, a human beta actin promoter, a CAG promoter, a EGR1 promoter, a FerH promoter, a FerL promoter, a GRP78 promoter, a GRP94 promoter, a HSP70 promoter, a β-kin promoter, a murine phosphoglycerate kinase (mPGK) or human PGK (hPGK) promoter, a ROSA promoter, human Ubiquitin B promoter, a Rous sarcoma virus promoter, or any other natural or synthetic ubiquitous/constitutive promoters.

As used herein, an “inducible promoter” refers to a promoter that can be regulated by positive or negative control. Factors that can regulate an inducible promoter include, but are not limited to, chemical agents (e.g., the metallothionein promoter or a hormone inducible promoter), temperature, and light.

As used herein, the term “serotype” is a distinction used to refer to an AAV having a capsid that is serologically distinct from other AAV serotypes. Serologic distinctiveness can be determined by the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes).

As used herein, “tropism” refers to the specificity of an AAV capsid protein present in an AAV viral particle, for infecting a particular type of cell or tissue. The tropism of an AAV capsid for a particular type of cell or tissue may be determined by measuring the ability of AAV vector particles comprising the hybrid AAV capsid protein to infect or to transduce a particular type of cell or tissue, using standard assays that are well-known in the art such as those disclosed in the examples of the present application. As used herein, the term “liver tropism” or “hepatic tropism” refers to the tropism for liver or hepatic tissue and cells, including hepatocytes.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of a missing, deficient, and/or mutant protein or enzyme. It should be understood that sequence with substantial sequence identity do not necessarily have the same length and may differ in length. For example, sequences that have the same nucleotide sequence but of which one has additional nucleotides on the 3′- and/or 5′-side are 100% identical.

As used herein, “codon optimization” can refer to a process of modifying a nucleic acid sequence for enhanced expression in the host cells of interest by replacing one or more codons or more of the native sequence with codons that are more frequently or most frequently used in the genes of that host cell while maintaining the native amino acid sequence. Various species exhibit particular bias for certain codons of a particular amino acid. As contemplated herein, genes can be tailored for optimal gene expression in a given organism based on codon optimization. Codon usage tables are readily available, for example, at the “Codon Usage Database.” Many methods and software tools for codon optimization have been reported previously. (See, for example, genomes.urv.es/OPTIMIZER/).

As used herein, “RNA Editing” can refer to a type of genetic engineering in which an RNA molecule (or ribonucleotides of the RNA) is inserted, deleted, or replaced in the genome of an organism using engineered nucleases (such as the Cas13d proteins provided herein), which create site-specific strand breaks at desired locations in the RNA. The induced breaks are repaired resulting in targeted mutations or repairs.

As used herein, “CRISPR or clustered regularly interspaced short palindromic repeat” is an ideal tool for correction of genetic abnormalities as the system can be designed to target genomic DNA directly. Cas9 is well-known to the art. The CRISPR/Cas methods disclosed herein, such as those that use an Cas13d, can be used to edit the sequence of one or more target RNAs, such as one associated with a disease or disorder disclosed herein (e.g., a genetic disease or disorder).

The diverse Cas13 family contains at least four known subtypes, including Cas13a (formerly C2c2), Cas13b, Cas13c, and Cas13d. All known Cas13 family members contain two HEPN domains, which confer RNase activity. Cas13 can be reprogrammed to cleave a targeted ssRNA molecule through a short guide RNA with complementarity to the target sequence. Cas13s function similarly to Cas9, using a ˜64-nucleotide guide RNA to encode target specificity. The Cas13 protein complexes with the guide RNA via recognition of a short hairpin in the crRNA, and target specificity is encoded by a 28-nucleotide to a 30-nucleotide spacer that is complementary to the target region. In addition to programmable RNase activity, all Cas13s exhibit collateral activity after recognition and cleavage of a target transcript, leading to non-specific degradation of any nearby transcripts regardless of complementarity to the spacer. While Cas13a showed some activity for RNA knockdown, certain orthologs of Cas13b proved more stable and robust in mammalian cells for RNA knockdown and editing. More recently, additional orthologs of Cas13 have been discovered, including Cas13d, which has been leveraged for efficient and robust knockdown across many endogenous transcripts. Cas13d can be used to modulate splicing of endogenous transcripts and that the coding sequence for Cas13d is small enough to fit within the packaging limits of AAV for in vivo delivery.

In an aspect, Cas13 can be considered an outlier in the CRISPR world because it targets RNA, not DNA. Once it is activated by a ssRNA sequence bearing complementarity to its crRNA spacer, it unleashes a nonspecific RNase activity and destroys all nearby RNA regardless of their sequence. As disclosed herein, this property can be harnessed in vitro for precision diagnostics. Generally, Cas13 can be found in Leptotrichia buccalis, Leptotrichia shahii, Ruminococcus flavefaciens, Bergeyella zoohelcum, Prevotella buccae, and Listeria seeligeri and can have a size of about 900 to about 1300 amino acids. In an aspect, the guide spacer length can be about 22 to about 30 nucleotides while the total guide length can be about 52 to about 66 nucleotides. In an aspect, a PAM can be 3-H for LshCas13a, 5-D and 3-NAN or NNA for BzCas13b, and none for RfCas13d. In an aspect, a disclosed Cas13 can cut ssRNA.

As known to the skilled person in the cart, a Cas13d ortholog can be from a prokaryotic genome or metagenome, gut metagenome, an activated sludge metagenome, an anaerobic digester metagenome, a chicken gut metagenome, a human gut metagenome, a pig gut metagenome, a bovine gut metagenome, a sheep gut metagenome, a goat gut metagenome, a capybara gut metagenome, a primate gut metagenome, a termite gut metagenome, a fecal metagenome, a genome from the Order Clostridiales, or the Family Ruminococcaceae. In an aspect, a disclosed Cas13d ortholog can include an Cas13d ortholog from Ruminococcus albus, Eubacterium siraeum, a Ruminococcus flavefaciens strain XPD3002, Ruminococcus flavefaciens FD-1, uncultured Eubacterium sp TS28-c4095, uncultured Ruminococcus sp., Ruminococcus bicirculans, or Ruminococcus sp CAG57.

In an aspect, a disclosed Cas13 can comprise RfxCas13d (see, for example, US Patent Publication No. 2020/0244609, which is incorporated by reference for its teachings of RfxCas13d and relevant sequences). In an aspect, a disclosed Cas13 can comprise PspCas13b (see, for example, US Patent Publication No. 2020/0231975, which is incorporated by reference for its teachings of PspCas13b and relevant sequences).

As known to the art, RNA binding proteins consist of multiple repetitive sequences that contain only a few specific basic domains. Structurally, common RNA-binding domains mainly include RNA-recognition motif (RRM), K homology (KH) domain, double-stranded RBD (dsRBD), cold-shock domain (CSD), arginine-glycine-glycine (RGG) motif, tyrosine-rich domain, and zinc fingers (ZnF) of the CCHC, CCCH, ZZ type etc. According to the different functions of RBPs in cells, RBPs can be divided into epithelial splicing regulatory proteins (ESRP1), cytoplasmic polyadenylation element binding protein family (CPEB1/2), Hu-antigen R (HuR), heterogeneous nuclear ribonucleoprotein family members (hnRNP A/D/H/K/M/E/L), insulin-like growth factor 2 mRNA family members (IMP1/2/3), zfh family of transcription factors (ZEB1/2), KH-type splicing regulatory protein (KHSRP), La ribonucleoprotein domain family members (LARP1/6/7), Lin-28 homolog proteins (Lin28), Musashi protein family (MSI1/2), Pumilio protein family (PUM1/2), Quaking (QK), RNA-binding motif protein family (4/10/38/47), Src-associated substrate during mitosis of 68 kDa (SAM68), serine and arginine rich splicing factor (SRSF1/3), T cell intracellular antigens (TIA1/TIAR), and Upstream of N-Ras (UNR).

As used herein, “immune tolerance,” “immunological tolerance,” and “immunotolerance” refers to a state of unresponsiveness or blunted response of the immune system to substances (e.g., a disclosed isolated nucleic acid molecule, a disclosed vector, a disclosed transgene product, a disclosed pharmaceutical formulation, a disclosed therapeutic agent, etc.) that have the capacity to elicit an immune response in a subject. Immune tolerance is induced by prior exposure to a specific antigen. Immune tolerance can be determined in a subject by measuring antibodies against a particular antigen or by liver-restricted transgene expression with a viral vector (such as, for example, AAV). Low or absent antibody titers over time is an indicator of immune tolerance. For example, in some embodiments, immune tolerance can be established by having IgG antibody titers of less than or equal to about 12,000, 11,500, 11,000, 10,500, 10,000, 9,500, 9,000, 8,500, 8,000, 7,500, 7,000, 6,500, or 6,000 within following gene therapy (such as the administration of the transgene encoding, for example, a missing, deficient, and/or mutant protein or enzyme).

As known to the art, antibodies (Abs) can mitigate AAV infection through multiple mechanisms by binding to AAV capsids and blocking critical steps in transduction such as cell surface attachment and uptake, endosomal escape, productive trafficking to the nucleus, or uncoating as well as promoting AAV opsonization by phagocytic cells, thereby mediating their rapid clearance from the circulation. For example, in humans, serological studies reveal a high prevalence of NAbs in the worldwide population, with about 67% of people having antibodies against AAV1, 72% against AAV2, and approximately 40% against AAV serotypes 5 through 9. Vector immunogenicity represents a major challenge in re-administration of AAV vectors.

In an aspect, also disclosed herein are partial self-complementary parvovirus (e.g., a disclosed AAV) genomes, plasmid vectors encoding the parvovirus genomes, and parvovirus (e.g., a disclosed AAV) particles including such genomes. In an aspect, provided herein is a plasmid vector comprising a nucleotide sequence encoding a disclosed parvovirus genome such as for example, a disclosed AAV. In an aspect, provided herein is a partial self-complementary parvovirus genome including a payload construct, parvovirus ITRs flanking the payload construct, and a self-complementary region flanking one of the ITRs. A self-complementary region can comprise a nucleotide sequence that is complementary to the payload construct. A disclosed self-complementary region can have a length that is less the entire length of the payload construct.

In an aspect, a disclosed self-complementary region of a disclosed parvovirus genome can comprise a minimum length, while still having a length that is less the entire length of the payload construct. In an aspect, a disclosed self-complementary region can comprise at least 50 bases in length, at least 100 bases in length, at least 200 in length, at least 300 bases in length, at least 400 bases in length, at least 500 bases in length, at least 600 bases in length, at least 700 bases in length, at least 800 bases in length, at least 900 bases in length, or at least 1,000 bases in length.

In an aspect, a “self-complementary parvovirus genome” can be a single stranded polynucleotide having, in the 5′ to 3′ direction, a first parvovirus ITR sequence, a heterologous sequence (e.g., payload construct comprising, for example, a desired gene), a second parvovirus ITR sequence, a second heterologous sequence, wherein the second heterologous sequence is complementary to the first heterologous sequence, and a third parvovirus ITR sequence. In contrast to a self-complementary genome, a “partial self-complementary genome” does not include three parvovirus ITRs and the second heterologous sequence that is complementary to the first heterologous sequence has a length that is less than the entire length of the first heterologous sequence (e.g., payload construct). Accordingly, a partial self-complementary genome is a single stranded polynucleotide having, in the 5′ to 3′ direction or the 3′ to 5′ direction, a first parvovirus ITR sequence, a heterologous sequence (e.g., payload construct), a second parvovirus ITR sequence, and a self-complementary region that is complementary to a portion of the heterologous sequence and has a length that is less than the entire length the heterologous sequence.

As used herein, “immune-modulating” refers to the ability of a disclosed isolated nucleic acid molecules, a disclosed vector, a disclosed pharmaceutical formulation, or a disclosed agent to alter (modulate) one or more aspects of the immune system. The immune system functions to protect the organism from infection and from foreign antigens by cellular and humoral mechanisms involving lymphocytes, macrophages, and other antigen-presenting cells that regulate each other by means of multiple cell-cell interactions and by elaborating soluble factors, including lymphokines and antibodies, that have autocrine, paracrine, and endocrine effects on immune cells.

As used herein, “immune modulator” refers to an agent that is capable of adjusting a given immune response to a desired level (e.g., as in immunopotentiation, immunosuppression, or induction of immunologic tolerance). Examples of immune modulators include but are not limited to, a disclosed immune modulator can comprise aspirin, azathioprine, belimumab, betamethasone dipropionate, betamethasone valerate, bortezomib, bredinin, cyazathioprine, cyclophosphamide, cyclosporine, deoxyspergualin, didemnin B, fluocinolone acetonide, folinic acid, ibuprofen, IL6 inhibitors (such as sarilumab) indomethacin, inebilizumab, intravenous gamma globulin (IVIG), methotrexate, methylprednisolone, mycophenolate mofetil, naproxen, prednisolone, prednisone, prednisolone indomethacin, rapamycin, rituximab, sirolimus, sulindac, synthetic vaccine particles containing rapamycin (SVP-Rapamycin or ImmTOR), thalidomide, tocilizumab, tolmetin, triamcinolone acetonide, anti-CD3 antibodies, anti-CD4 antibodies, anti-CD19 antibodies, anti-CD20 antibodies, anti-CD22 antibodies, anti-CD40 antibodies, anti-FcRN antibodies, anti-IL6 antibodies, anti-IGF1R antibodies, an IL2 mutein, a BTK inhibitor, or a combination thereof. In an aspect, a disclosed immune modulator can comprise one or more Treg (regulatory T cells) infusions (e.g., antigen specific Treg cells to AAV). In an aspect, a disclosed immune modulator can be bortezomib or SVP-Rapamycin. In an aspect, an immune modulator can be administered by any suitable route of administration including, but not limited to, in utero, intra-CSF, intrathecally, intravenously, subcutaneously, transdermally, intradermally, intramuscularly, orally, transcutaneously, intraperitoneally (IP), or intravaginally. In an aspect, a disclosed immune modulator can be administered using a combination of routes. Administration can also include hepatic intra-arterial administration or administration through the hepatic portal vein (HPV). Administration of an immune modulator can be continuous or intermittent, and administration can comprise a combination of one or more routes.

As used herein, the term “immunotolerant” refers to unresponsiveness to an antigen (e.g., a vector, a therapeutic protein, a transgene product, etc.). An immunotolerant promoter can reduce, ameliorate, or prevent transgene-induced immune responses that can be associated with gene therapy. Assays known in the art to measure immune responses, such as immunohistochemical detection of cytotoxic T cell responses, can be used to determine whether one or more promoters can confer immunotolerant properties.

As used herein, the term “package insert” is used to refer to instructions customarily included in commercial packages of therapeutic products, that contain information about the indications, usage, dosage, administration, contraindications and/or warnings concerning the use of such therapeutic products.

As used herein, the term “in combination” in the context of the administration of other therapies (e.g., other agents) includes the use of more than one therapy (e.g., drug therapy).

Administration “in combination with” one or more further therapeutic agents includes simultaneous (e.g., concurrent) and consecutive administration in any order. The use of the term “in combination” does not restrict the order in which therapies are administered to a subject. By way of non-limiting example, a first therapy (e.g., a disclosed isolated nucleic acid molecule, a disclosed vector, a disclosed pharmaceutical formulation, or a combination thereof) may be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., agent) to a subject having or diagnosed with a disease or disorder (such as a genetic disease or disorder).

Disclosed are the components to be used to prepare the disclosed isolated nucleic acid molecules, disclosed vectors, or disclosed pharmaceutical formulations as well as the disclosed isolated nucleic acid molecules, disclosed vectors, or disclosed pharmaceutical formulations used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### B. Compositions for Transcriptome Engineering

1. Nucleic Acid Molecules
a. 5′ Replacement Constructs

Disclosed herein is an isolated nucleic acid molecule comprising a nucleic acid sequence to be trans-spliced to a target endogenous pre-mRNA; a 5′ hemi intron linked to the nucleic acid sequence to be trans-spliced; one or more guide RNA sequences; a promoter operably linked to the one or more guide RNA sequences; and a nucleic acid sequence encoding an RNA binding protein.

In an aspect, a disclosed isolated nucleic acid molecule can restore one or more aspects of cellular homeostasis and/or cellular functionality and/or metabolic dysregulation. In an aspect, a disclosed isolated nucleic acid molecule can restore the functionality and/or structural integrity of a missing, deficient, and/or mutant protein or enzyme.

In an aspect, a disclosed isolated nucleic acid molecule can further comprise a polyadenylation signal.

In an aspect, a disclosed isolated nucleic acid molecule can further comprise a spacer region. In an aspect, a disclosed spacer region can separate the 5′ splice region from the one or more guide RNA sequences. In an aspect, a disclosed spacer region can comprise any known spacer. In an aspect, a disclosed spacer region can comprise a consensus splicing motif (e.g., such as U1 or U2). In an aspect, a disclosed spacer region can comprise a limited number of consensus splicing motifs (e.g., such as U1 or U2).

In an aspect, a disclosed isolated nucleic acid molecule can further comprise one or more stem loops. In an aspect, a disclosed stem loop can be a cognate aptamer for a disclosed RNA binding protein. For example, in an aspect, a disclosed stem loop can be a direct repeat of the guide RNA scaffold for a disclosed Cas13d. In an aspect, a disclosed stem loop can facilitate interaction between a disclosed RNA molecule and a disclosed Cas protein.

In an aspect, a disclosed isolated nucleic acid molecule can further comprise a nuclear localization signal (NLS). In an aspect, a disclosed NLS can be comprise the sequence set froth in SEQ ID NO:60. In an aspect, a disclosed NLS can comprise any NLS known to the art. As known to the art (see, e.g., Lu J, et al. (2021) Cell Commun Signal. 19:60, which is incorporated herein by reference for its teachings of NLS), nuclear localization signals (NLS) are generally short peptides that act as a signal fragment that mediates the transport of proteins from the cytoplasm into the nucleus.

In an aspect, the one or more disclosed guide RNA sequences can be directed the intron immediately 5′ to the first exon of the target endogenous pre-mRNA.

In an aspect of a disclosed isolated nucleic acid molecule, a disclosed 5′ hemi intron can comprise a consensus 5′ splice site. In an aspect, a disclosed 5′ splice site can comprise the sequence set forth in SEQ ID NO:59. In an aspect, a disclosed consensus 5′ splice site can comprise the sequence set forth in SEQ ID NO:61. In an aspect, a disclosed consequence 5′ splice site can comprise MAG|GURAGU (SEQ ID NO:61), wherein | denotes the exon intron junction, wherein M=A or C, and wherein R=A or G.

In an aspect, a disclosed 5′ hemi intron can be recognized by nuclear splicing components within a host cell. In an aspect, a disclosed nucleic acid sequence encoding the RNA binding protein can interact with the one or more stem loops and/or can stabilize the one or more guide RNA sequences.

As known to the skilled person, RNA binding proteins (RBPs) can be important effectors of gene expression. RBPs can recognize hundreds of transcripts and form extensive regulatory networks that help to maintain cell homeostasis. Accordingly, the malfunction of RBPs underlies the origin of many diseases. In an aspect, a disclosed RNA binding protein can be any RNA binding protein having bispecific affinity for the trans-splicing RNA and the target pre-mRNA of interest. In an aspect, this affinity can be mediated by riboncleoprotein interactions by, for example, Type VI CRISPR enzymes, or through direct RNA protein interactions by, for example, Pumillo and FBF (PUF) proteins. In an aspect, these interactions can be mediated by protein/aptamer interactions. RNA binding proteins are discussed in depth supra.

In an aspect, a disclosed RNA binding protein can comprise bispecific affinity for a disclosed target pre-mRNA as well as a disclosed Cas13 or a disclosed catalytically inactive Cas13. In an aspect, a disclosed Cas13 can comprise any catalytically inactive Cas13. For example, in an aspect, a disclosed Cas13 can comprise a catalytically inactive RfxCas13d or a catalytically inactive PspdCas13b. For example, in an aspect, a Cas13 alternative can comprise alternatives known to the art including, but not limited to CRISPR-Inspired RNA Targeting System (CIRTS), Pumillo and FBF (PUF), rCas9, and/or any bifunctional RNA binding protein that can both bind to the trans-splicing RNA molecule and the target RNA to facilitate interaction between the two RNA species. RNA binding proteins are discussed in depth supra.

In an aspect, a disclosed nucleic acid sequence to be trans-spliced can encode a translatable protein or a portion thereof. In an aspect, a disclosed portion can comprise one or more exons comprising a mutation. In an aspect, a disclosed portion can comprise some part of the gene sequence but not the complete sequence. For example, in an aspect, a disclosed portion can comprise the nucleic acid sequence having one or more mutations.

In an aspect, a disclosed nucleic acid sequence to be trans-spliced can encode LMNA/C or a portion thereof. In an aspect, the one or more disclosed guide RNA sequences can comprise the sequence set forth in SEQ ID NO:31, SEQ ID NO:32, or SEQ ID NO:32 or a fragment thereof.

LMNA/C is known to the art (e.g., Gene ID 4000) and this nucleotide sequence can comprise nucleotides 4974-62517 in Accession No. NG008692.2. The nuclear lamina consists of a two-dimensional matrix of proteins located next to the inner nuclear membrane. The lamin family of proteins make up the matrix and are highly conserved in evolution. During mitosis, the lamina matrix is reversibly disassembled as the lamin proteins are phosphorylated. Lamin proteins are involved in nuclear stability, chromatin structure and gene expression. Vertebrate lamins consist of two types, A and B. Alternative splicing results in multiple transcript variants.

Mutations in this gene lead to several diseases: Emery-Dreifuss muscular dystrophy, familial partial lipodystrophy, limb girdle muscular dystrophy, dilated cardiomyopathy, Charcot-Marie-Tooth disease, and Hutchinson-Gilford progeria syndrome.

In an aspect, a disclosed encoded Lamin A/C (LMNA/C) can comprise the following sequence or a fragment thereof.

(SEQ ID NO: 55)
METPSQRRATRSGAQASSTPLSPTRITRLQEKEDLQELNDRLAVYIDRV
RSLETENAGLRLRITESEEVVSREVSGIKAAYEAELGDARKTLDSVAKE
RARLQLELSKVREEFKELKARNTKKEGDLIAAQARLKDLEALLNSKEAA
LSTALSEKRTLEGELHDLRGQVAKLEAALGEAKKQLQDEMLRRVDAENR
LQTMKEELDFQKNIYSEELRETKRRHETRLVEIDNGKQREFESRLADAL
QELRAQHEDQVEQYKKELEKTYSAKLDNARQSAERNSNLVGAAHEELQQ
SRIRIDSLSAQLSQLQKQLAAKEAKLRDLEDSLARERDTSRRLLAEKER
EMAEMRARMQQQLDEYQELLDIKLALDMEIHAYRKLLEGEEERLRLSPS
PTSQRSRGRASSHSSQTQGGGSVTKKRKLESTESRSSFSQHARTSGRVA
VEEVDEEGKFVRLRNKSNEDQSMGNWQIKRQNGDDPLLTYRFPPKFTLK
AGQVVTIWAAGAGATHSPPTDLVWKAQNTWGCGNSLRTALINSTGEEVA
MRKLVRSVTVVEDDEDEDGDDLLHHHHGSHCSSSGDPAEYNLRSRTVLC
GTCGQPADKASASGSGAQVGGPISSGSSASSVTVTRSYRSVGGSGGGSF
GDNLVTRSYLLGNSSPRTQSPQNCSIM.

In an aspect, a disclosed encoded Lamin A/C can comprise a sequence having at least 40%, 50%, 60%, 70%, 80%, 85%, 86%, 87%, 88%, 89%, 90%, 91%, 92%, 93%, 94%, 95%, 96%, 97%, 98%, 99% identity to the sequence set forth in SEQ ID NO:55.

In an aspect, a disclosed nucleic acid sequence to be trans-spliced can encode DP71 or a portion thereof. DP71 is known to the art (e.g., Gene ID 13405).

In an aspect, a disclosed nucleic acid sequence to be trans-spliced can encode CFTR or a portion thereof. CFTR is known to the art (e.g., Gene ID 1080) and this nucleotide sequence can comprise nucleotides 19180-207882 in Accession No. NG016465.4. This gene encodes a member of the ATP-binding cassette (ABC) transporter superfamily. The encoded protein functions as a chloride channel, making it unique among members of this protein family, and controls ion and water secretion and absorption in epithelial tissues. Channel activation is mediated by cycles of regulatory domain phosphorylation, ATP-binding by the nucleotide-binding domains, and ATP hydrolysis. Mutations in this gene cause cystic fibrosis, the most common lethal genetic disorder in populations of Northern European descent. The most frequently occurring mutation in cystic fibrosis, DeltaF508, results in impaired folding and trafficking of the encoded protein. Multiple pseudogenes have been identified in the human genome.

In an aspect, a disclosed encoded CFTR can comprise the following sequence or a fragment thereof.

(SEQ ID NO: 54)
MQRSPLEKASVVSKLFFSWTRPILRKGYRQRLELSDIYQIPSVDSADNLSEKLEREWDRE
LASKKNPKLINALRRCFFWRFMFYGIFLYLGEVTKAVQPLLLGRIIASYDPDNKEERSIAI
YLGIGLCLLFIVRTLLLHPAIFGLHHIGMQMRIAMFSLIYKKTLKLSSRVLDKISIGQLVSL
LSNNLNKFDEGLALAHFVWIAPLQVALLMGLIWELLQASAFCGLGFLIVLALFQAGLGR
MMMKYRDQRAGKISERLVITSEMIENIQSVKAYCWEEAMEKMIENLRQTELKLTRKAA
YVRYFNSSAFFFSGFFVVFLSVLPYALIKGIILRKIFTTISFCIVLRMAVTRQFPWAVQTW
YDSLGAINKIQDFLQKQEYKTLEYNLTTTEVVMENVTAFWEEGFGELFEKAKQNNNNR
KTSNGDDSLFFSNFSLLGTPVLKDINFKIERGQLLAVAGSTGAGKTSLLMVIMGELEPSE
GKIKHSGRISFCSQFSWIMPGTIKENIIFGVSYDEYRYRSVIKACQLEEDISKFAEKDNIVL
GEGGITLSGGQRARISLARAVYKDADLYLLDSPFGYLDVLTEKEIFESCVCKLMANKTRI
LVTSKMEHLKKADKILILHEGSSYFYGTFSELQNLQPDFSSKLMGCDSFDQFSAERRNSI
LTETLHRFSLEGDAPVSWTETKKQSFKQTGEFGEKRKNSILNPINSIRKFSIVQKTPLQMN
GIEEDSDEPLERRLSLVPDSEQGEAILPRISVISTGPTLQARRRQSVLNLMTHSVNQGQNI
HRKTTASTRKVSLAPQANLTELDIYSRRLSQETGLEISEEINEEDLKECFFDDMESIPAVT
TWNTYLRYITVHKSLIFVLIWCLVIFLAEVAASLVVLWLLGNTPLQDKGNSTHSRNNSY
AVIITSTSSYYVFYIYVGVADTLLAMGFFRGLPLVHTLITVSKILHHKMLHSVLQAPMST
LNTLKAGGILNRFSKDIAILDDLLPLTIFDFIQLLLIVIGAIAVVAVLQPYIFVATVPVIVAF
IMLRAYFLQTSQQLKQLESEGRSPIFTHLVTSLKGLWTLRAFGRQPYFETLFHKALNLHT
ANWFLYLSTLRWFQMRIEMIFVIFFIAVTFISILTTGEGEGRVGIILTLAMNIMSTLQWAV
NSSIDVDSLMRSVSRVFKFIDMPTEGKPTKSTKPYKNGQLSKVMIIENSHVKKDDIWPSG
GQMTVKDLTAKYTEGGNAILENISFSISPGQRVGLLGRTGSGKSTLLSAFLRLLNTEGEI
QIDGVSWDSITLQQWRKAFGVIPQKVFIFSGTFRKNLDPYEQWSDQEIWKVADEVGLRS
VIEQFPGKLDFVLVDGGCVLSHGHKQLMCLARSVLSKAKILLLDEPSAHLDPVTYQIIRR
TLKQAFADCTVILCEHRIEAMLECQQFLVIEENKVRQYDSIQKLLNERSLFRQAISPSDR
VKLFPHRNSSKCKSKPQIAALKEETEEEVQDTRL.

In an aspect, a disclosed encoded CFTR can comprise a sequence having at least least 40%, 50%, 60%, 70%, 80%, 85%, 86%, 87%, 88%, 89%, 90%, 91%, 92%, 93%, 94%, 95%, 96%, 97%, 98%, 99% identity to the sequence set forth in SEQ ID NO:54.

In an aspect, a disclosed nucleic acid sequence to be trans-spliced can encode DMPK or a portion thereof. In an aspect, the one or more disclosed guide RNA sequences can comprise the sequence set forth in SEQ ID NO:29 or SEQ ID NO:30 or a fragment thereof. DMPK is known to the art (e.g., Gene ID 1760) and this nucleotide sequence can comprise nucleotides 5068-17841 in Accession No. NG009784.1. DMPK is a serine-threonine kinase that is closely related to other kinases that interact with members of the Rho family of small GTPases. Substrates for this enzyme include myogenin, the beta-subunit of the L-type calcium channels, and phospholemman. The 3′ untranslated region of this gene contains 5-38 copies of a CTG trinucleotide repeat. Expansion of this unstable motif to 50-5,000 copies causes myotonic dystrophy type I, which increases in severity with increasing repeat element copy number. Repeat expansion is associated with condensation of local chromatin structure that disrupts the expression of genes in this region. Several alternatively spliced transcript variants of this gene have been described, but the full-length nature of some of these variants has not been determined.

In an aspect, a disclosed encoded DMPK can comprise the following sequence or a fragment thereof.

(SEQ ID NO: 56)
MSAEVRLRRLQQLVLDPGFLGLEPLLDLLLGVHQELGASELAQDKYVAD
FLQWAEPIVVRLKEVRLQRDDFEILKVIGRGAFSEVAVVKMKQTGQVYA
MKIMNKWDMLKRGEVSCFREERDVLVNGDRRWITQLHFAFQDENYLYLV
MEYYVGGDLLTLLSKFGERIPAEMARFYLAEIVMAIDSVHRLGYVHRDI
KPDNILLDRCGHIRLADFGSCLKLRADGTVRSLVAVGTPDYLSPEILQA
VGGGPGTGSYGPECDWWALGVFAYEMFYGQTPFYADSTAETYGKIVHYK
EHLSLPLVDEGVPEEARDFIQRLLCPPETRLGRGGAGDFRTHPFFFGLD
WDGLRDSVPPFTPDFEGATDTCNFDLVEDGLTAMETLSDIREGAPLGVH
LPFVGYSYSCMALRDSEVPGPTPMELEAEQLLEPHVQAPSLEPSVSPQD
ETAEVAVPAAVPAAEAEAEVTLRELQEALEEEVLTRQSLSREMEAIRTD
NQNFASQLREAEARNRDLEAHVRQLQERMELLQAEGATAVTGVPSPRAT
DPPSHLDGPPAVAVGQCPLVGPGPMHRRHLLLPARVPRPGLSEALSLLL
FAVVLSRAAALGCIGLVAHAGQLTAVWRRPGAARAP.

In an aspect, a disclosed encoded DMPK can comprise a sequence having at least 40%, 50%, 60%, 70%, 80%, 85%, 86%, 87%, 88%, 89%, 90%, 91%, 92%, 93%, 94%, 95%, 96%, 97%, 98%, 99% identity to the sequence set forth in SEQ ID NO:56.

In an aspect, a disclosed nucleic acid sequence to be trans-spliced can encode DMD or a portion thereof. In an aspect, the one or more disclosed guide RNA sequences can comprise the sequence set forth in SEQ ID NO:26, SEQ ID NO:27, or SEQ ID NO:28 or a fragment thereof.

In an aspect, a disclosed gene can be DMD (dystrophin). DMD is known to the art (e.g., Gene ID 1756) and this nucleotide sequence can comprise nucleotides 5001-2225382 in Accession No. NG012232.1. DMD spans a genomic range of greater than 2 Mb and encodes a large protein containing an N-terminal actin-binding domain and multiple spectrin repeats. The encoded protein forms a component of the dystrophin-glycoprotein complex (DGC), which bridges the inner cytoskeleton and the extracellular matrix. Deletions, duplications, and point mutations at this gene locus may cause Duchenne muscular dystrophy (DMD), Becker muscular dystrophy (BMD), or cardiomyopathy. Alternative promoter usage and alternative splicing result in numerous distinct transcript variants and protein isoforms for this gene.

In an aspect, a disclosed encoded DMD can comprise the following sequence or a fragment thereof.

(SEQ ID NO: 52)
MLWWEEVEDCYEREDVQKKTFTKWVNAQFSKFGKQHIENLFSDLQDGRRLLDLLEGL
TGQKLPKEKGSTRVHALNNVNKALRVLQNNNVDLVNIGSTDIVDGNHKLTLGLIWNIIL
HWQVKNVMKNIMAGLQQTNSEKILLSWVRQSTRNYPQVNVINFTTSWSDGLALNALI
HSHRPDLFDWNSVVCQQSATQRLEHAFNIARYQLGIEKLLDPEDVDTTYPDKKSILMYI
TSLFQVLPQQVSIEAIQEVEMLPRPPKVTKEEHFQLHHQMHYSQQITVSLAQGYERTSSP
KPRFKSYAYTQAAYVTTSDPTRSPFPSQHLEAPEDKSFGSSLMESEVNLDRYQTALEEV
LSWLLSAEDTLQAQGEISNDVEVVKDQFHTHEGYMMDLTAHQGRVGNILQLGSKLIGT
GKLSEDEETEVQEQMNLLNSRWECLRVASMEKQSNLHRVLMDLQNQKLKELNDWLT
KTEERTRKMEEEPLGPDLEDLKRQVQQHKVLQEDLEQEQVRVNSLTHMVVVVDESSG
DHATAALEEQLKVLGDRWANICRWTEDRWVLLQDILLKWQRLTEEQCLFSAWLSEKE
DAVNKIHTTGFKDQNEMLSSLQKLAVLKADLEKKKQSMGKLYSLKQDLLSTLKNKSV
TQKTEAWLDNFARCWDNLVQKLEKSTAQISQAVTTTQPSLTQTTVMETVTTVTTREQI
LVKHAQEELPPPPPQKKRQITVDSEIRKRLDVDITELHSWITRSEAVLQSPEFAIFRKEGN
FSDLKEKVNAIEREKAEKFRKLQDASRSAQALVEQMVNEGVNADSIKQASEQLNSRWI
EFCQLLSERLNWLEYQNNIIAFYNQLQQLEQMTTTAENWLKIQPTTPSEPTAIKSQLKIC
KDEVNRLSDLQPQIERLKIQSIALKEKGQGPMFLDADFVAFTNHFKQVFSDVQAREKEL
QTIFDTLPPMRYQETMSAIRTWVQQSETKLSIPQLSVTDYEIMEQRLGELQALQSSLQEQ
QSGLYYLSTTVKEMSKKAPSEISRKYQSEFEEIEGRWKKLSSQLVEHCQKLEEQMNKLR
KIQNHIQTLKKWMAEVDVFLKEEWPALGDSEILKKQLKQCRLLVSDIQTIQPSLNSVNE
GGQKIKNEAEPEFASRLETELKELNTQWDHMCQQVYARKEALKGGLEKTVSLQKDLSE
MHEWMTQAEEEYLERDFEYKTPDELQKAVEEMKRAKEEAQQKEAKVKLLTESVNSVI
AQAPPVAQEALKKELETLTTNYQWLCTRLNGKCKTLEEVWACWHELLSYLEKANKW
LNEVEFKLKTTENIPGGAEEISEVLDSLENLMRHSEDNPNQIRILAQTLTDGGVMDELIN
EELETFNSRWRELHEEAVRRQKLLEQSIQSAQETEKSLHLIQESLTFIDKQLAAYIADKV
DAAQMPQEAQKIQSDLTSHEISLEEMKKHNQGKEAAQRVLSQIDVAQKKLQDVSMKF
RLFQKPANFEQRLQESKMILDEVKMHLPALETKSVEQEVVQSQLNHCVNLYKSLSEVK
SEVEMVIKTGRQIVQKKQTENPKELDERVTALKLHYNELGAKVTERKQQLEKCLKLSR
KMRKEMNVLTEWLAATDMELTKRSAVEGMPSNLDSEVAWGKATQKEIEKQKVHLKSI
TEVGEALKTVLGKKETLVEDKLSLLNSNWIAVTSRAEEWLNLLLEYQKHMETFDQNV
DHITKWIIQADTLLDESEKKKPQQKEDVLKRLKAELNDIRPKVDSTRDQAANLMANRG
DHCRKLVEPQISELNHRFAAISHRIKTGKASIPLKELEQFNSDIQKLLEPLEAEIQQGVNL
KEEDFNKDMNEDNEGTVKELLQRGDNLQQRITDERKREEIKIKQQLLQTKHNALKDLR
SQRRKKALEISHQWYQYKRQADDLLKCLDDIEKKLASLPEPRDERKIKEIDRELQKKKE
ELNAVRRQAEGLSEDGAAMAVEPTQIQLSKRWREIESKFAQFRRLNFAQIHTVREETM
MVMTEDMPLEISYVPSTYLTEITHVSQALLEVEQLLNAPDLCAKDFEDLFKQEESLKNIK
DSLQQSSGRIDIIHSKKTAALQSATPVERVKLQEALSQLDFQWEKVNKMYKDRQGRFD
RSVEKWRRFHYDIKIFNQWLTEAEQFLRKTQIPENWEHAKYKWYLKELQDGIGQRQTV
VRTLNATGEEIIQQSSKTDASILQEKLGSLNLRWQEVCKQLSDRKKRLEEQKNILSEFQR
DLNEFVLWLEEADNIASIPLEPGKEQQLKEKLEQVKLLVEELPLRQGILKQLNETGGPVL
VSAPISPEEQDKLENKLKQTNLQWIKVSRALPEKQGEIEAQIKDLGQLEKKLEDLEEQLN
HLLLWLSPIRNQLEIYNQPNQEGPFDVKETEIAVQAKQPDVEEILSKGQHLYKEKPATQP
VKRKLEDLSSEWKAVNRLLQELRAKQPDLAPGLTTIGASPTQTVTLVTQPVVTKETAIS
KLEMPSSLMLEVPALADFNRAWTELTDWLSLLDQVIKSQRVMVGDLEDINEMIIKQKA
TMQDLEQRRPQLEELITAAQNLKNKTSNQEARTIITDRIERIQNQWDEVQEHLQNRRQQ
LNEMLKDSTQWLEAKEEAEQVLGQARAKLESWKEGPYTVDAIQKKITETKQLAKDLR
QWQTNVDVANDLALKLLRDYSADDTRKVHMITENINASWRSIHKRVSEREAALEETHR
LLQQFPLDLEKFLAWLTEAETTANVLQDATRKERLLEDSKGVKELMKQWQDLQGEIEA
HTDVYHNLDENSQKILRSLEGSDDAVLLQRRLDNMNFKWSELRKKSLNIRSHLEASSD
QWKRLHLSLQELLVWLQLKDDELSRQAPIGGDFPAVQKQNDVHRAFKRELKTKEPVIM
STLETVRIFLTEQPLEGLEKLYQEPRELPPEERAQNVTRLLRKQAEEVNTEWEKLNLHSA
DWQRKIDETLERLQELQEATDELDLKLRQAEVIKGSWQPVGDLLIDSLQDHLEKVKAL
RGEIAPLKENVSHVNDLARQLTTLGIQLSPYNLSTLEDLNTRWKLLQVAVEDRVRQLHE
AHRDFGPASQHELSTSVQGPWERAISPNKVPYYINHETQTTCWDHPKMTELYQSLADL
NNVRFSAYRTAMKLRRLQKALCLDLLSLSAACDALDQHNLKQNDQPMDILQIINCLTTI
YDRLEQEHNNLVNVPLCVDMCLNWLLNVYDTGRTGRIRVLSFKTGIISLCKAHLEDKY
RYLFKQVASSTGFCDQRRLGLLLHDSIQIPRQLGEVASFGGSNIEPSVRSCFQFANNKPEI
EAALFLDWMRLEPQSMVWLPVLHRVAAAETAKHQAKCNICKECPIIGFRYRSLKHFNY
DICQSCFFSGRVAKGHKMHYPMVEYCTPTTSGEDVRDFAKVLKNKFRTKRYFAKHPR
MGYLPVQTVLEGDNMETPVTLINFWPVDSAPASSPQLSHDDTHSRIEHYASRLAEMENS
NGSYLNDSISPNESIDDEHLLIQHYCQSLNQDSPLSQPRSPAQILISLESEERGELERILADL
EEENRNLQAEYDRLKQQHEHKGLSPLPSPPEMMPTSPQSPRDAELIAEAKLLRQHKGRL
EARMQILEDHNKQLESQLHRLRQLLEQPQAEAKVNGTTVSSPSTSLQRSDSSQPMLLRV
VGSQTSDSMGEEDLLSPPQDTSTGLEEVMEQLNNSFPSSRGRNTPGKPMREDTM.

In an aspect, a disclosed encoded DMD can comprise a sequence having at least 40%, 50%, 60%, 70%, 80%, 85%, 86%, 87%, 88%, 89%, 90%, 91%, 92%, 93%, 94%, 95%, 96%, 97%, 98%, 99% identity to the sequence set forth in SEQ ID NO:52.

In an aspect, a disclosed nucleic acid sequence to be trans-spliced can encode LRRK2 or a portion thereof. LRRK2 is known to the art (e.g., Gene ID 120892) and this nucleotide sequence can comprise nucleotides 5001-149275 in Accession No. NG011709.1. LRRK2 is a member of the leucine-rich repeat kinase family and encodes a protein with an ankryin repeat region, a leucine-rich repeat (LRR) domain, a kinase domain, a DFG-like motif, a RAS domain, a GTPase domain, a MLK-like domain, and a WD40 domain. The protein is present largely in the cytoplasm but also associates with the mitochondrial outer membrane. Mutations in this gene have been associated with Parkinson's disease.

In an aspect, a disclosed encoded LRRK2 can comprise the following sequence or a fragment thereof.

(SEQ ID NO: 53)
MASGSCQGCEEDEETLKKLIVRLNNVQEGKQIETLVQILEDLLVFTYSERASKLFQGKNI
HVPLLIVLDSYMRVASVQQVGWSLLCKLIEVCPGTMQSLMGPQDVGNDWEVLGVHQL
ILKMLTVHNASVNLSVIGLKTLDLLLTSGKITLLILDEESDIFMLIFDAMHSFPANDEVQK
LGCKALHVLFERVSEEQLTEFVENKDYMILLSALTNFKDEEEIVLHVLHCLHSLAIPCNN
VEVLMSGNVRCYNIVVEAMKAFPMSERIQEVSCCLLHRLTLGNFFNILVLNEVHEFVVK
AVQQYPENAALQISALSCLALLTETIFLNQDLEEKNENQENDDEGEEDKLFWLEACYKA
LTWHRKNKHVQEAACWALNNLLMYQNSLHEKIGDEDGHFPAHREVMLSMLMHSSSK
EVFQASANALSTLLEQNVNFRKILLSKGIHLNVLELMQKHIHSPEVAESGCKMLNHLFE
GSNTSLDIMAAVVPKILTVMKRHETSLPVQLEALRAILHFIVPGMPEESREDTEFHHKLN
MVKKQCFKNDIHKLVLAALNRFIGNPGIQKCGLKVISSIVHFPDALEMLSLEGAMDSVL
HTLQMYPDDQEIQCLGLSLIGYLITKKNVFIGTGHLLAKILVSSLYRFKDVAEIQTKGFQ
TILAILKLSASFSKLLVHHSFDLVIFHQMSSNIMEQKDQQFLNLCCKCFAKVAMDDYLK
NVMLERACDQNNSIMVECLLLLGADANQAKEGSSLICQVCEKESSPKLVELLLNSGSRE
QDVRKALTISIGKGDSQIISLLLRRLALDVANNSICLGGFCIGKVEPSWLGPLFPDKTSNL
RKQTNIASTLARMVIRYQMKSAVEEGTASGSDGNFSEDVLSKFDEWTFIPDSSMDSVFA
QSDDLDSEGSEGSFLVKKKSNSISVGEFYRDAVLQRCSPNLQRHSNSLGPIFDHEDLLKR
KRKILSSDDSLRSSKLQSHMRHSDSISSLASEREYITSLDLSANELRDIDALSQKCCISVHL
EHLEKLELHQNALTSFPQQLCETLKSLTHLDLHSNKFTSFPSYLLKMSCIANLDVSRNDI
GPSVVLDPTVKCPTLKQFNLSYNQLSFVPENLTDVVEKLEQLILEGNKISGICSPLRLKEL
KILNLSKNHISSLSENFLEACPKVESFSARMNFLAAMPFLPPSMTILKLSQNKFSCIPEAIL
NLPHLRSLDMSSNDIQYLPGPAHWKSLNLRELLFSHNQISILDLSEKAYLWSRVEKLHLS
HNKLKEIPPEIGCLENLTSLDVSYNLELRSFPNEMGKLSKIWDLPLDELHLNFDFKHIGC
KAKDIIRFLQQRLKKAVPYNRMKLMIVGNTGSGKTTLLQQLMKTKKSDLGMQSATVGI
DVKDWPIQIRDKRKRDLVLNVWDFAGREEFYSTHPHFMTQRALYLAVYDLSKGQAEV
DAMKPWLFNIKARASSSPVILVGTHLDVSDEKQRKACMSKITKELLNKRGFPAIRDYHF
VNATEESDALAKLRKTIINESLNFKIRDQLVVGQLIPDCYVELEKIILSERKNVPIEFPVID
RKRLLQLVRENQLQLDENELPHAVHFLNESGVLLHFQDPALQLSDLYFVEPKWLCKIM
AQILTVKVEGCPKHPKGIISRRDVEKFLSKKRKFPKNYMSQYFKLLEKFQIALPIGEEYLL
VPSSLSDHRPVIELPHCENSEIIIRLYEMPYFPMGFWSRLINRLLEISPYMLSGRERALRPN
RMYWRQGIYLNWSPEAYCLVGSEVLDNHPESFLKITVPSCRKGCILLGQVVDHIDSLME
EWFPGLLEIDICGEGETLLKKWALYSFNDGEEHQKILLDDLMKKAEEGDLLVNPDQPRL
TIPISQIAPDLILADLPRNIMLNNDELEFEQAPEFLLGDGSFGSVYRAAYEGEEVAVKIFN
KHTSLRLLRQELVVLCHLHHPSLISLLAAGIRPRMLVMELASKGSLDRLLQQDKASLTR
TLQHRIALHVADGLRYLHSAMIIYRDLKPHNVLLFTLYPNAAIIAKIADYGIAQYCCRM
GIKTSEGTPGFRAPEVARGNVIYNQQADVYSFGLLLYDILTTGGRIVEGLKFPNEFDELEI
QGKLPDPVKEYGCAPWPMVEKLIKQCLKENPQERPTSAQVFDILNSAELVCLTRRILLP
KNVIVECMVATHHNSRNASIWLGCGHTDRGQLSFLDLNTEGYTSEEVADSRILCLALV
HLPVEKESWIVSGTQSGTLLVINTEDGKKRHTLEKMTDSVTCLYCNSFSKQSKQKNFLL
VGTADGKLAIFEDKTVKLKGAAPLKILNIGNVSTPLMCLSESTNSTERNVMWGGCGTKI
FSFSNDFTIQKLIETRTSQLFSYAAFSDSNIITVVVDTALYIAKQNSPVVEVWDKKTEKLC
GLIDCVHFLREVMVKENKESKHKMSYSGRVKTLCLQKNTALWIGTGGGHILLLDLSTR
RLIRVIYNFCNSVRVMMTAQLGSLKNVMLVLGYNRKNTEGTQKQKEIQSCLTVWDINL
PHEVQNLEKHIEVRKELAEKMRRTSVE.

In an aspect, a disclosed encoded LRRK2 can comprise a sequence having at least 40%, 50%, 60%, 70%, 80%, 85%, 86%, 87%, 88%, 89%, 90%, 91%, 92%, 93%, 94%, 95%, 96%, 97%, 98%, 99% identity to the sequence set forth in SEQ ID NO:53.

In an aspect, a disclosed nucleic acid sequence to be trans-spliced can encode the protein or a portion thereof (such as, for example, Exon 1 or Exon 4, etc.) associated with the following genes: ABCA1, ABCA12, ABCA13, ABCA2, ABCA3, ABCA4, ABCA5, ABCC1, ABCC2, ABCC6, ABCC8, ABCC9, ACAN, ADAMTS13, ADCY10, ADGRV1, AGL, AGRN, AHDC1, ALK, ALMS1, ALPK3, ALS2, ANAPC1, ANK1, ANK2, ANK3, ANKRD11, ANKRD26, APC, APC2, APOB, ARFGEF2, ARHGAP31, ARHGEF10, ARHGEF18, ARID1A, ARIDIB, ARID2, ASH1L, ASPM, ASXL1, ASXL2, ASXL3, ATM, ATP7A, ATP7B, ATR, ATRX, BAZ1A, BAZ2B, BCOR, BCORL1, BDP1, BLM, BPTF, BRCA1, BRCA2, BRD4, BRWD3, C2CD3, C3, C5, CACNA1A, CACNA1B, CACNA1C, CACNA1D, CACNA1E, CACNA1F, CACNA1G, CACNA1H, CACNA1S, CAD, CAMTA1, CARMIL2, CC2D2A, CCDC88A, CCDC88C, CCNB3, CDH23, CDK13, CDK5RAP2, CELSR1, CEMIP2, CENPE, CENPF, CENPJ, CEP152, CEP164, CEP250, CEP290, CFAP43, CFAP44, CFAP65, CFTR/ABCC7, CHD1, CHD2, CHD3, CHD4, CHD7, CHD8, CIC, CIT, CLIP1, CLTC, CNOT1, CNTNAP1, COL11A1, COL11A2, COL12A1, COL17A1, COL18A1, COL1A1, COL1A2, COL27A1, COL2A1, COL3A1, COL4A1, COL4A2, COL4A3, COL4A4, COL4A5, COL4A6, COL5A1, COL5A2, COL6A3, COL7A1, CPAMD8, CPLANEl, CPS1, CPSF1, CRB1, CREBBP, CUBN, CUL7, CUX1, DCC, DCHS1, DEPDC5, DICERI, DIP2B, DLC1, DMD, DMXL2, DNAH1, DNAH11, DNAH17, DNAH2, DNAH5, DNAH7, DNAH8, DNAH9, DNMBP, DNMT1, DOCK2, DOCK3, DOCK6, DOCK7, DOCK8, DSCAM, DSP, DST, DUOX2, DYNCIHI, DYNC2H1, DYSF, EIF2AK4, EP300, EPG5, ERCC6, ERCC6L2, EXPH5, EYS, F5, F8, FANCA, FANCD2, FANCM, FAT1, FAT4, FBN1, FBN2, FLG, FLG2, FLNA, FLNB, FLNC, FLT4, FMN2, FN1, FRAS1, FREM1, FREM2, FSIP2, FYCO1, GLI2, GLI3, GPR179, GREBIL, GRIN2A, GRIN2B, GRIN2D, HCFC1, HECW2, HERC1, HERC2, HFM1, HIVEP1, HIVEP2, HMCN1, HSPG2, HTT, HUWE1, HYDIN, IFT140, IFT172, IGF1R, IGF2R, IGSF1, INSR, INTS1, IQSEC2, ITGB4, ITPR1, ITPR2, JMJD1C, KALRN, KANK1, KAT6A, KAT6B, KDM3B, KDM5B, KDM5C, KDM6A, KDM6B, KDR, KIAA0586, KIAA1109, KIAA1549, KIDINS220, KIF14, KIF1A, KIF1B, KIF21A, KIF26B, KIF7, KMT2A, KMT2B, KMT2C, KMT2D, KMT2E, KNL1, LAMA1, LAMA2, LAMA3, LAMA4, LAMA5, LAMB1, LAMB2, LAMC3, LCT, LOXHD1, LPA, LRBA, LRP1, LRP2, LRP4, LRP5, LRP6, LRPPRC, LRRK1, LRRK2, LTBP2, LTBP4, LYST, MACF1, MADD, MAGI2, MAP1B, MAP3K1, MAPK8IP3, MAPKBP1, MAST1, MBD5, MCM3AP, MED12, MED12L, MED13, MED13L, MED23, MEGF8, MET, MLH3, MPDZ, MSH6, MTOR, MYH10, MYH11, MYH14, MYH2, MYH3, MYH6, MYH7, MYH7B, MYH8, MYH9, MYLK, MYO15A, MYO18B, MYO3A, MYO5A, MYO5B, MYO7A, MYO9A, NALCN, NBAS, NBEA, NBEAL2, NCAPD2, NCAPD3, NEB, NEXMIF, NEXMIF, NF1, NFASC, NHS, NIN, NIPBL, NLRP1, NOTCH1, NOTCH2, NOTCH3, NPHP4, NRXN1, NRXN3, NSD1, NSD2, NUP155, NUP188, NUP205, OBSCN, OBSL1, OTOF, OTOG, OTOGL, PARD3, PBRM1, PCDH15, PCLO, PCNT, PHIP, PI4KA, PIEZO1, PIEZO2, PIK3C2A, PIKFYVE, PKD1, PKD1L1, PKHD1, PLCE1, PLEC, PLEKHG2, PNPLA6, POGZ, POLA1, POLE, POLR1A, POLR2A, POLR3A, PRG4, PRKDC, PRPF8, PRR12, PRX, PTCH1, PTPN23, PTPRF, PTPRJ, PTPRQ, PXDN, QRICH2, RAB3GAP2, RAIl, RALGAPA1, RANBP2, RB1CC1, RELN, RERE, REV3L, RIC1, RIMS1, RIMS2, RNF213, ROBO1, ROBO2, ROBO3, ROS1, RP1, RP1L1, RTTN, RUSC2, RYR1, RYR2, SACS, SAMD9, SAMD9L, SBF2, SCAPER, SCN10A, SCN11A, SCN1A, SCN2A, SCN3A, SCN4A, SCN5A, SCN8A, SCN9A, SETBP1, SETD1A, SETD1B, SETD2, SETD5, SETX, SHANK2, SHANK3, SHROOM4, SI, SIPA1L3, SLIT2, SLX4, SMARCA2, SMARCA4, SMCHD1, SNRNP200, SON, SPEF2, SPEG, SPG11, SPTA1, SPTAN1, SPTB, SPTBN2, SPTBN4, SRCAP, STRC, SVIL, SYNE1, SYNGAP1, SYNJ1, SZT2, TAF1, TANC2, TCF20, TCOF1, TDRD9, TECPR2, TECTA, TENM3, TENM4, TET3, TEX14, TEX15, TG, THOC2, TMEM94, TNC, TNIK, TNR, TNRC6B, TNXB, TOGARAMI, TONSL, TRIO, TRIOBP, TRIP11, TRIP12, TRPM1, TRPM6, TRPM7, TRRAP, TSC2, TTC37, TTN, TUBGCP6, UBR1, UNC80, USH2A, USP9X, VCAN, VPS13A, VPS13B, VPS13C, VPS13D, VWF, WDFY3, WDR19, WDR62, WDR81, WNK1, WRN, ZFHX2, ZFYVE26, ZNF142, ZNF292, ZNF335, ZNF407, ZNF462, ZNF469, or a portion thereof.

In an aspect, a disclosed nucleic acid sequence to be trans-spliced can encode a gene or a portion thereof (e.g., a specific exon such as an exon having a mutation) with a gene product that is directly or indirectly linked to one or more genetic diseases. Such genes include but are not limited to the following: dystrophin including mini- and micro-dystrophins (DMD); titin (TTN); titin cap (TCAP) α-sarcoglycan (SGCA), β-sarcoglycan (SGCB), γ-sarcoglycan (SGCG) or 6-sarcoglycan (SGCD); alpha-1-antitrypsin (A1-AT); myosin heavy chain 6 (MYH6); myosin heavy chain 7 (MYH7); myosin heavy chain 11 (MYH11); myosin light chain 2 (ML2); myosin light chain 3 (ML3); myosin light chain kinase 2 (MYLK2); myosin binding protein C (MYBPC3); desmin (DES); dynamin 2 (DNM2); laminin α2 (LAMA2); lamin A/C (LMNA); lamin B (LMNB); lamin B receptor (LBR); dysferlin (DYSF); emerin (EMD); insulin; blood clotting factors, including but not limited to, factor VIII and factor IX; erythropoietin (EPO); lipoprotein lipase (LPL); sarcoplasmic reticulum Ca2++-ATPase (SERCA2A), S100 calcium binding protein A1 (S100A1); myotubularin (MTM); DM1 protein kinase (DMPK); glycogen phosphorylase L (PYGL); glycogen phosphorylase, muscle associated (PYGM); glycogen synthase 1 (GYS1); glycogen synthase 2 (GYS2); α-galactosidase A (GLA); α-N-acetylgalactosaminidase (NAGA); acid α-glucosidase (GAA), sphingomyelinase phosphodiesterase 1 (SMPD1); lysosomal acid lipase (LIPA); collagen type I α1 chain (COL1A1); collagen type I α2 chain (COL1A2); collagen type III α1 chain (COL3A1); collagen type V al chain (COL5A1); collagen type V α2 chain (COL5A2); collagen type VI α1 chain (COL6A1); collagen type VI α2 chain (COL6A2); collagen type VI 3 chain (COL6A3); procollagen-lysine 2-oxoglutarate 5-dioxygenase (PLOD1); lysosomal acid lipase (LIPA); frataxin (FXN); myostatin (MSTN); β-N-acetyl hexosaminidase A (HEXA); β-N-acetylhexosaminidase B (HEXB); β-glucocerebrosidase (GBA); adenosine monophosphate deaminase 1 (AMPD1); β-globin (HBB); iduronidase (IDUA); iduronate 2-sulfate (IDS); troponin 1 (TNNI3); troponin T2 (TNNT2); troponin C (TNNC1); tropomyosin 1 (TPM1); tropomyosin 3 (TPM3); N-acetyl-α-glucosaminidase (NAGLU); N-sulfoglucosamine sulfohydrolase (SGSH); heparan-α-glucosaminide N-acetyltransferase (HGSNAT); integrin a 7 (IGTA7); integrin a 9 (IGTA9); glucosamine(N-acetyl)-6-sulfatase (GNS); galactosamine(N-acetyl)-6-sulfatase (GALNS); β-galactosidase (GLB1); β-glucuronidase (GUSB); hyaluronoglucosaminidase 1 (HYAL1); acid ceramidase (ASAHI); galactosylcermidase (GALC); cathepsin A (CTSA); cathepsin D (CTSA); cathepsin K (CTSK); GM2 ganglioside activator (GM2A); arylsulfatase A (ARSA); arylsulfatase B (ARSB); formylglycine-generating enzyme (SUMFI); neuraminidase 1 (NEU1); N-acetylglucosamine-1-phosphate transferase a (GNPTA); N-acetylglucosamine-1-phosphate transferase β (GNPTB); N-acetylglucosamine-1-phosphate transferase γ (GNPTG); mucolipin-1 (MCOLN1); NPC intracellular transporter 1 (NPC1); NPC intracellular transporter 2 (NPC2); ceroid lipofuscinosis 5 (CLN5); ceroid lipofuscinosis 6 (CLN6); ceroid lipofuscinosis 8 (CLN8); palmitoyl protein thioesterase 1 (PPT1); tripeptidyl peptidase 1 (TPP1); battenin (CLN3); DNAJ heat shock protein family 40 member C5 (DNAJC5); major facilitator superfamily domain containing 8 (MFSD8); mannosidase a class 2B member 1 (MAN2B1); mannosidase R (MANBA); aspartylglucosaminidase (AGA); α-L-fucosidase (FUCA1); cystinosin, lysosomal cysteine transporter (CTNS); sialin; solute carrier family 2 member 10 (SLC2A10); solute carrier family 17 member 5 (SLC17A5); solute carrier family 6 member 19 (SLC6A19); solute carrier family 22 member 5 (SLC22A5); solute carrier family 37 member 4 (SLC37A4); lysosomal associated membrane protein 2 (LAMP2); sodium voltage-gated channel α subunit 4 (SCN4A); sodium voltage-gated channel R subunit 4 (SCN4B); sodium voltage-gated channel α subunit 5 (SCN5A); sodium voltage-gated channel α subunit 4 (SCN4A); calcium voltage-gated channel subunit α1c (CACNA1C); calcium voltage-gated channel subunit α1s (CACNA1S); phosphoglycerate kinase 1 (PGK1); phosphoglycerate mutase 2 (PGAM2); amylo-α-1,6-glucosidase,4-α-glucanotransferase (AGL); potassium voltage-gated channel ISK-related subfamily member 1 (KCNE1); potassium voltage-gated channel ISK-related subfamily member 2 (KCNE2); potassium voltage-gated channel subfamily J member 2 (KCNJ2); potassium voltage-gated channel subfamily J member 5 (KCNJ5); potassium voltage-gated channel subfamily H member 2 (KCNH2); potassium voltage-gated channel KQT-like subfamily member 1 (KCNQ1); hyperpolarization-activated cyclic nucleotide-gated potassium channel 4 (HCN4); chloride voltage-gated channel 1 (CLCN1); camitine palmitoyltransferase 1A (CPT1A); ryanodine receptor 1 (RYR1); ryanodine receptor 2 (RYR2); bridging integrator 1 (BIN1); LARGE xylosyl- and glucuronyltransferase 1 (LARGEl); docking protein 7 (DOK7); fukutin (FKTN); fukutin related protein (FKRP); selenoprotein N (SELENON); protein O-mannosyltransferase 1 (POMT1); protein β-mannosyltransferase 2 (POMT2); protein O-linked mannose N-acetylglucosaminyltransferase 1 (POMGNT1); protein O-linked mannose N-acetylglucosaminyltransferase 2 (POMGNT2); protein-O-mannose kinase (POMK); isoprenoid synthase domain containing (ISPD); plectin (PLEC); cholinergic receptor nicotinic epsilon subunit (CHRNE); choline O-acetyltransferase (CHAT); choline kinase β (CHKB); collagen like tail subunit of asymmetric acetylcholinesterase (COLQ); receptor associated protein of the synapse (RAPSN); four and a half LIM domains 1 (FHL1); β-1,4-glucuronyltransferase 1 (B4GAT1); β-1,3-N-acetylgalactosaminyltransferase 2 (B3GALNT2); dystroglycan 1 (DAGI); transmembrane protein 5 (TMEM5); transmembrane protein 43 (TMEM43); SECIS binding protein 2 (SECISBP2); glucosamine (UDP-N-acetyl)-2-epimerase/N-acetylmannosamine kinase (GNE); anoctamin 5 (ANO5); structural maintenance of chromosomes flexible hinge domain containing 1 (SMCHD1); lactate dehydrogenase A (LDHA); lactate dehydrogenase B (LHDB); calpain 3 (CAPN3); caveolin 3 (CAV3); tripartite motif containing 32 (TRIM32); CCHC-type zinc finger nucleic acid binding protein (CNBP); nebulin (NEB); actin, α1, skeletal muscle (ACTA1); actin, α1, cardiac muscle (ACTC1); actinin α2 (ACTN2); poly(A)-binding protein nuclear 1 (PABPN1); LEM domain-containing protein 3 (LEMD3); zinc metalloproteinase STE24 (ZMPSTE24); microsomal triglyceride transfer protein (MTTP); cholinergic receptor nicotinic al subunit (CHRNA1); cholinergic receptor nicotinic α2 subunit (CHRNA2); cholinergic receptor nicotinic β3 subunit (CHRNA3); cholinergic receptor nicotinic β4 subunit (CHRNA4); cholinergic receptor nicotinic β5 subunit (CHRNA5); cholinergic receptor nicotinic α6 subunit (CHRNA6); cholinergic receptor nicotinic c7 subunit (CHRNA7); cholinergic receptor nicotinic α8 subunit (CHRNA8); cholinergic receptor nicotinic α9 subunit (CHRNA9); cholinergic receptor nicotinic α10 subunit (CHRNA10); cholinergic receptor nicotinic 31 subunit (CHRNB1); cholinergic receptor nicotinic β2 subunit (CHRNB2); cholinergic receptor nicotinic β3 subunit (CHRNB3); cholinergic receptor nicotinic β4 subunit (CHRNB4); cholinergic receptor nicotinic γ subunit (CHRNG1); cholinergic receptor nicotinic a subunit (CHRND); cholinergic receptor nicotinic E subunit (CHRNE1); ATP binding cassette subfamily A member 1 (ABCA1); ATP binding cassette subfamily C member 6 (ABCC6); ATP binding cassette subfamily C member 9 (ABCC9); ATP binding cassette subfamily D member 1 (ABCD1); ATPase sarcoplasmic/endoplasmic reticulum Ca2+ transporting 1 (ATP2A1); ATM serine/threonine kinase (ATM); a tocopherol transferase protein (TTPA); kinesin family member 21A (KIF21A); paired-like homeobox 2a (PHOX2A); heparan sulfate proteoglycan 2 (HSPG2); stromal interaction molecule 1 (STIM1); notch 1 (NOTCH1); notch 3 (NOTCH3); dystrobrevin a (DTNA); protein kinase AMP-activated, noncatalytic γ2 (PRKAG2); cysteine- and glycine-rich protein 3 (CSRP3); viniculin (VCL); myozenin 2 (MyoZ2); myopalladin (MYPN); junctophilin 2 (JPH2); phospholamban (PLN); calreticulin 3 (CALR3); nexilin F-actin-binding protein (NEXN); LIM domain binding 3 (LDB3); eyes absent 4 (EYA4); huntingtin (HTT); androgen receptor (AR); protein tyrosine phosphate non-receptor type 11 (PTPN11); junction plakoglobin (JUP); desmoplakin (DSP); plakophilin 2 (PKP2); desmoglein 2 (DSG2); desmocollin 2 (DSC2); catenin α3 (CTNNA3); NK2 homeobox 5 (NKX2-5); A-kinase anchor protein 9 (AKAP9); A-kinase anchor protein 10 (AKAP10); guanine nucleotide-binding protein α-inhibiting activity polypeptide 2 (GNA12); ankyrin 2 (ANK2); syntrophin α-1 (SNTAT); calmodulin 1 (CALM1); calmodulin 2 (CALM2); HTRA serine peptidase 1 (HTRA1); fibrillin 1 (FBN1); fibrillin 2 (FBN2); xylosyltransferase 1 (XYLT1); xylosyltransferase 2 (XYLT2); tafazzin (TAZ); homogentisate 1,2-dioxygenase (HGD); glucose-6-phosphatase catalytic subunit (G6PC); 1,4-alpha-glucan enzyme 1 (GBE1); phosphofructokinase, muscle (PFKM); phosphorylase kinase regulatory subunit alpha 1 (PHKA1); phosphorylase kinase regulatory subunit alpha 2 (PHKA2); phosphorylase kinase regulatory subunit beta (PHKB); phosphorylase kinase catalytic subunit gamma 2 (PHKG2); phosphoglycerate mutase 2 (PGAM2); cystathionine-beta-synthase (CBS); methylenetetrahydrofolate reductase (MTHFR); 5-methyltetrahydrofolate-homocysteine methyltransferase (MTR); 5-methyltetrahydrofolate-homocysteine methyltransferase reductase (MTRR); methylmalonic aciduria and homocystinuria, cblD type (MMADHC); mitochondrial DNA, including, but not limited to mitochondrially encoded NADH:ubiquinone oxidoreductase core subunit 1 (MT-ND1); mitochondrially encoded NADH:ubiquinone oxidoreductase core subunit 5 (MT-ND5); mitochondrially encoded tRNA glutamic acid (MT-TE); mitochondrially encoded tRNA histadine (MT-TH); mitochondrially encoded tRNA leucine 1 (MT-TL1); mitochondrially encoded tRNA lysine (MT-TK); mitochondrially encoded tRNA serine 1 (MT-TS1); mitochondrially encoded tRNA valine (MT-TV); mitogen-activated protein kinase 1 (MAP2K1); B-Raf proto-oncogene, serine/threonine kinase (BRAF); raf-1 proto-oncogene, serine/threonine kinase (RAF1); growth factors, including, but not limited to insulin growth factor 1 (IGF-1); transforming growth factor β3 (TGFβ3); transforming growth factor β receptor, type I (TGFβ1); transforming growth factor β receptor, type II (TGFβ2), fibroblast growth factor 2 (FGF2), fibroblast growth factor 4 (FGF4), vascular endothelial growth factor A (VEGF-A), vascular endothelial growth factor B (VEGF-B); vascular endothelial growth factor C (VEGF-C), vascular endothelial growth factor D (VEGF-D), vascular endothelial growth factor receptor 1 (VEGFR1), and vascular endothelial growth factor receptor 2 (VEGFR2); interleukins; immunoadhesins; cytokines; and antibodies.

In an aspect, a disclosed nucleic acid sequence to be trans-spliced can be CpG depleted and codon-optimized for expression in a human cell. In an aspect, “CpG-free” can mean completely free of CpGs or partially free of CpGs. In an aspect, “CpG-free” can mean “CpG-depleted”. In an aspect, “CpG-depleted” can mean “CpG-free”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art.

In an aspect, a disclosed nucleic acid sequence to be trans-spliced can encode an RNA. In an aspect, a disclosed encoded RNA can comprise ribosomal RNA (rRNA), transfer RNA (tRNA), heterogeneous nuclear RNA (hnRNA), small nuclear RNA (snRNA), small nucleolar RNA (snoRNA), micro RNA (miRNA), Piwi-interacting RNA (piRNA), small interfering RNA (siRNA), short hairpin RNA (shRNA), singe guide RNA (sgRNA), non-coding RNA (ncRNA), long non-coding RNA (lncRNA), 7SL, Xist, short enhancer RNA (eRNA), circular RNA, intergenic RNA, or any combination thereof. In an aspect, a disclosed encoded RNA can comprise lncRNA, siRNA, shRNA, sgRNA, circular RNA, snoRNA, miRNA, or any combination thereof. In an aspect, a disclosed encoded RNA can comprise a functional non-coding RNA element.

In an aspect, a disclosed promoter for the one or more disclosed guide RNA sequences can be tissue-specific or ubiquitous and can be constitutive or inducible, depending on the pattern of the gene expression desired. A promoter can be native or foreign and can be a natural or a synthetic sequence. By foreign, it is intended that the transcriptional initiation region is not found in the wild-type host into which the transcriptional initiation region is introduced. In an aspect, a disclosed promoter can be a promoter/enhancer. In an aspect, a disclosed promoter for the one or more disclosed guide RNA sequences can be an endogenous promoter. In an aspect, a disclosed endogenous promoter can be an endogenous promoter/enhancer. In an aspect, a disclosed endogenous promoter or a disclosed endogenous promoter/enhancer can generally be obtained from a non-coding region upstream of a transcription initiation site of a gene of interest. In an aspect, a disclosed endogenous promoter or a disclosed endogenous promoter/enhancer can be used for constitutive and efficient expression of a disclosed gene. In an aspect, a disclosed promoter for the one or more disclosed guide RNA sequences can be a CMV promoter or a CMV promoter/enhancer. CMV promoters and CMV promoters/enhancers are well known to the art. In an aspect, a disclosed promoter for the one or more disclosed guide RNA sequences can be any eukaryotic RNA polymerase II promoter.

Disclosed herein is an expression cassette, comprising an isolated nucleic acid molecule comprising a nucleic acid sequence to be trans-spliced to a target endogenous pre-mRNA; a 5′ hemi intron linked to the nucleic acid sequence to be trans-spliced; one or more guide RNA sequences; a promoter operably linked to the one or more guide RNA sequences; and a nucleic acid sequence encoding an RNA binding protein.

Disclosed herein is an isolated nucleic acid molecule, comprising a nucleic acid sequence encoding a catalytically inactive PspdCas13b, a promoter operably linked to the nucleic acid sequence encoding the catalytically inactive PspdCas13b; and a polyadenylation signal.

In an aspect, a disclosed Cas13d can further comprise one or more other agents or domains (e.g., is a fusion protein), such as one or more subcellular localization signals, one or more effector domains, or any combinations thereof.

In an aspect, a disclosed promoter for a catalytically inactive PspdCas13b can be tissue-specific or ubiquitous and can be constitutive or inducible, depending on the pattern of the gene expression desired. A promoter can be native or foreign and can be a natural or a synthetic sequence. By foreign, it is intended that the transcriptional initiation region is not found in the wild-type host into which the transcriptional initiation region is introduced. In an aspect, a disclosed promoter for a catalytically inactive PspdCas13b can be a promoter/enhancer. In an aspect, a disclosed promoter can be an endogenous promoter. In an aspect, a disclosed endogenous promoter can be an endogenous promoter/enhancer. In an aspect, a disclosed endogenous promoter or a disclosed endogenous promoter/enhancer can generally be obtained from a non-coding region upstream of a transcription initiation site of a gene of interest. In an aspect, a disclosed endogenous promoter or a disclosed endogenous promoter/enhancer can be used for constitutive and efficient expression of a disclosed gene. In an aspect, a disclosed promoter for a catalytically inactive PspdCas13b can be a CMV promoter or a CMV promoter/enhancer. CMV promoters and CMV promoters/enhancers are well known to the art. In an aspect, a disclosed promoter for the one or more disclosed guide RNA sequences can be any eukaryotic RNA polymerase II promoter.

In an aspect, a disclosed isolated nucleic acid molecule can restore one or more aspects of cellular homeostasis and/or cellular functionality and/or metabolic dysregulation. In an aspect, a disclosed isolated nucleic acid molecule can restore the functionality and/or structural integrity of a missing, deficient, and/or mutant protein or enzyme.

Disclosed herein is an expression cassette, an isolated nucleic acid molecule, comprising a nucleic acid sequence encoding a catalytically inactive PspdCas13b, a promoter operably linked to the nucleic acid sequence encoding the catalytically inactive PspdCas13b; and a polyadenylation signal.

Disclosed herein is an isolated nucleic acid molecule, comprising a nucleic acid sequence encoding a Cas13 alternative, a promoter operably linked to the nucleic acid sequence encoding the Cas13 alternative; and a polyadenylation signal. Disclosed herein is an expression cassette, an isolated nucleic acid molecule, comprising a nucleic acid sequence encoding a Cas13 alternative, a promoter operably linked to the nucleic acid sequence encoding the Cas13 alternative; and a polyadenylation signal. In an aspect, a Cas13 alternative can comprise alternatives known to the art including, but not limited to CRISPR-Inspired RNA Targeting System (CIRTS), Pumillo and FBF (PUF), rCas9, and/or any bifunctional RNA binding protein that can both bind to the trans-splicing RNA molecule and the target RNA to facilitate interaction between the two RNA species. RNA binding proteins are discussed in depth supra.

b. Internal Replacement Constructs

Disclosed herein is an isolated nucleic acid molecule comprising a nucleic acid sequence to be trans-spliced to a target endogenous pre-mRNA; a 3′ hemi intron; a 5′ hemi intron; two or more guide RNA sequences; a promoter operably linked to the two or more guide RNA sequences; and a nucleic acid sequence encoding one or more RNA binding proteins.

In an aspect, a disclosed isolated nucleic acid molecule can restore one or more aspects of cellular homeostasis and/or cellular functionality and/or metabolic dysregulation. In an aspect, a disclosed isolated nucleic acid molecule can restore the functionality and/or structural integrity of a missing, deficient, and/or mutant protein or enzyme.

In an aspect, a disclosed isolated nucleic acid molecule can further comprise a polyadenylation signal.

In an aspect, a disclosed isolated nucleic acid molecule can further comprise a spacer region. In an aspect, a disclosed spacer region can separate the 3′ splice region from the one or more guide RNA sequences. In an aspect, a disclosed spacer region can separate the 5′ splice region from the one or more guide RNA sequences. In an aspect, a disclosed spacer region can comprise any known spacer. In an aspect, a disclosed spacer region can comprise a consensus splicing motif (e.g., such as U1 or U2). In an aspect, a disclosed spacer region can comprise a limited number of consensus splicing motifs (e.g., such as U1 or U2).

In an aspect, a disclosed isolated nucleic acid molecule can further comprise two or more stem loops. In an aspect, a disclosed stem loop can be a cognate aptamer for a disclosed RNA binding protein. For example, in an aspect, a disclosed stem loop can be a direct repeat of the guide RNA scaffold for a disclosed Cas13d. In an aspect, a disclosed stem loop can facilitate interaction between a disclosed RNA molecule and a disclosed Cas protein.

In an aspect, a disclosed isolated nucleic acid molecule can further comprise a nuclear localization signal. In an aspect, a disclosed NLS can be comprise the sequence set forth in SEQ ID NO:60. In an aspect, a disclosed NLS can comprise any NLS known to the art. As known to the art (see, e.g., Lu J, et al. (2021) Cell Commun Signal. 19:60, which is incorporated herein by reference for its teachings of NLS), nuclear localization signals (NLS) are generally short peptides that act as a signal fragment that mediates the transport of proteins from the cytoplasm into the nucleus.

In an aspect, the two or more disclosed guide RNA sequences can directed the intron immediately 3′ to the target exon of the target endogenous pre-mRNA and the intron immediately 5′ to the target exon of the target endogenous pre-mRNA.

In an aspect, a disclosed 3′ hemi intron can comprise a branch point sequence, a polypyrimidine tract, and a 3′ splice acceptor site. In an aspect, a disclosed branch point sequence can comprise the sequence set forth in SEQ ID NO:57. In an aspect, a disclosed branch point sequence can be any eukaryotic branch point sequence known to the art. In an aspect, a disclosed 3′ splice acceptor site can comprise the sequence set forth in SEQ ID NO:58.

In an aspect of a disclosed isolated nucleic acid molecule, a disclosed 5′ hemi intron can comprise a consensus 5′ splice site. In an aspect, a disclosed 5′ splice site can comprise the sequence set forth in SEQ ID NO:59. In an aspect, a disclosed consensus 5′ splice site can comprise the sequence set forth in SEQ ID NO:61. In an aspect, a disclosed consequence 5′ splice site can comprise MAG|GURAGU (SEQ ID NO:61), wherein I denotes the exon intron junction, wherein M=A or C, and wherein R=A or G.

In an aspect, a disclosed 3′ hemi intron and/or a disclosed 5′ hemi intron can be recognized by nuclear splicing components within a host cell. In an aspect, a disclosed nucleic acid sequence encoding the RNA binding protein can interact with the two or more stem loops and/or can stabilize the two or more guide RNA sequences.

As known to the skilled person, RNA binding proteins (RBPs) can be important effectors of gene expression. RBPs can recognize hundreds of transcripts and form extensive regulatory networks that help to maintain cell homeostasis. Accordingly, the malfunction of RBPs underlies the origin of many diseases. In an aspect, a disclosed RNA binding protein can be any RNA binding protein having bispecific affinity for the trans-splicing RNA and the target pre-mRNA of interest. In an aspect, this affinity can be mediated by ribonucleoprotein interactions by, for example, Type VI CRISPR enzymes, or through direct RNA protein interactions by, for example, Pumillo and FBF (PUF) proteins. In an aspect, these interactions can be mediated by protein/aptamer interactions. RNA binding proteins are discussed in depth supra.

In an aspect, a disclosed RNA binding protein can comprise bispecific affinity for a disclosed target pre-mRNA as well as a disclosed Cas13 or a disclosed catalytically inactive Cas13. In an aspect, a disclosed Cas13 can comprise any catalytically inactive Cas13. For example, in an aspect, a disclosed Cas13 can comprise a catalytically inactive RfxCas13d or a catalytically inactive PspdCas13b. For example, in an aspect, a Cas13 alternative can comprise alternatives known to the art including, but not limited to CRISPR-Inspired RNA Targeting System (CIRTS), Pumillo and FBF (PUF), rCas9, and/or any bifunctional RNA binding protein that can both bind to the trans-splicing RNA molecule and the target RNA to facilitate interaction between the two RNA species. RNA binding proteins are discussed in depth supra.

In an aspect, a disclosed nucleic acid sequence to be trans-spliced can encode a translatable protein or a portion thereof. In an aspect, a disclosed portion can comprise one or more exons comprising a mutation. In an aspect, a disclosed portion can comprise some part of the gene sequence but not the complete sequence. For example, in an aspect, a disclosed portion can comprise the nucleic acid sequence having one or more mutations.

In an aspect, a disclosed nucleic acid sequence to be trans-spliced can encode DP71 or a portion thereof. DP71 is known to the art and discussed supra.

In an aspect, a disclosed nucleic acid sequence to be trans-spliced can encode DMPK or a portion thereof. DMPK is known to the art and discussed supra. In an aspect, the one or more disclosed guide RNA sequences can comprise the sequence set forth in SEQ ID NO:29 or SEQ ID NO:30 or a fragment thereof.

In an aspect, a disclosed nucleic acid sequence to be trans-spliced can encode DMD or a portion thereof. DMD is known to the art and discussed supra. In an aspect, the one or more disclosed guide RNA sequences can comprise the sequence set forth in SEQ ID NO:26, SEQ ID NO:27, or SEQ ID NO:28 or a fragment thereof.

In an aspect, a disclosed nucleic acid sequence to be trans-spliced can encode LMNA/C or a portion thereof. LMNA/C is known to the art and discussed supra. In an aspect, the one or more disclosed guide RNA sequences can comprise the sequence set forth in SEQ ID NO:31, SEQ ID NO:32, or SEQ ID NO:32 or a fragment thereof.

In an aspect, a disclosed nucleic acid sequence to be trans-spliced can encode CFTR or a portion thereof. CFTR is known to the art and discussed supra. In an aspect, a disclosed nucleic acid sequence to be trans-spliced can encode LRRK2 or a portion thereof. LRRK2 is known to the art and discussed supra.

In an aspect, a disclosed nucleic acid sequence to be trans-spliced can encode the protein or a portion thereof (such as, for example, Exon 1 or Exon 4, etc.) associated with the following genes: ABCA1, ABCA12, ABCA13, ABCA2, ABCA3, ABCA4, ABCA5, ABCC1, ABCC2, ABCC6, ABCC8, ABCC9, ACAN, ADAMTS13, ADCY10, ADGRV1, AGL, AGRN, AHDC1, ALK, ALMS1, ALPK3, ALS2, ANAPC1, ANK1, ANK2, ANK3, ANKRD11, ANKRD26, APC, APC2, APOB, ARFGEF2, ARHGAP31, ARHGEF10, ARHGEF18, ARID1A, ARIDIB, ARID2, ASH1L, ASPM, ASXL1, ASXL2, ASXL3, ATM, ATP7A, ATP7B, ATR, ATRX, BAZ1A, BAZ2B, BCOR, BCORL1, BDP1, BLM, BPTF, BRCA1, BRCA2, BRD4, BRWD3, C2CD3, C3, C5, CACNA1A, CACNA1B, CACNA1C, CACNA1D, CACNA1E, CACNA1F, CACNA1G, CACNA1H, CACNA1S, CAD, CAMTA1, CARMIL2, CC2D2A, CCDC88A, CCDC88C, CCNB3, CDH23, CDK13, CDK5RAP2, CELSR1, CEMIP2, CENPE, CENPF, CENPJ, CEP152, CEP164, CEP250, CEP290, CFAP43, CFAP44, CFAP65, CFTR/ABCC7, CHD1, CHD2, CHD3, CHD4, CHD7, CHD8, CIC, CIT, CLIP1, CLTC, CNOT1, CNTNAP1, COL11A1, COL11A2, COL12A1, COL17A1, COL18A1, COL1A1, COL1A2, COL27A1, COL2A1, COL3A1, COL4A1, COL4A2, COL4A3, COL4A4, COL4A5, COL4A6, COL5A1, COL5A2, COL6A3, COL7A1, CPAMD8, CPLANEl, CPS1, CPSF1, CRB1, CREBBP, CUBN, CUL7, CUX1, DCC, DCHS1, DEPDC5, DICERI, DIP2B, DLC1, DMD, DMXL2, DNAH1, DNAH11, DNAH17, DNAH2, DNAH5, DNAH7, DNAH8, DNAH9, DNMBP, DNMT1, DOCK2, DOCK3, DOCK6, DOCK7, DOCK8, DSCAM, DSP, DST, DUOX2, DYNCIHI, DYNC2H1, DYSF, EIF2AK4, EP300, EPG5, ERCC6, ERCC6L2, EXPH5, EYS, F5, F8, FANCA, FANCD2, FANCM, FAT1, FAT4, FBN1, FBN2, FLG, FLG2, FLNA, FLNB, FLNC, FLT4, FMN2, FN1, FRAS1, FREM1, FREM2, FSIP2, FYCO1, GLI2, GLI3, GPR179, GREBIL, GRIN2A, GRIN2B, GRIN2D, HCFC1, HECW2, HERC1, HERC2, HFM1, HIVEP1, HIVEP2, HMCN1, HSPG2, HTT, HUWE1, HYDIN, IFT140, IFT172, IGF1R, IGF2R, IGSF1, INSR, INTS1, IQSEC2, ITGB4, ITPR1, ITPR2, JMJD1C, KALRN, KANK1, KAT6A, KAT6B, KDM3B, KDM5B, KDM5C, KDM6A, KDM6B, KDR, KIAA0586, KIAA1109, KIAA1549, KIDINS220, KIF14, KIF1A, KIF1B, KIF21A, KIF26B, KIF7, KMT2A, KMT2B, KMT2C, KMT2D, KMT2E, KNL1, LAMA1, LAMA2, LAMA3, LAMA4, LAMA5, LAMB1, LAMB2, LAMC3, LCT, LOXHD1, LPA, LRBA, LRP1, LRP2, LRP4, LRP5, LRP6, LRPPRC, LRRK1, LRRK2, LTBP2, LTBP4, LYST, MACF1, MADD, MAGI2, MAP1B, MAP3K1, MAPK8IP3, MAPKBP1, MAST1, MBD5, MCM3AP, MED12, MED12L, MED13, MED13L, MED23, MEGF8, MET, MLH3, MPDZ, MSH6, MTOR, MYH10, MYH11, MYH14, MYH2, MYH3, MYH6, MYH7, MYH7B, MYH8, MYH9, MYLK, MYO15A, MYO18B, MYO3A, MYO5A, MYO5B, MYO7A, MYO9A, NALCN, NBAS, NBEA, NBEAL2, NCAPD2, NCAPD3, NEB, NEXMIF, NEXMIF, NF1, NFASC, NHS, NIN, NIPBL, NLRP1, NOTCH1, NOTCH2, NOTCH3, NPHP4, NRXN1, NRXN3, NSD1, NSD2, NUP155, NUP188, NUP205, OBSCN, OBSL1, OTOF, OTOG, OTOGL, PARD3, PBRM1, PCDH15, PCLO, PCNT, PHIP, PI4KA, PIEZO1, PIEZO2, PIK3C2A, PIKFYVE, PKD1, PKD1L1, PKHD1, PLCE1, PLEC, PLEKHG2, PNPLA6, POGZ, POLA1, POLE, POLR1A, POLR2A, POLR3A, PRG4, PRKDC, PRPF8, PRR12, PRX, PTCH1, PTPN23, PTPRF, PTPRJ, PTPRQ, PXDN, QRICH2, RAB3GAP2, RAIl, RALGAPA1, RANBP2, RB1CC1, RELN, RERE, REV3L, RIC1, RIMS1, RIMS2, RNF213, ROBO1, ROBO2, ROBO3, ROS1, RP1, RP1L1, RTTN, RUSC2, RYR1, RYR2, SACS, SAMD9, SAMD9L, SBF2, SCAPER, SCN10A, SCN11A, SCN1A, SCN2A, SCN3A, SCN4A, SCN5A, SCN8A, SCN9A, SETBP1, SETD1A, SETD1B, SETD2, SETD5, SETX, SHANK2, SHANK3, SHROOM4, SI, SIPA1L3, SLIT2, SLX4, SMARCA2, SMARCA4, SMCHD1, SNRNP200, SON, SPEF2, SPEG, SPG11, SPTA1, SPTAN1, SPTB, SPTBN2, SPTBN4, SRCAP, STRC, SVIL, SYNE1, SYNGAP1, SYNJ1, SZT2, TAF1, TANC2, TCF20, TCOF1, TDRD9, TECPR2, TECTA, TENM3, TENM4, TET3, TEX14, TEX15, TG, THOC2, TMEM94, TNC, TNIK, TNR, TNRC6B, TNXB, TOGARAMI, TONSL, TRIO, TRIOBP, TRIP11, TRIP12, TRPM1, TRPM6, TRPM7, TRRAP, TSC2, TTC37, TTN, TUBGCP6, UBR1, UNC80, USH2A, USP9X, VCAN, VPS13A, VPS13B, VPS13C, VPS13D, VWF, WDFY3, WDR19, WDR62, WDR81, WNK1, WRN, ZFHX2, ZFYVE26, ZNF142, ZNF292, ZNF335, ZNF407, ZNF462, ZNF469, or a portion thereof.

In an aspect, a disclosed nucleic acid sequence to be trans-spliced can encode a gene or a portion thereof (e.g., a specific exon such as an exon having a mutation) with a gene product that is directly or indirectly linked to one or more genetic diseases. Such genes include but are not limited to the following: dystrophin including mini- and micro-dystrophins (DMD; titin (TTN); titin cap (TCAP) α-sarcoglycan (SGCA), β-sarcoglycan (SGCB), γ-sarcoglycan (SGCG) or 6-sarcoglycan (SGCD); alpha-1-antitrypsin (A1-AT); myosin heavy chain 6 (MYH6); myosin heavy chain 7 (MYH7); myosin heavy chain 11 (MYH11); myosin light chain 2 (ML2); myosin light chain 3 (ML3); myosin light chain kinase 2 (MYLK2); myosin binding protein C (MYBPC3); desmin (DES); dynamin 2 (DNM2); laminin α2 (LAMA2); lamin A/C (LMNA); lamin B (LMNB); lamin B receptor (LBR); dysferlin (DYSF); emerin (EMD); insulin; blood clotting factors, including but not limited to, factor VIII and factor IX; erythropoietin (EPO); lipoprotein lipase (LPL); sarcoplasmic reticulum Ca2++-ATPase (SERCA2A), S100 calcium binding protein A1 (S100A1); myotubularin (MTM); DM1 protein kinase (DMPK); glycogen phosphorylase L (PYGL); glycogen phosphorylase, muscle associated (PYGM); glycogen synthase 1 (GYS1); glycogen synthase 2 (GYS2); α-galactosidase A (GLA); α-N-acetylgalactosaminidase (NAGA); acid α-glucosidase (GAA), sphingomyelinase phosphodiesterase 1 (SMPD1); lysosomal acid lipase (LIPA); collagen type I α1 chain (COL1A1); collagen type I α2 chain (COL1A2); collagen type III α1 chain (COL3A1); collagen type V al chain (COL5A1); collagen type V α2 chain (COL5A2); collagen type VI α1 chain (COL6A1); collagen type VI α2 chain (COL6A2); collagen type VI 3 chain (COL6A3); procollagen-lysine 2-oxoglutarate 5-dioxygenase (PLOD1); lysosomal acid lipase (LIPA); frataxin (FXN); myostatin (MSTN); β-N-acetyl hexosaminidase A (HEXA); β-N-acetylhexosaminidase B (HEXB); β-glucocerebrosidase (GBA); adenosine monophosphate deaminase 1 (AMPD1); β-globin (HBB); iduronidase (IDUA); iduronate 2-sulfate (IDS); troponin 1 (TNN13); troponin T2 (TNNT2); troponin C (TNNC1); tropomyosin 1 (TPM1); tropomyosin 3 (TPM3); N-acetyl-α-glucosaminidase (NAGLU); N-sulfoglucosamine sulfohydrolase (SGSH); heparan-α-glucosaminide N-acetyltransferase (HGSNAT); integrin a 7 (IGTA7); integrin a 9 (IGTA9); glucosamine(N-acetyl)-6-sulfatase (GNS); galactosamine(N-acetyl)-6-sulfatase (GALNS); (3-galactosidase (GLB1); β-glucuronidase (GUSB); hyaluronoglucosaminidase 1 (HYAL1); acid ceramidase (ASAHI); galactosylcermidase (GALC); cathepsin A (CTSA); cathepsin D (CTSA); cathepsin K (CTSK); GM2 ganglioside activator (GM2A); arylsulfatase A (ARSA); arylsulfatase B (ARSB); formylglycine-generating enzyme (SUMFI); neuraminidase 1 (NEU1); N-acetylglucosamine-1-phosphate transferase a (GNPTA); N-acetylglucosamine-1-phosphate transferase β (GNPTB); N-acetylglucosamine-1-phosphate transferase γ (GNPTG); mucolipin-1 (MCOLN1); NPC intracellular transporter 1 (NPC1); NPC intracellular transporter 2 (NPC2); ceroid lipofuscinosis 5 (CLN5); ceroid lipofuscinosis 6 (CLN6); ceroid lipofuscinosis 8 (CLN8); palmitoyl protein thioesterase 1 (PPT1); tripeptidyl peptidase 1 (TPP1); battenin (CLN3); DNAJ heat shock protein family 40 member C5 (DNAJC5); major facilitator superfamily domain containing 8 (MFSD8); mannosidase a class 2B member 1 (MAN2B1); mannosidase R (MANBA); aspartylglucosaminidase (AGA); α-L-fucosidase (FUCA1); cystinosin, lysosomal cysteine transporter (CTNS); sialin; solute carrier family 2 member 10 (SLC2A10); solute carrier family 17 member 5 (SLC17A5); solute carrier family 6 member 19 (SLC6A19); solute carrier family 22 member 5 (SLC22A5); solute carrier family 37 member 4 (SLC37A4); lysosomal associated membrane protein 2 (LAMP2); sodium voltage-gated channel α subunit 4 (SCN4A); sodium voltage-gated channel R subunit 4 (SCN4B); sodium voltage-gated channel α subunit 5 (SCN5A); sodium voltage-gated channel α subunit 4 (SCN4A); calcium voltage-gated channel subunit α1c (CACNA1C); calcium voltage-gated channel subunit α1s (CACNA1S); phosphoglycerate kinase 1 (PGK1); phosphoglycerate mutase 2 (PGAM2); amylo-α-1,6-glucosidase,4-α-glucanotransferase (AGL); potassium voltage-gated channel ISK-related subfamily member 1 (KCNE1); potassium voltage-gated channel ISK-related subfamily member 2 (KCNE2); potassium voltage-gated channel subfamily J member 2 (KCNJ2); potassium voltage-gated channel subfamily J member 5 (KCNJ5); potassium voltage-gated channel subfamily H member 2 (KCNH2); potassium voltage-gated channel KQT-like subfamily member 1 (KCNQ1); hyperpolarization-activated cyclic nucleotide-gated potassium channel 4 (HCN4); chloride voltage-gated channel 1 (CLCN1); camitine palmitoyltransferase 1A (CPT1A); ryanodine receptor 1 (RYR1); ryanodine receptor 2 (RYR2); bridging integrator 1 (BIN1); LARGE xylosyl- and glucuronyltransferase 1 (LARGEl); docking protein 7 (DOK7); fukutin (FKTN); fukutin related protein (FKRP); selenoprotein N (SELENON); protein O-mannosyltransferase 1 (POMT1); protein O-mannosyltransferase 2 (POMT2); protein O-linked mannose N-acetylglucosaminyltransferase 1 (POMGNT1); protein O-linked mannose N-acetylglucosaminyltransferase 2 (POMGNT2); protein-O-mannose kinase (POMK); isoprenoid synthase domain containing (ISPD); plectin (PLEC); cholinergic receptor nicotinic epsilon subunit (CHRNE); choline O-acetyltransferase (CHAT); choline kinase β (CHKB); collagen like tail subunit of asymmetric acetylcholinesterase (COLQ); receptor associated protein of the synapse (RAPSN); four and a half LIM domains 1 (FHL1); β-1,4-glucuronyltransferase 1 (B4GAT1); β-1,3-N-acetylgalactosaminyltransferase 2 (B3GALNT2); dystroglycan 1 (DAGI); transmembrane protein 5 (TMEM5); transmembrane protein 43 (TMEM43); SECIS binding protein 2 (SECISBP2); glucosamine (UDP-N-acetyl)-2-epimerase/N-acetylmannosamine kinase (GNE); anoctamin 5 (ANO5); structural maintenance of chromosomes flexible hinge domain containing 1 (SMCHD1); lactate dehydrogenase A (LDHA); lactate dehydrogenase B (LHDB); calpain 3 (CAPN3); caveolin 3 (CAV3); tripartite motif containing 32 (TRIM32); CCHC-type zinc finger nucleic acid binding protein (CNBP); nebulin (NEB); actin, α1, skeletal muscle (ACTA1); actin, α1, cardiac muscle (ACTC1); actinin α2 (ACTN2); poly(A)-binding protein nuclear 1 (PABPN1); LEM domain-containing protein 3 (LEMD3); zinc metalloproteinase STE24 (ZMPSTE24); microsomal triglyceride transfer protein (MTTP); cholinergic receptor nicotinic α1 subunit (CHRNA1); cholinergic receptor nicotinic α2 subunit (CHRNA2); cholinergic receptor nicotinic β3 subunit (CHRNA3); cholinergic receptor nicotinic β4 subunit (CHRNA4); cholinergic receptor nicotinic β5 subunit (CHRNA5); cholinergic receptor nicotinic α6 subunit (CHRNA6); cholinergic receptor nicotinic c7 subunit (CHRNA7); cholinergic receptor nicotinic α8 subunit (CHRNA8); cholinergic receptor nicotinic α9 subunit (CHRNA9); cholinergic receptor nicotinic α10 subunit (CHRNA10); cholinergic receptor nicotinic 31 subunit (CHRNB1); cholinergic receptor nicotinic β2 subunit (CHRNB2); cholinergic receptor nicotinic β3 subunit (CHRNB3); cholinergic receptor nicotinic β4 subunit (CHRNB4); cholinergic receptor nicotinic γ subunit (CHRNG1); cholinergic receptor nicotinic a subunit (CHRND); cholinergic receptor nicotinic E subunit (CHRNE1); ATP binding cassette subfamily A member 1 (ABCA1); ATP binding cassette subfamily C member 6 (ABCC6); ATP binding cassette subfamily C member 9 (ABCC9); ATP binding cassette subfamily D member 1 (ABCD1); ATPase sarcoplasmic/endoplasmic reticulum Ca2+ transporting 1 (ATP2A1); ATM serine/threonine kinase (ATM); a tocopherol transferase protein (TTPA); kinesin family member 21A (KIF21A); paired-like homeobox 2a (PHOX2A); heparan sulfate proteoglycan 2 (HSPG2); stromal interaction molecule 1 (STIM1); notch 1 (NOTCH1); notch 3 (NOTCH3); dystrobrevin a (DTNA); protein kinase AMP-activated, noncatalytic γ2 (PRKAG2); cysteine- and glycine-rich protein 3 (CSRP3); viniculin (VCL); myozenin 2 (MyoZ2); myopalladin (MYPN); junctophilin 2 (JPH2); phospholamban (PLN); calreticulin 3 (CALR3); nexilin F-actin-binding protein (NEXN); LIM domain binding 3 (LDB3); eyes absent 4 (EYA4); huntingtin (HTT); androgen receptor (AR); protein tyrosine phosphate non-receptor type 11 (PTPN11); junction plakoglobin (JUP); desmoplakin (DSP); plakophilin 2 (PKP2); desmoglein 2 (DSG2); desmocollin 2 (DSC2); catenin α3 (CTNNA3); NK2 homeobox 5 (NKX2-5); A-kinase anchor protein 9 (AKAP9); A-kinase anchor protein 10 (AKAP10); guanine nucleotide-binding protein α-inhibiting activity polypeptide 2 (GNAI2); ankyrin 2 (ANK2); syntrophin α-1 (SNTAT); calmodulin 1 (CALM1); calmodulin 2 (CALM2); HTRA serine peptidase 1 (HTRA1); fibrillin 1 (FBN1); fibrillin 2 (FBN2); xylosyltransferase 1 (XYLT1); xylosyltransferase 2 (XYLT2); tafazzin (TAZ); homogentisate 1,2-dioxygenase (HGD); glucose-6-phosphatase catalytic subunit (G6PC); 1,4-alpha-glucan enzyme 1 (GBE1); phosphofructokinase, muscle (PFKM); phosphorylase kinase regulatory subunit alpha 1 (PHKA1); phosphorylase kinase regulatory subunit alpha 2 (PHKA2); phosphorylase kinase regulatory subunit beta (PHKB); phosphorylase kinase catalytic subunit gamma 2 (PHKG2); phosphoglycerate mutase 2 (PGAM2); cystathionine-beta-synthase (CBS); methylenetetrahydrofolate reductase (MTHFR); 5-methyltetrahydrofolate-homocysteine methyltransferase (MTR); 5-methyltetrahydrofolate-homocysteine methyltransferase reductase (MTRR); methylmalonic aciduria and homocystinuria, cblD type (MMADHC); mitochondrial DNA, including, but not limited to mitochondrially encoded NADH:ubiquinone oxidoreductase core subunit 1 (MT-ND1); mitochondrially encoded NADH:ubiquinone oxidoreductase core subunit 5 (MT-ND5); mitochondrially encoded tRNA glutamic acid (MT-TE); mitochondrially encoded tRNA histadine (MT-TH); mitochondrially encoded tRNA leucine 1 (MT-TL1); mitochondrially encoded tRNA lysine (MT-TK); mitochondrially encoded tRNA serine 1 (MT-TS1); mitochondrially encoded tRNA valine (MT-TV); mitogen-activated protein kinase 1 (MAP2K1); B-Raf proto-oncogene, serine/threonine kinase (BRAF); raf-1 proto-oncogene, serine/threonine kinase (RAF1); growth factors, including, but not limited to insulin growth factor 1 (IGF-1); transforming growth factor β3 (TGFβ3); transforming growth factor β receptor, type I (TGFβ1); transforming growth factor β receptor, type II (TGFβ2), fibroblast growth factor 2 (FGF2), fibroblast growth factor 4 (FGF4), vascular endothelial growth factor A (VEGF-A), vascular endothelial growth factor B (VEGF-B); vascular endothelial growth factor C (VEGF-C), vascular endothelial growth factor D (VEGF-D), vascular endothelial growth factor receptor 1 (VEGFR1), and vascular endothelial growth factor receptor 2 (VEGFR2); interleukins; immunoadhesins; cytokines; and antibodies.

In an aspect, a disclosed nucleic acid sequence to be trans-spliced can be CpG depleted and codon-optimized for expression in a human cell. In an aspect, “CpG-free” can mean completely free of CpGs or partially free of CpGs. In an aspect, “CpG-free” can mean “CpG-depleted”. In an aspect, “CpG-depleted” can mean “CpG-free”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art.

In an aspect, a disclosed nucleic acid sequence to be trans-spliced can encode an RNA. In an aspect, a disclosed encoded RNA can comprise ribosomal RNA (rRNA), transfer RNA (tRNA), heterogeneous nuclear RNA (hnRNA), small nuclear RNA (snRNA), small nucleolar RNA (snoRNA), micro RNA (miRNA), Piwi-interacting RNA (piRNA), small interfering RNA (siRNA), short hairpin RNA (shRNA), singe guide RNA (sgRNA), non-coding RNA (ncRNA), long non-coding RNA (lncRNA), 7SL, Xist, short enhancer RNA (eRNA), circular RNA, intergenic RNA, or any combination thereof. In an aspect, a disclosed encoded RNA can comprise lncRNA, siRNA, shRNA, sgRNA, circular RNA, snoRNA, miRNA, or any combination thereof. In an aspect, a disclosed encoded RNA can comprise a functional non-coding RNA element.

In an aspect, a disclosed promoter for the two or more disclosed guide RNA sequences can be tissue-specific or ubiquitous and can be constitutive or inducible, depending on the pattern of the gene expression desired. A promoter can be native or foreign and can be a natural or a synthetic sequence. By foreign, it is intended that the transcriptional initiation region is not found in the wild-type host into which the transcriptional initiation region is introduced. In an aspect, a disclosed promoter can be a promoter/enhancer. In an aspect, a disclosed promoter for the two or more disclosed guide RNA sequences can be an endogenous promoter. In an aspect, a disclosed endogenous promoter can be an endogenous promoter/enhancer. In an aspect, a disclosed endogenous promoter or a disclosed endogenous promoter/enhancer can generally be obtained from a non-coding region upstream of a transcription initiation site of a gene of interest. In an aspect, a disclosed endogenous promoter or a disclosed endogenous promoter/enhancer can be used for constitutive and efficient expression of a disclosed gene. In an aspect, a disclosed promoter for the two or more disclosed guide RNA sequences can be a CMV promoter or a CMV promoter/enhancer. CMV promoters and CMV promoters/enhancers are well known to the art. In an aspect, a disclosed promoter for the one or more disclosed guide RNA sequences can be any eukaryotic RNA polymerase II promoter.

Disclosed herein is an expression cassette comprising an isolated nucleic acid molecule comprising a nucleic acid sequence to be trans-spliced to a target endogenous pre-mRNA; a 3′ hemi intron; a 5′ hemi intron; two or more guide RNA sequences; a promoter operably linked to the two or more guide RNA sequences; and a nucleic acid sequence encoding one or more RNA binding proteins.

Disclosed herein is an isolated nucleic acid molecule, comprising a nucleic acid sequence encoding a catalytically inactive RfxCas13d; a promoter operably linked to the nucleic acid sequence encoding the catalytically inactive RfxCas13d; and a polyadenylation signal.

In an aspect, a disclosed isolated nucleic acid molecule comprising a catalytically inactive RfxCas13d can further comprise a nuclear localization signal. In an aspect, a disclosed catalytically inactive RfxCas13d can comprise one or more inactivation mutations. In an aspect, a disclosed inactivation mutation can comprise R295A, H300A, R849A, H854A, or any combination thereof.

In an aspect, a disclosed Cas13d can further comprise one or more other agents or domains (e.g., is a fusion protein), such as one or more subcellular localization signals, one or more effector domains, or any combinations thereof.

In an aspect, a disclosed promoter for a catalytically inactive RfxCas13d can be tissue-specific or ubiquitous and can be constitutive or inducible, depending on the pattern of the gene expression desired. A promoter can be native or foreign and can be a natural or a synthetic sequence. By foreign, it is intended that the transcriptional initiation region is not found in the wild-type host into which the transcriptional initiation region is introduced. In an aspect, a disclosed promoter for a catalytically inactive RfxCas13d can be a promoter/enhancer. In an aspect, a disclosed promoter can be an endogenous promoter. In an aspect, a disclosed endogenous promoter can be an endogenous promoter/enhancer. In an aspect, a disclosed endogenous promoter or a disclosed endogenous promoter/enhancer can generally be obtained from a non-coding region upstream of a transcription initiation site of a gene of interest. In an aspect, a disclosed endogenous promoter or a disclosed endogenous promoter/enhancer can be used for constitutive and efficient expression of a disclosed gene. In an aspect, a disclosed promoter for a catalytically inactive RfxCas13d can be a CMV promoter or a CMV promoter/enhancer. CMV promoters and CMV promoters/enhancers are well known to the art. In an aspect, a disclosed promoter for the one or more disclosed guide RNA sequences can be any eukaryotic RNA polymerase II promoter.

In an aspect, a disclosed isolated nucleic acid molecule can restore one or more aspects of cellular homeostasis and/or cellular functionality and/or metabolic dysregulation. In an aspect, a disclosed isolated nucleic acid molecule can restore the functionality and/or structural integrity of a missing, deficient, and/or mutant protein or enzyme.

Disclosed herein is an expression cassette comprising an isolated nucleic acid molecule, comprising a nucleic acid sequence encoding a catalytically inactive RfxCas13d; a promoter operably linked to the nucleic acid sequence encoding the catalytically inactive RfxCas13d; and a polyadenylation signal.

Disclosed herein is an isolated nucleic acid molecule, comprising a nucleic acid sequence encoding a Cas13 alternative; a promoter operably linked to the nucleic acid sequence encoding the a Cas13 alternative; and a polyadenylation signal. Disclosed herein is an expression cassette comprising an isolated nucleic acid molecule, comprising a nucleic acid sequence encoding a Cas13 alternative; a promoter operably linked to the nucleic acid sequence encoding the Cas13 alternative; and a polyadenylation signal. In an aspect, a Cas13 alternative can comprise alternatives known to the art including, but not limited to CRISPR-Inspired RNA Targeting System (CIRTS), Pumillo and FBF (PUF), rCas9, and/or any bifunctional RNA binding protein that can both bind to the trans-splicing RNA molecule and the target RNA to facilitate interaction between the two RNA species. RNA binding proteins are discussed in depth supra.

### c. 3′ Replacement Constructs

Disclosed herein is an isolated nucleic acid molecule comprising a nucleic acid sequence to be trans-spliced to a target endogenous pre-mRNA; a 3′ hemi intron linked to the nucleic acid sequence to be trans-spliced; one or more guide RNA sequences; a promoter operably linked to the one or more guide RNA sequences; and a nucleic acid sequence encoding an RNA binding protein.

In an aspect, a disclosed isolated nucleic acid molecule can restore one or more aspects of cellular homeostasis and/or cellular functionality and/or metabolic dysregulation. In an aspect, a disclosed isolated nucleic acid molecule can restore the functionality and/or structural integrity of a missing, deficient, and/or mutant protein or enzyme.

In an aspect, a disclosed isolated nucleic acid molecule can further comprise a polyadenylation signal.

In an aspect, a disclosed isolated nucleic acid molecule can further comprise a spacer region. In an aspect, a disclosed spacer region can separate the 3′ splice region from the one or more guide RNA sequences. In an aspect, a disclosed spacer region can comprise any known spacer. In an aspect, a disclosed spacer region can comprise a consensus splicing motif (e.g., such as U1 or U2). In an aspect, a disclosed spacer region can comprise a limited number of consensus splicing motifs (e.g., such as U1 or U2).

In an aspect, a disclosed isolated nucleic acid molecule can further comprise one or more stem loops. In an aspect, a disclosed stem loop can be a cognate aptamer for a disclosed RNA binding protein. For example, in an aspect, a disclosed stem loop can be a direct repeat of the guide RNA scaffold for a disclosed Cas13d. In an aspect, a disclosed stem loop can facilitate interaction between a disclosed RNA molecule and a disclosed Cas protein.

In an aspect, a disclosed isolated nucleic acid molecule can further comprise a nuclear localization signal. In an aspect, a disclosed NLS can be comprise the sequence set forth in SEQ ID NO:60. In an aspect, a disclosed NLS can comprise any NLS known to the art. As known to the art (see, e.g., Lu J, et al. (2021) Cell Commun Signal. 19:60, which is incorporated herein by reference for its teachings of NLS), nuclear localization signals (NLS) are generally short peptides that act as a signal fragment that mediates the transport of proteins from the cytoplasm into the nucleus.

In an aspect, the one or more disclosed guide RNA sequences can direct the intron immediately 3′ to the last exon of the target endogenous pre-mRNA.

In an aspect, a disclosed 3′ hemi intron can comprise a branch point sequence, a polypyrimidine tract, and a 3′ splice acceptor site. In an aspect, a disclosed branch point sequence can comprise the sequence set forth in SEQ ID NO:57. In an aspect, a disclosed branch point sequence can be any eukaryotic branch point sequence known to the art. In an aspect, a disclosed 3′ splice acceptor site can comprise the sequence set forth in SEQ ID NO:58.

In an aspect, a disclosed 3′ hemi intron can be recognized by nuclear splicing components within a host cell. In an aspect, a disclosed nucleic acid sequence encoding the RNA binding protein can interact with the one or more stem loops and/or can stabilize the one or more guide RNA sequences.

As known to the skilled person, RNA binding proteins (RBPs) can be important effectors of gene expression. RBPs can recognize hundreds of transcripts and form extensive regulatory networks that help to maintain cell homeostasis. Accordingly, the malfunction of RBPs underlies the origin of many diseases. In an aspect, a disclosed RNA binding protein can be any RNA binding protein having bispecific affinity for the trans-splicing RNA and the target pre-mRNA of interest. In an aspect, this affinity can be mediated by riboncleoprotein interactions by, for example, Type VI CRISPR enzymes, or through direct RNA protein interactions by, for example, Pumillo and FBF (PUF) proteins. In an aspect, these interactions can be mediated by protein/aptamer interactions. RNA binding proteins are discussed in depth supra.

In an aspect, a disclosed RNA binding protein can comprise bispecific affinity for a disclosed target pre-mRNA as well as a disclosed Cas13 or a disclosed catalytically inactive Cas13. In an aspect, a disclosed Cas13 can comprise any catalytically inactive Cas13. For example, in an aspect, a disclosed Cas13 can comprise a catalytically inactive RfxCas13d or a catalytically inactive PspdCas13b. For example, in an aspect, a Cas13 alternative can comprise alternatives known to the art including, but not limited to CRISPR-Inspired RNA Targeting System (CIRTS), Pumillo and FBF (PUF), rCas9, and/or any bifunctional RNA binding protein that can both bind to the trans-splicing RNA molecule and the target RNA to facilitate interaction between the two RNA species. RNA binding proteins are discussed in depth supra.

In an aspect, a disclosed nucleic acid sequence to be trans-spliced can encode a translatable protein or a portion thereof. In an aspect, a disclosed portion can comprise one or more exons comprising a mutation. In an aspect, a disclosed portion can comprise some part of the gene sequence but not the complete sequence. For example, in an aspect, a disclosed portion can comprise the nucleic acid sequence having one or more mutations.

In an aspect, a disclosed nucleic acid sequence to be trans-spliced can encode DP71 or a portion thereof.

In an aspect, a disclosed nucleic acid sequence to be trans-spliced can encode DMPK or a portion thereof. In an aspect, the one or more disclosed guide RNA sequences can comprise the sequence set forth in SEQ ID NO:29 or SEQ ID NO:30 or a fragment thereof.

In an aspect, a disclosed nucleic acid sequence to be trans-spliced can encode DMD or a portion thereof. In an aspect, the one or more disclosed guide RNA sequences can comprise the sequence set forth in SEQ ID NO:26, SEQ ID NO:27, or SEQ ID NO:28 or a fragment thereof.

In an aspect, a disclosed nucleic acid sequence to be trans-spliced can encode LMNA/C or a portion thereof. In an aspect, the one or more disclosed guide RNA sequences can comprise the sequence set forth in SEQ ID NO:31, SEQ ID NO:32, or SEQ ID NO:32 or a fragment thereof.

In an aspect, a disclosed nucleic acid sequence to be trans-spliced can encode CFTR or a portion thereof. In an aspect, a disclosed nucleic acid sequence to be trans-spliced can encode LRRK2 or a portion thereof.

In an aspect, a disclosed nucleic acid sequence to be trans-spliced can encode the protein or a portion thereof (such as, for example, Exon 1 or Exon 4, etc.) associated with the following genes: ABCA1, ABCA12, ABCA13, ABCA2, ABCA3, ABCA4, ABCA5, ABCC1, ABCC2, ABCC6, ABCC8, ABCC9, ACAN, ADAMTS13, ADCY10, ADGRV1, AGL, AGRN, AHDC1, ALK, ALMS1, ALPK3, ALS2, ANAPC1, ANK1, ANK2, ANK3, ANKRD11, ANKRD26, APC, APC2, APOB, ARFGEF2, ARHGAP31, ARHGEF10, ARHGEF18, ARID1A, ARIDIB, ARID2, ASH1L, ASPM, ASXL1, ASXL2, ASXL3, ATM, ATP7A, ATP7B, ATR, ATRX, BAZ1A, BAZ2B, BCOR, BCORL1, BDP1, BLM, BPTF, BRCA1, BRCA2, BRD4, BRWD3, C2CD3, C3, C5, CACNA1A, CACNA1B, CACNA1C, CACNA1D, CACNA1E, CACNA1F, CACNA1G, CACNA1H, CACNA1S, CAD, CAMTA1, CARMIL2, CC2D2A, CCDC88A, CCDC88C, CCNB3, CDH23, CDK13, CDK5RAP2, CELSR1, CEMIP2, CENPE, CENPF, CENPJ, CEP152, CEP164, CEP250, CEP290, CFAP43, CFAP44, CFAP65, CFTR/ABCC7, CHD1, CHD2, CHD3, CHD4, CHD7, CHD8, CIC, CIT, CLIP1, CLTC, CNOT1, CNTNAP1, COL11A1, COL11A2, COL12A1, COL17A1, COL18A1, COL1A1, COL1A2, COL27A1, COL2A1, COL3A1, COL4A1, COL4A2, COL4A3, COL4A4, COL4A5, COL4A6, COL5A1, COL5A2, COL6A3, COL7A1, CPAMD8, CPLANEl, CPS1, CPSF1, CRB1, CREBBP, CUBN, CUL7, CUX1, DCC, DCHS1, DEPDC5, DICERI, DIP2B, DLC1, DMD, DMXL2, DNAH1, DNAH11, DNAH17, DNAH2, DNAH5, DNAH7, DNAH8, DNAH9, DNMBP, DNMT1, DOCK2, DOCK3, DOCK6, DOCK7, DOCK8, DSCAM, DSP, DST, DUOX2, DYNCIHI, DYNC2H1, DYSF, EIF2AK4, EP300, EPG5, ERCC6, ERCC6L2, EXPH5, EYS, F5, F8, FANCA, FANCD2, FANCM, FAT1, FAT4, FBN1, FBN2, FLG, FLG2, FLNA, FLNB, FLNC, FLT4, FMN2, FN1, FRAS1, FREM1, FREM2, FSIP2, FYCO1, GLI2, GLI3, GPR179, GREBIL, GRIN2A, GRIN2B, GRIN2D, HCFC1, HECW2, HERC1, HERC2, HFM1, HIVEP1, HIVEP2, HMCN1, HSPG2, HTT, HUWE1, HYDIN, IFT140, IFT172, IGF1R, IGF2R, IGSF1, INSR, INTS1, IQSEC2, ITGB4, ITPR1, ITPR2, JMJD1C, KALRN, KANK1, KAT6A, KAT6B, KDM3B, KDM5B, KDM5C, KDM6A, KDM6B, KDR, KIAA0586, KIAA1109, KIAA1549, KIDINS220, KIF14, KIF1A, KIF1B, KIF21A, KIF26B, KIF7, KMT2A, KMT2B, KMT2C, KMT2D, KMT2E, KNL1, LAMA1, LAMA2, LAMA3, LAMA4, LAMA5, LAMB1, LAMB2, LAMC3, LCT, LOXHD1, LPA, LRBA, LRP1, LRP2, LRP4, LRP5, LRP6, LRPPRC, LRRK1, LRRK2, LTBP2, LTBP4, LYST, MACF1, MADD, MAGI2, MAP1B, MAP3K1, MAPK8IP3, MAPKBP1, MAST1, MBD5, MCM3AP, MED12, MED12L, MED13, MED13L, MED23, MEGF8, MET, MLH3, MPDZ, MSH6, MTOR, MYH10, MYH11, MYH14, MYH2, MYH3, MYH6, MYH7, MYH7B, MYH8, MYH9, MYLK, MYO15A, MYO18B, MYO3A, MYO5A, MYO5B, MYO7A, MYO9A, NALCN, NBAS, NBEA, NBEAL2, NCAPD2, NCAPD3, NEB, NEXMIF, NEXMIF, NF1, NFASC, NHS, NIN, NIPBL, NLRP1, NOTCH1, NOTCH2, NOTCH3, NPHP4, NRXN1, NRXN3, NSD1, NSD2, NUP155, NUP188, NUP205, OBSCN, OBSL1, OTOF, OTOG, OTOGL, PARD3, PBRM1, PCDH15, PCLO, PCNT, PHIP, PI4KA, PIEZO1, PIEZO2, PIK3C2A, PIKFYVE, PKD1, PKD1L1, PKHD1, PLCE1, PLEC, PLEKHG2, PNPLA6, POGZ, POLA1, POLE, POLR1A, POLR2A, POLR3A, PRG4, PRKDC, PRPF8, PRR12, PRX, PTCH1, PTPN23, PTPRF, PTPRJ, PTPRQ, PXDN, QRICH2, RAB3GAP2, RAIL, RALGAPA1, RANBP2, RB1CC1, RELN, RERE, REV3L, RIC1, RIMS1, RIMS2, RNF213, ROBO1, ROBO2, ROBO3, ROS1, RP1, RP1L1, RTTN, RUSC2, RYR1, RYR2, SACS, SAMD9, SAMD9L, SBF2, SCAPER, SCN10A, SCN11A, SCN1A, SCN2A, SCN3A, SCN4A, SCN5A, SCN8A, SCN9A, SETBP1, SETD1A, SETD1B, SETD2, SETD5, SETX, SHANK2, SHANK3, SHROOM4, SI, SIPA1L3, SLIT2, SLX4, SMARCA2, SMARCA4, SMCHD1, SNRNP200, SON, SPEF2, SPEG, SPG11, SPTA1, SPTAN1, SPTB, SPTBN2, SPTBN4, SRCAP, STRC, SVIL, SYNE1, SYNGAP1, SYNJ1, SZT2, TAF1, TANC2, TCF20, TCOF1, TDRD9, TECPR2, TECTA, TENM3, TENM4, TET3, TEX14, TEX15, TG, THOC2, TMEM94, TNC, TNIK, TNR, TNRC6B, TNXB, TOGARAMI, TONSL, TRIO, TRIOBP, TRIP11, TRIP12, TRPM1, TRPM6, TRPM7, TRRAP, TSC2, TTC37, TTN, TUBGCP6, UBR1, UNC80, USH2A, USP9X, VCAN, VPS13A, VPS13B, VPS13C, VPS13D, VWF, WDFY3, WDR19, WDR62, WDR81, WNK1, WRN, ZFHX2, ZFYVE26, ZNF142, ZNF292, ZNF335, ZNF407, ZNF462, ZNF469, or a portion thereof.

In an aspect, a disclosed nucleic acid sequence to be trans-spliced can encode a gene or a portion thereof (e.g., a specific exon such as an exon having a mutation) with a gene product that is directly or indirectly linked to one or more genetic diseases. Such genes include but are not limited to the following: dystrophin including mini- and micro-dystrophins (DMD); titin (TTN); titin cap (TCAP) α-sarcoglycan (SGCA), β-sarcoglycan (SGCB), γ-sarcoglycan (SGCG) or 6-sarcoglycan (SGCD); alpha-1-antitrypsin (A1-AT); myosin heavy chain 6 (MYH6); myosin heavy chain 7 (MYH7); myosin heavy chain 11 (MYH11); myosin light chain 2 (ML2); myosin light chain 3 (ML3); myosin light chain kinase 2 (MYLK2); myosin binding protein C (MYBPC3); desmin (DES); dynamin 2 (DNM2); laminin α2 (LAMA2); lamin A/C (LMNA); lamin B (LMNB); lamin B receptor (LBR); dysferlin (DYSF); emerin (EMD); insulin; blood clotting factors, including but not limited to, factor VIII and factor IX; erythropoietin (EPO); lipoprotein lipase (LPL); sarcoplasmic reticulum Ca2++-ATPase (SERCA2A), S100 calcium binding protein A1 (S100A1); myotubularin (MTM); DM1 protein kinase (DMPK); glycogen phosphorylase L (PYGL); glycogen phosphorylase, muscle associated (PYGM); glycogen synthase 1 (GYS1); glycogen synthase 2 (GYS2); α-galactosidase A (GLA); α-N-acetylgalactosaminidase (NAGA); acid α-glucosidase (GAA), sphingomyelinase phosphodiesterase 1 (SMPD1); lysosomal acid lipase (LIPA); collagen type I α1 chain (COL1A1); collagen type I α2 chain (COL1A2); collagen type III α1 chain (COL3A1); collagen type V al chain (COL5A1); collagen type V α2 chain (COL5A2); collagen type VI α1 chain (COL6A1); collagen type VI α2 chain (COL6A2); collagen type VI 3 chain (COL6A3); procollagen-lysine 2-oxoglutarate 5-dioxygenase (PLOD1); lysosomal acid lipase (LIPA); frataxin (FXN); myostatin (MSTN); β-N-acetyl hexosaminidase A (HEXA); β-N-acetylhexosaminidase B (HEXB); β-glucocerebrosidase (GBA); adenosine monophosphate deaminase 1 (AMPD1); β-globin (HBB); iduronidase (IDUA); iduronate 2-sulfate (IDS); troponin 1 (TNN13); troponin T2 (TNNT2); troponin C (TNNC1); tropomyosin 1 (TPM1); tropomyosin 3 (TPM3); N-acetyl-α-glucosaminidase (NAGLU); N-sulfoglucosamine sulfohydrolase (SGSH); heparan-α-glucosaminide N-acetyltransferase (HGSNAT); integrin a 7 (IGTA7); integrin a 9 (IGTA9); glucosamine(N-acetyl)-6-sulfatase (GNS); galactosamine(N-acetyl)-6-sulfatase (GALNS); β-galactosidase (GLB1); β-glucuronidase (GUSB); hyaluronoglucosaminidase 1 (HYAL1); acid ceramidase (ASAHI); galactosylcermidase (GALC); cathepsin A (CTSA); cathepsin D (CTSA); cathepsin K (CTSK); GM2 ganglioside activator (GM2A); arylsulfatase A (ARSA); arylsulfatase B (ARSB); formylglycine-generating enzyme (SUMFI); neuraminidase 1 (NEU1); N-acetylglucosamine-1-phosphate transferase a (GNPTA); N-acetylglucosamine-1-phosphate transferase β (GNPTB); N-acetylglucosamine-1-phosphate transferase γ (GNPTG); mucolipin-1 (MCOLN1); NPC intracellular transporter 1 (NPC1); NPC intracellular transporter 2 (NPC2); ceroid lipofuscinosis 5 (CLN5); ceroid lipofuscinosis 6 (CLN6); ceroid lipofuscinosis 8 (CLN8); palmitoyl protein thioesterase 1 (PPT1); tripeptidyl peptidase 1 (TPP1); battenin (CLN3); DNAJ heat shock protein family 40 member C5 (DNAJC5); major facilitator superfamily domain containing 8 (MFSD8); mannosidase a class 2B member 1 (MAN2B1); mannosidase R (MANBA); aspartylglucosaminidase (AGA); α-L-fucosidase (FUCA1); cystinosin, lysosomal cysteine transporter (CTNS); sialin; solute carrier family 2 member 10 (SLC2A10); solute carrier family 17 member 5 (SLC17A5); solute carrier family 6 member 19 (SLC6A19); solute carrier family 22 member 5 (SLC22A5); solute carrier family 37 member 4 (SLC37A4); lysosomal associated membrane protein 2 (LAMP2); sodium voltage-gated channel α subunit 4 (SCN4A); sodium voltage-gated channel R subunit 4 (SCN4B); sodium voltage-gated channel α subunit 5 (SCN5A); sodium voltage-gated channel α subunit 4 (SCN4A); calcium voltage-gated channel subunit α1c (CACNA1C); calcium voltage-gated channel subunit α1s (CACNA1S); phosphoglycerate kinase 1 (PGK1); phosphoglycerate mutase 2 (PGAM2); amylo-α-1,6-glucosidase,4-α-glucanotransferase (AGL); potassium voltage-gated channel ISK-related subfamily member 1 (KCNE1); potassium voltage-gated channel ISK-related subfamily member 2 (KCNE2); potassium voltage-gated channel subfamily J member 2 (KCNJ2); potassium voltage-gated channel subfamily J member 5 (KCNJ5); potassium voltage-gated channel subfamily H member 2 (KCNH2); potassium voltage-gated channel KQT-like subfamily member 1 (KCNQ1); hyperpolarization-activated cyclic nucleotide-gated potassium channel 4 (HCN4); chloride voltage-gated channel 1 (CLCN1); camitine palmitoyltransferase 1A (CPT1A); ryanodine receptor 1 (RYR1); ryanodine receptor 2 (RYR2); bridging integrator 1 (BIN1); LARGE xylosyl- and glucuronyltransferase 1 (LARGEl); docking protein 7 (DOK7); fukutin (FKTN); fukutin related protein (FKRP); selenoprotein N (SELENON); protein O-mannosyltransferase 1 (POMT1); protein O-mannosyltransferase 2 (POMT2); protein O-linked mannose N-acetylglucosaminyltransferase 1 (POMGNT1); protein O-linked mannose N-acetylglucosaminyltransferase 2 (POMGNT2); protein-O-mannose kinase (POMK); isoprenoid synthase domain containing (ISPD); plectin (PLEC); cholinergic receptor nicotinic epsilon subunit (CHRNE); choline O-acetyltransferase (CHAT); choline kinase β (CHKB); collagen like tail subunit of asymmetric acetylcholinesterase (COLQ); receptor associated protein of the synapse (RAPSN); four and a half LIM domains 1 (FHL1); β-1,4-glucuronyltransferase 1 (B4GAT1); β-1,3-N-acetylgalactosaminyltransferase 2 (B3GALNT2); dystroglycan 1 (DAGI); transmembrane protein 5 (TMEM5); transmembrane protein 43 (TMEM43); SECIS binding protein 2 (SECISBP2); glucosamine (UDP-N-acetyl)-2-epimerase/N-acetylmannosamine kinase (GNE); anoctamin 5 (ANO5); structural maintenance of chromosomes flexible hinge domain containing 1 (SMCHD1); lactate dehydrogenase A (LDHA); lactate dehydrogenase B (LHDB); calpain 3 (CAPN3); caveolin 3 (CAV3); tripartite motif containing 32 (TRIM32); CCHC-type zinc finger nucleic acid binding protein (CNBP); nebulin (NEB); actin, α1, skeletal muscle (ACTA1); actin, α1, cardiac muscle (ACTC1); actinin α2 (ACTN2); poly(A)-binding protein nuclear 1 (PABPN1); LEM domain-containing protein 3 (LEMD3); zinc metalloproteinase STE24 (ZMPSTE24); microsomal triglyceride transfer protein (MTTP); cholinergic receptor nicotinic α1 subunit (CHRNA1); cholinergic receptor nicotinic α2 subunit (CHRNA2); cholinergic receptor nicotinic α3 subunit (CHRNA3); cholinergic receptor nicotinic β4 subunit (CHRNA4); cholinergic receptor nicotinic β5 subunit (CHRNA5); cholinergic receptor nicotinic α6 subunit (CHRNA6); cholinergic receptor nicotinic α7 subunit (CHRNA7); cholinergic receptor nicotinic α8 subunit (CHRNA8); cholinergic receptor nicotinic α9 subunit (CHRNA9); cholinergic receptor nicotinic β10 subunit (CHRNA10); cholinergic receptor nicotinic 31 subunit (CHRNB1); cholinergic receptor nicotinic β2 subunit (CHRNB2); cholinergic receptor nicotinic β3 subunit (CHRNB3); cholinergic receptor nicotinic β4 subunit (CHRNB4); cholinergic receptor nicotinic γ subunit (CHRNG1); cholinergic receptor nicotinic a subunit (CHRND); cholinergic receptor nicotinic E subunit (CHRNE1); ATP binding cassette subfamily A member 1 (ABCA1); ATP binding cassette subfamily C member 6 (ABCC6); ATP binding cassette subfamily C member 9 (ABCC9); ATP binding cassette subfamily D member 1 (ABCD1); ATPase sarcoplasmic/endoplasmic reticulum Ca2+ transporting 1 (ATP2A1); ATM serine/threonine kinase (ATM); a tocopherol transferase protein (TTPA); kinesin family member 21A (KIF21A); paired-like homeobox 2a (PHOX2A); heparan sulfate proteoglycan 2 (HSPG2); stromal interaction molecule 1 (STIM1); notch 1 (NOTCH1); notch 3 (NOTCH3); dystrobrevin a (DTNA); protein kinase AMP-activated, noncatalytic γ2 (PRKAG2); cysteine- and glycine-rich protein 3 (CSRP3); viniculin (VCL); myozenin 2 (MyoZ2); myopalladin (MYPN); junctophilin 2 (JPH2); phospholamban (PLN); calreticulin 3 (CALR3); nexilin F-actin-binding protein (NEXN); LIM domain binding 3 (LDB3); eyes absent 4 (EYA4); huntingtin (HTT); androgen receptor (AR); protein tyrosine phosphate non-receptor type 11 (PTPN11); junction plakoglobin (JUP); desmoplakin (DSP); plakophilin 2 (PKP2); desmoglein 2 (DSG2); desmocollin 2 (DSC2); catenin α3 (CTNNA3); NK2 homeobox 5 (NKX2-5); A-kinase anchor protein 9 (AKAP9); A-kinase anchor protein 10 (AKAP10); guanine nucleotide-binding protein α-inhibiting activity polypeptide 2 (GNAI2); ankyrin 2 (ANK2); syntrophin α-1 (SNTAT); calmodulin 1 (CALM1); calmodulin 2 (CALM2); HTRA serine peptidase 1 (HTRA1); fibrillin 1 (FBN1); fibrillin 2 (FBN2); xylosyltransferase 1 (XYLT1); xylosyltransferase 2 (XYLT2); tafazzin (TAZ); homogentisate 1,2-dioxygenase (HGD); glucose-6-phosphatase catalytic subunit (G6PC); 1,4-alpha-glucan enzyme 1 (GBE1); phosphofructokinase, muscle (PFKM); phosphorylase kinase regulatory subunit alpha 1 (PHKA1); phosphorylase kinase regulatory subunit alpha 2 (PHKA2); phosphorylase kinase regulatory subunit beta (PHKB); phosphorylase kinase catalytic subunit gamma 2 (PHKG2); phosphoglycerate mutase 2 (PGAM2); cystathionine-beta-synthase (CBS); methylenetetrahydrofolate reductase (MTHFR); 5-methyltetrahydrofolate-homocysteine methyltransferase (MTR); 5-methyltetrahydrofolate-homocysteine methyltransferase reductase (MTRR); methylmalonic aciduria and homocystinuria, cblD type (MMADHC); mitochondrial DNA, including, but not limited to mitochondrially encoded NADH:ubiquinone oxidoreductase core subunit 1 (MT-ND1); mitochondrially encoded NADH:ubiquinone oxidoreductase core subunit 5 (MT-ND5); mitochondrially encoded tRNA glutamic acid (MT-TE); mitochondrially encoded tRNA histadine (MT-TH); mitochondrially encoded tRNA leucine 1 (MT-TL1); mitochondrially encoded tRNA lysine (MT-TK); mitochondrially encoded tRNA serine 1 (MT-TS1); mitochondrially encoded tRNA valine (MT-TV); mitogen-activated protein kinase 1 (MAP2K1); B-Raf proto-oncogene, serine/threonine kinase (BRAF); raf-1 proto-oncogene, serine/threonine kinase (RAF1); growth factors, including, but not limited to insulin growth factor 1 (IGF-1); transforming growth factor β3 (TGFβ3); transforming growth factor β receptor, type I (TGFβ1); transforming growth factor β receptor, type II (TGFβ2), fibroblast growth factor 2 (FGF2), fibroblast growth factor 4 (FGF4), vascular endothelial growth factor A (VEGF-A), vascular endothelial growth factor B (VEGF-B); vascular endothelial growth factor C (VEGF-C), vascular endothelial growth factor D (VEGF-D), vascular endothelial growth factor receptor 1 (VEGFR1), and vascular endothelial growth factor receptor 2 (VEGFR2); interleukins; immunoadhesins; cytokines; and antibodies.

In an aspect, a disclosed nucleic acid sequence to be trans-spliced can be CpG depleted and codon-optimized for expression in a human cell. In an aspect, “CpG-free” can mean completely free of CpGs or partially free of CpGs. In an aspect, “CpG-free” can mean “CpG-depleted”. In an aspect, “CpG-depleted” can mean “CpG-free”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art.

In an aspect, a disclosed nucleic acid sequence to be trans-spliced can encode an RNA. In an aspect, a disclosed encoded RNA can comprise ribosomal RNA (rRNA), transfer RNA (tRNA), heterogeneous nuclear RNA (hnRNA), small nuclear RNA (snRNA), small nucleolar RNA (snoRNA), micro RNA (miRNA), Piwi-interacting RNA (piRNA), small interfering RNA (siRNA), short hairpin RNA (shRNA), singe guide RNA (sgRNA), non-coding RNA (ncRNA), long non-coding RNA (lncRNA), 7SL, Xist, short enhancer RNA (eRNA), circular RNA, intergenic RNA, or any combination thereof. In an aspect, a disclosed encoded RNA can comprise lncRNA, siRNA, shRNA, sgRNA, circular RNA, snoRNA, miRNA, or any combination thereof. In an aspect, a disclosed encoded RNA can comprise a functional non-coding RNA element.

In an aspect, a disclosed promoter for the one or more disclosed guide RNA sequences can be tissue-specific or ubiquitous and can be constitutive or inducible, depending on the pattern of the gene expression desired. A promoter can be native or foreign and can be a natural or a synthetic sequence. By foreign, it is intended that the transcriptional initiation region is not found in the wild-type host into which the transcriptional initiation region is introduced. In an aspect, a disclosed promoter can be a promoter/enhancer. In an aspect, a disclosed promoter for the one or more disclosed guide RNA sequences can be an endogenous promoter. In an aspect, a disclosed endogenous promoter can be an endogenous promoter/enhancer. In an aspect, a disclosed endogenous promoter or a disclosed endogenous promoter/enhancer can generally be obtained from a non-coding region upstream of a transcription initiation site of a gene of interest. In an aspect, a disclosed endogenous promoter or a disclosed endogenous promoter/enhancer can be used for constitutive and efficient expression of a disclosed gene. In an aspect, a disclosed promoter for the one or more disclosed guide RNA sequences can be a CMV promoter or a CMV promoter/enhancer. CMV promoters and CMV promoters/enhancers are well known to the art. In an aspect, a disclosed promoter for the one or more disclosed guide RNA sequences can be any eukaryotic RNA polymerase II promoter.

Disclosed herein is an expression cassette comprising an isolated nucleic acid molecule comprising a nucleic acid sequence to be trans-spliced to a target endogenous pre-mRNA; a 3′ hemi intron linked to the nucleic acid sequence to be trans-spliced; one or more guide RNA sequences; a promoter operably linked to the one or more guide RNA sequences; and a nucleic acid sequence encoding an RNA binding protein.

Disclosed herein is an isolated nucleic acid molecule, comprising a nucleic acid sequence encoding a catalytically inactive RfxCas13d; a promoter operably linked to the nucleic acid sequence encoding the catalytically inactive RfxCas13d; and a polyadenylation signal.

In an aspect, a disclosed isolated nucleic acid molecule comprising a catalytically inactive RfxCas13d can further comprise a nuclear localization signal. In an aspect, a disclosed catalytically inactive RfxCas13d can comprise one or more inactivation mutations. In an aspect, a disclosed inactivation mutation can comprise R295A, H300A, R849A, H854A, or any combination thereof.

In an aspect, a disclosed Cas13d can further comprise one or more other agents or domains (e.g., is a fusion protein), such as one or more subcellular localization signals, one or more effector domains, or any combinations thereof.

In an aspect, a disclosed promoter for a catalytically inactive RfxCas13d can be tissue-specific or ubiquitous and can be constitutive or inducible, depending on the pattern of the gene expression desired. A promoter can be native or foreign and can be a natural or a synthetic sequence. By foreign, it is intended that the transcriptional initiation region is not found in the wild-type host into which the transcriptional initiation region is introduced. In an aspect, a disclosed promoter can be a promoter/enhancer. In an aspect, a disclosed promoter for a catalytically inactive RfxCas13d can be an endogenous promoter. In an aspect, a disclosed endogenous promoter can be an endogenous promoter/enhancer. In an aspect, a disclosed endogenous promoter or a disclosed endogenous promoter/enhancer can generally be obtained from a non-coding region upstream of a transcription initiation site of a gene of interest. In an aspect, a disclosed endogenous promoter or a disclosed endogenous promoter/enhancer can be used for constitutive and efficient expression of a disclosed gene. In an aspect, a disclosed promoter for a catalytically inactive RfxCas13d can be a CMV promoter or a CMV promoter/enhancer. CMV promoters and CMV promoters/enhancers are well known to the art. In an aspect, a disclosed promoter for the one or more disclosed guide RNA sequences can be any eukaryotic RNA polymerase II promoter.

In an aspect, a disclosed isolated nucleic acid molecule can restore one or more aspects of cellular homeostasis and/or cellular functionality and/or metabolic dysregulation. In an aspect, a disclosed isolated nucleic acid molecule can restore the functionality and/or structural integrity of a missing, deficient, and/or mutant protein or enzyme.

Disclosed herein is an expression cassette comprising an isolated nucleic acid molecule, comprising a nucleic acid sequence encoding a catalytically inactive RfxCas13d; a promoter operably linked to the nucleic acid sequence encoding the catalytically inactive RfxCas13d; and a polyadenylation signal.

Disclosed herein is an isolated nucleic acid molecule, comprising a nucleic acid sequence encoding a Cas13 alternative; a promoter operably linked to the nucleic acid sequence encoding the Cas13 alternative; and a polyadenylation signal. Disclosed herein is an expression cassette comprising an isolated nucleic acid molecule, comprising a nucleic acid sequence encoding a Cas13 alternative; a promoter operably linked to the nucleic acid sequence encoding the Cas13 alternative; and a polyadenylation signal. In an aspect, a Cas13 alternative can comprise alternatives known to the art including, but not limited to CRISPR-Inspired RNA Targeting System (CIRTS), Pumillo and FBF (PUF), rCas9, and/or any bifunctional RNA binding protein that can both bind to the trans-splicing RNA molecule and the target RNA to facilitate interaction between the two RNA species. RNA binding proteins are discussed in depth supra.

### 2. Transcriptome Engineering Systems

Disclosed herein is a transcriptome engineering system comprising (i) a first isolated nucleic acid molecule comprising a nucleic acid sequence to be trans-spliced to a target endogenous pre-mRNA; a 3′ hemi intron linked to the nucleic acid sequence to be trans-spliced; one or more guide RNA sequences; a promoter operably linked to the one or more guide RNA sequences; and a nucleic acid sequence encoding an RNA binding protein; and (ii) a second isolated nucleic acid molecule comprising a nucleic acid sequence encoding a catalytically inactive RfxCas13d, a promoter operably linked to the nucleic acid sequence encoding the catalytically inactive RfxCas13d; and a polyadenylation signal.

In an aspect, a disclosed first isolated nucleic acid molecule and a disclosed second isolated nucleic acid molecule can form a ternary complex with the endogenous pre-mRNA molecule. In an aspect, a disclosed resulting chimeric RNA molecule can comprise the trans-spliced nucleic acid sequence.

Disclosed herein is a transcriptome engineering system comprising (i) a first isolated nucleic acid molecule comprising a nucleic acid sequence to be trans-spliced to a target endogenous pre-mRNA, a 5′ hemi intron linked to the nucleic acid sequence to be trans-spliced; one or more guide RNA sequences; a promoter operably linked to the one or more guide RNA sequences; and a nucleic acid sequence encoding an RNA binding protein; and (ii) a second isolated nucleic acid molecule comprising a nucleic acid sequence encoding a catalytically inactive PspdCas13b, a promoter operably linked to the nucleic acid sequence encoding the catalytically inactive PspdCas13b; and a polyadenylation signal.

In an aspect, a disclosed first isolated nucleic acid molecule and a disclosed second isolated nucleic acid molecule can form a ternary complex with the endogenous pre-mRNA molecule. In an aspect, a disclosed resulting chimeric RNA molecule can comprise the trans-spliced nucleic acid sequence.

Disclosed herein is a transcriptome engineering system, comprising (i) a first isolated nucleic acid molecule comprising a nucleic acid sequence to be trans-spliced to a target endogenous pre-mRNA; a 3′ hemi intron; a 5′ hemi intron; two or more guide RNA sequences; a promoter operably linked to the two or more guide RNA sequences; and a nucleic acid sequence encoding one or more RNA binding proteins; and (ii) a second isolated nucleic acid molecule comprising a nucleic acid sequence encoding a catalytically inactive RfxCas13d, a promoter operably linked to the nucleic acid sequence encoding the catalytically inactive RfxCas13d; and a polyadenylation signal. In an aspect, a disclosed first isolated nucleic acid molecule and a disclosed second isolated nucleic acid molecule can form a ternary complex with the endogenous pre-mRNA molecule. In an aspect, a disclosed resulting chimeric RNA molecule can comprise the trans-spliced nucleic acid sequence.

Disclosed herein is a transcriptome engineering system comprising (i) a first isolated nucleic acid molecule comprising a nucleic acid sequence to be trans-spliced to a target endogenous pre-mRNA; a 3′ hemi intron linked to the nucleic acid sequence to be trans-spliced; one or more guide RNA sequences; a promoter operably linked to the one or more guide RNA sequences; and a nucleic acid sequence encoding an RNA binding protein; and (ii) a second isolated nucleic acid molecule comprising a nucleic acid sequence encoding a Cas13 alternative, a promoter operably linked to the nucleic acid sequence encoding the Cas13 alternative; and a polyadenylation signal. In an aspect, a disclosed first isolated nucleic acid molecule and a disclosed second isolated nucleic acid molecule can form a ternary complex with the endogenous pre-mRNA molecule. In an aspect, a disclosed resulting chimeric RNA molecule can comprise the trans-spliced nucleic acid sequence.

Disclosed herein is a transcriptome engineering system comprising (i) a first isolated nucleic acid molecule comprising a nucleic acid sequence to be trans-spliced to a target endogenous pre-mRNA, a 5′ hemi intron linked to the nucleic acid sequence to be trans-spliced; one or more guide RNA sequences; a promoter operably linked to the one or more guide RNA sequences; and a nucleic acid sequence encoding an RNA binding protein; and (ii) a second isolated nucleic acid molecule comprising a nucleic acid sequence encoding a Cas13 alternative, a promoter operably linked to the nucleic acid sequence encoding the Cas13 alternative; and a polyadenylation signal. In an aspect, a disclosed first isolated nucleic acid molecule and a disclosed second isolated nucleic acid molecule can form a ternary complex with the endogenous pre-mRNA molecule. In an aspect, a disclosed resulting chimeric RNA molecule can comprise the trans-spliced nucleic acid sequence.

Disclosed herein is a transcriptome engineering system, comprising (i) a first isolated nucleic acid molecule comprising a nucleic acid sequence to be trans-spliced to a target endogenous pre-mRNA; a 3′ hemi intron; a 5′ hemi intron; two or more guide RNA sequences; a promoter operably linked to the two or more guide RNA sequences; and a nucleic acid sequence encoding one or more RNA binding proteins; and (ii) a second isolated nucleic acid molecule comprising a nucleic acid sequence encoding a Cas13 alternative, a promoter operably linked to the nucleic acid sequence encoding the Cas13 alternative; and a polyadenylation signal. In an aspect, a disclosed first isolated nucleic acid molecule and a disclosed second isolated nucleic acid molecule can form a ternary complex with the endogenous pre-mRNA molecule. In an aspect, a disclosed resulting chimeric RNA molecule can comprise the trans-spliced nucleic acid sequence.

In an aspect, a Cas13 alternative can comprise alternatives known to the art including, but not limited to CRISPR-Inspired RNA Targeting System (CIRTS), Pumillo and FBF (PUF), rCas9, and/or any bifunctional RNA binding protein that can both bind to the trans-splicing RNA molecule and the target RNA to facilitate interaction between the two RNA species. RNA binding proteins are discussed in depth supra.

### 3. Vectors

Disclosed herein is a vector comprising a disclosed isolated nucleic acid molecule.

Disclosed herein is a vector comprising one or more disclosed isolated nucleic acid molecules.

Disclosed herein is a vector comprising an isolated nucleic acid molecule comprising a nucleic acid sequence to be trans-spliced to a target endogenous pre-mRNA; a 5′ hemi intron linked to the nucleic acid sequence to be trans-spliced; one or more guide RNA sequences; a promoter operably linked to the one or more guide RNA sequences; and a nucleic acid sequence encoding an RNA binding protein.

Disclosed herein is a vector comprising an isolated nucleic acid molecule comprising a nucleic acid sequence to be trans-spliced to a target endogenous pre-mRNA; a 3′ hemi intron; a 5′ hemi intron; two or more guide RNA sequences; a promoter operably linked to the two or more guide RNA sequences; and a nucleic acid sequence encoding one or more RNA binding proteins.

Disclosed herein is a vector comprising an isolated nucleic acid molecule comprising a nucleic acid sequence to be trans-spliced to a target endogenous pre-mRNA; a 3′ hemi intron linked to the nucleic acid sequence to be trans-spliced; one or more guide RNA sequences; a promoter operably linked to the one or more guide RNA sequences; and a nucleic acid sequence encoding an RNA binding protein.

Disclosed herein is a vector comprising an isolated nucleic acid molecule comprising a nucleic acid sequence encoding a catalytically inactive RfxCas13d; a promoter operably linked to the nucleic acid sequence encoding the catalytically inactive RfxCas13d; and a poly adenylation signal.

Disclosed herein is a vector comprising an isolated nucleic acid molecule comprising a nucleic acid sequence encoding a catalytically inactive PspdCas13b, a promoter operably linked to the nucleic acid sequence encoding the catalytically inactive PspdCas13b; and a polyadenylation signal.

Disclosed herein is a vector comprising an isolated nucleic acid molecule comprising a nucleic acid sequence encoding a Cas13 alternative; a promoter operably linked to the nucleic acid sequence encoding the Cas13 alternative; and a polyadenylation signal. In an aspect, a Cas13 alternative can comprise alternatives known to the art including, but not limited to CRISPR-Inspired RNA Targeting System (CIRTS), Pumillo and FBF (PUF), rCas9, and/or any bifunctional RNA binding protein that can both bind to the trans-splicing RNA molecule and the target RNA to facilitate interaction between the two RNA species. RNA binding proteins are discussed in depth supra.

Disclosed herein is a vector comprising an expression cassette comprising an isolated nucleic acid molecule comprising a nucleic acid sequence to be trans-spliced to a target endogenous pre-mRNA; a 5′ hemi intron linked to the nucleic acid sequence to be trans-spliced; one or more guide RNA sequences; a promoter operably linked to the one or more guide RNA sequences; and a nucleic acid sequence encoding an RNA binding protein.

Disclosed herein is a vector comprising an expression cassette comprising an isolated nucleic acid molecule comprising a nucleic acid sequence to be trans-spliced to a target endogenous pre-mRNA; a 3′ hemi intron linked to the nucleic acid sequence to be trans-spliced; one or more guide RNA sequences; a promoter operably linked to the one or more guide RNA sequences; and a nucleic acid sequence encoding an RNA binding protein.

Disclosed herein is a vector comprising an expression cassette comprising an isolated nucleic acid molecule comprising a nucleic acid sequence to be trans-spliced to a target endogenous pre-mRNA; a 3′ hemi intron; a 5′ hemi intron; two or more guide RNA sequences; a promoter operably linked to the two or more guide RNA sequences; and a nucleic acid sequence encoding one or more RNA binding proteins.

Disclosed herein is a vector comprising an expression cassette comprising an isolated nucleic acid molecule, comprising a nucleic acid sequence encoding a catalytically inactive RfxCas13d; a promoter operably linked to the nucleic acid sequence encoding the catalytically inactive RfxCas13d; and a polyadenylation signal.

Disclosed herein is a vector comprising an expression cassette an isolated nucleic acid molecule, comprising a nucleic acid sequence encoding a catalytically inactive PspdCas13b, a promoter operably linked to the nucleic acid sequence encoding the catalytically inactive PspdCas13b; and a polyadenylation signal.

Disclosed herein is a vector comprising an expression cassette an isolated nucleic acid molecule, comprising a nucleic acid sequence encoding a Cas13 alternative, a promoter operably linked to the nucleic acid sequence encoding the Cas13 alternative; and a polyadenylation signal. In an aspect, a Cas13 alternative can comprise alternatives known to the art including, but not limited to CRISPR-Inspired RNA Targeting System (CIRTS), Pumillo and FBF (PUF), rCas9, and/or any bifunctional RNA binding protein that can both bind to the trans-splicing RNA molecule and the target RNA to facilitate interaction between the two RNA species. RNA binding proteins are discussed in depth supra.

In an aspect, a disclosed vector can restore one or more aspects of cellular homeostasis and/or cellular functionality and/or metabolic dysregulation. In an aspect, a disclosed vector can restore the functionality and/or structural integrity of a missing, deficient, and/or mutant protein or enzyme.

In an aspect, a therapeutically effective amount of disclosed vector can be delivered via intravenous (IV) administration and can comprise a range of about 1×10¹⁰ vg/kg to about 2×10¹⁴ vg/kg. In an aspect, for example, a disclosed vector can be administered at a dose of about 1×10¹¹ to about 8×10¹³ vg/kg or about 1×10¹² to about 8×10¹³ vg/kg. In an aspect, a disclosed vector can be administered at a dose of about 1×10¹³ to about 6×10¹³ vg/kg. In an aspect, a disclosed vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results. In an aspect, a therapeutically effective amount of disclosed vector can comprise a range determined by a skilled person.

In an aspect, a disclosed nucleic acid sequence can have a coding sequence that is less than about 4.5 kilobases.

In an aspect, a disclosed vector can be a viral vector or a non-viral vector. In an aspect, a disclosed non-viral vector can be a polymer-based vector, a peptide-based vector, a lipid nanoparticle, a solid lipid nanoparticle, or a cationic lipid-based vector. In an aspect, a disclosed vector can comprise exosomes, extracellular vesicles, and virus like particles. In an aspect, a disclosed viral vector can be an adenovirus vector, an AAV vector, a herpes simplex virus vector, a retrovirus vector, a lentivirus vector, and alphavirus vector, a Flavivirus vector, a rhabdovirus vector, a measles virus vector, a Newcastle disease viral vector, a poxvirus vector, or a picornavirus vector.

In an aspect, a disclosed viral vector can be an adeno-associated virus (AAV) vector In an aspect, a disclosed AAV vector can include naturally isolated serotypes including, but not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7 as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In an aspect, an AAV capsid can be a chimera either created by capsid evolution or by rational capsid engineering from a naturally isolated AAV variants to capture desirable serotype features such as enhanced or specific tissue tropism and/or a host immune response escape. Naturally isolated AAV variants include, but not limited to, AAV-DJ, AAV-HAE1, AAV-HAE2, AAVM41, AAV-1829, AAV2 Y/F, AAV2 T/V, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS, AAV-PHP.B, AAV-PHP.eB, AAV-PHP.S, AAV-F, AAVcc.47, and AAVcc.81. In an aspect, a disclosed AAV vector can be AAV-Rh74 or a related variant (e.g., capsid variants like RHM4-1). In an aspect, a disclosed AAV vector can be AAV8. In an aspect, a disclosed AAV vector can be AAVhum.8. In an aspect, a disclosed AAV vector can be a self-complementary AAV as disclosed herein.

In an aspect, a disclosed vector can comprise one or more promoters operably linked to a disclosed transgene, a disclosed sequence to be trans-spliced, a disclosed isolated nucleic acid molecule, a disclosed catalytically inactive Cas13 (e.g., RfxdCas13 or PspdCas13b), and/or a disclosed nucleic acid sequence. In an aspect, a disclosed promoter can be positioned 5′ (upstream) or 3′ (downstream) of a disclosed transgene, a disclosed sequence to be trans-spliced, a disclosed isolated nucleic acid molecule, a disclosed catalytically inactive Cas13 (e.g., RfxdCas13 or PspdCas13b), and/or a disclosed nucleic acid sequence under its control. The distance between a disclosed promoter and a disclosed transgene, a disclosed sequence to be trans-spliced, a disclosed isolated nucleic acid molecule, a disclosed catalytically inactive Cas13 (e.g., RfxdCas13 or PspdCas13b), and/or a disclosed nucleic acid sequence can be approximately the same as the distance between that promoter and to the disclosed transgene, the disclosed sequence to be trans-spliced, the disclosed isolated nucleic acid molecule, the disclosed catalytically inactive Cas13 (e.g., RfxdCas13 orPspdCas13b or Cas13 alternative), and/or the disclosed nucleic acid sequence under its control. As is known in the art, variation in this distance can be accommodated without loss of promoter function.

In an aspect, a disclosed promoter for the one or more disclosed isolated nucleic acid molecules or the one or more disclosed guide RNA sequences can be tissue-specific or ubiquitous and can be constitutive or inducible, depending on the pattern of the gene expression desired. A promoter can be native or foreign and can be a natural or a synthetic sequence. By foreign, it is intended that the transcriptional initiation region is not found in the wild-type host into which the transcriptional initiation region is introduced. In an aspect, a disclosed promoter can be a promoter/enhancer. In an aspect, a disclosed promoter for the one or more disclosed isolated nucleic acid molecules or the one or more disclosed guide RNA sequences can be an endogenous promoter. In an aspect, a disclosed endogenous promoter can be an endogenous promoter/enhancer. In an aspect, a disclosed endogenous promoter or a disclosed endogenous promoter/enhancer can generally be obtained from a non-coding region upstream of a transcription initiation site of a gene of interest. In an aspect, a disclosed endogenous promoter or a disclosed endogenous promoter/enhancer can be used for constitutive and efficient expression of a disclosed gene. In an aspect, a disclosed promoter for the one or more disclosed isolated nucleic acid molecules or the one or more disclosed guide RNA sequences can be a CMV promoter or a CMV promoter/enhancer. CMV promoters and CMV promoters/enhancers are well known to the art.

In an aspect, a disclosed promoter for the one or more disclosed guide RNA sequences can be any eukaryotic RNA polymerase II promoter.

### 4. Formulations

Disclosed herein is a pharmaceutical formulation comprising a disclosed isolated nucleic acid molecule. Disclosed herein is a pharmaceutical formulation comprising a disclosed isolated nucleic acid molecule and a pharmaceutically acceptable carrier. Disclosed herein is a pharmaceutical formulation comprising a disclosed vector. Disclosed herein is a pharmaceutical formulation comprising a disclosed vector and a pharmaceutically acceptable carrier.

Disclosed herein is a pharmaceutical formulation comprising a vector comprising an isolated nucleic acid molecule comprising a nucleic acid sequence to be trans-spliced to a target endogenous pre-mRNA; a 5′ hemi intron linked to the nucleic acid sequence to be trans-spliced; one or more guide RNA sequences; a promoter operably linked to the one or more guide RNA sequences; and a nucleic acid sequence encoding an RNA binding protein.

Disclosed herein is a pharmaceutical formulation comprising a vector comprising an isolated nucleic acid molecule comprising a nucleic acid sequence to be trans-spliced to a target endogenous pre-mRNA; a 3′ hemi intron; a 5′ hemi intron; two or more guide RNA sequences; a promoter operably linked to the two or more guide RNA sequences; and a nucleic acid sequence encoding one or more RNA binding proteins.

Disclosed herein is a pharmaceutical formulation comprising a vector comprising an isolated nucleic acid molecule comprising a nucleic acid sequence to be trans-spliced to a target endogenous pre-mRNA; a 3′ hemi intron linked to the nucleic acid sequence to be trans-spliced; one or more guide RNA sequences; a promoter operably linked to the one or more guide RNA sequences; and a nucleic acid sequence encoding an RNA binding protein.

Disclosed herein is a pharmaceutical formulation comprising a vector comprising an isolated nucleic acid molecule comprising a nucleic acid sequence encoding a catalytically inactive RfxCas13d; a promoter operably linked to the nucleic acid sequence encoding the catalytically inactive RfxCas13d; and a polyadenylation signal.

Disclosed herein is a pharmaceutical formulation comprising a vector comprising an isolated nucleic acid molecule comprising a nucleic acid sequence encoding a catalytically inactive PspdCas13b, a promoter operably linked to the nucleic acid sequence encoding the catalytically inactive PspdCas13b; and a polyadenylation signal.

Disclosed herein is a pharmaceutical formulation comprising a vector comprising an isolated nucleic acid molecule comprising a nucleic acid sequence encoding a Cas13 alternative, a promoter operably linked to the nucleic acid sequence encoding the Cas13 alternative; and a polyadenylation signal. In an aspect, a Cas13 alternative can comprise alternatives known to the art including, but not limited to CRISPR-Inspired RNA Targeting System (CIRTS), Pumillo and FBF (PUF), rCas9, and/or any bifunctional RNA binding protein that can both bind to the trans-splicing RNA molecule and the target RNA to facilitate interaction between the two RNA species. RNA binding proteins are discussed in depth supra.

Disclosed herein is a pharmaceutical formulation comprising a vector comprising an expression cassette comprising an isolated nucleic acid molecule comprising a nucleic acid sequence to be trans-spliced to a target endogenous pre-mRNA; a 5′ hemi intron linked to the nucleic acid sequence to be trans-spliced; one or more guide RNA sequences; a promoter operably linked to the one or more guide RNA sequences; and a nucleic acid sequence encoding an RNA binding protein.

Disclosed herein is a pharmaceutical formulation comprising a vector comprising an expression cassette comprising an isolated nucleic acid molecule comprising a nucleic acid sequence to be trans-spliced to a target endogenous pre-mRNA; a 3′ hemi intron linked to the nucleic acid sequence to be trans-spliced; one or more guide RNA sequences; a promoter operably linked to the one or more guide RNA sequences; and a nucleic acid sequence encoding an RNA binding protein.

Disclosed herein is a pharmaceutical formulation comprising a vector comprising an expression cassette comprising an isolated nucleic acid molecule comprising a nucleic acid sequence to be trans-spliced to a target endogenous pre-mRNA; a 3′ hemi intron; a 5′ hemi intron; two or more guide RNA sequences; a promoter operably linked to the two or more guide RNA sequences; and a nucleic acid sequence encoding one or more RNA binding proteins.

Disclosed herein is a pharmaceutical formulation comprising a vector comprising an expression cassette comprising an isolated nucleic acid molecule, comprising a nucleic acid sequence encoding a catalytically inactive RfxCas13d; a promoter operably linked to the nucleic acid sequence encoding the catalytically inactive RfxCas13d; and a polyadenylation signal.

Disclosed herein is a pharmaceutical formulation comprising a vector comprising an expression cassette an isolated nucleic acid molecule, comprising a nucleic acid sequence encoding a catalytically inactive PspdCas13b, a promoter operably linked to the nucleic acid sequence encoding the catalytically inactive PspdCas13b; and a polyadenylation signal.

Disclosed herein is a pharmaceutical formulation comprising a vector comprising an expression cassette an isolated nucleic acid molecule, comprising a nucleic acid sequence encoding a Cas13 alternative, a promoter operably linked to the nucleic acid sequence encoding the Cas13 alternative; and a polyadenylation signal. In an aspect, a Cas13 alternative can comprise alternatives known to the art including, but not limited to CRISPR-Inspired RNA Targeting System (CIRTS), Pumillo and FBF (PUF), rCas9, and/or any bifunctional RNA binding protein that can both bind to the trans-splicing RNA molecule and the target RNA to facilitate interaction between the two RNA species. RNA binding proteins are discussed in depth supra.

In an aspect, a disclosed pharmaceutical formulation can restore one or more aspects of cellular homeostasis and/or cellular functionality and/or metabolic dysregulation. In an aspect, a disclosed pharmaceutical formulation can restore the functionality and/or structural integrity of a missing, deficient, and/or mutant protein or enzyme.

In an aspect, a disclosed formulation can comprise (i) one or more active agents, (ii) biologically active agents, (iii) one or more pharmaceutically active agents, (iv) one or more immune-based therapeutic agents, (v) one or more clinically approved agents, or (vi) a combination thereof. In an aspect, a disclosed composition can comprise one or more immune modulators. In an aspect, a disclosed composition can comprise one or more proteasome inhibitors. In an aspect, a disclosed composition can comprise one or more immunosuppressives or immunosuppressive agents. In an aspect, an immunosuppressive agent can be anti-thymocyte globulin (ATG), cyclosporine (CSP), mycophenolate mofetil (MMF), or a combination thereof. In an aspect, a disclosed formulation can comprise an anaplerotic agent (such as, for example, C7 compounds like triheptanoin or MCT).

In an aspect, a disclosed formulation can comprise an RNA therapeutic. An RNA therapeutic can comprise RNA-mediated interference (RNAi) and/or antisense oligonucleotides (ASO). In an aspect, a disclosed RNA therapeutic can be directed at any protein or enzyme that is overexpressed or is overactive due to a missing, deficient, and/or mutant protein or enzyme. In an aspect, a disclosed RNA therapeutic can comprise therapy delivered via LNPs. In an aspect, a disclosed formulation can comprise an enzyme or enzyme precursor for enzyme replacement therapy (ERT).

In an aspect, a disclosed formulation can comprise a disclosed small molecule. In an aspect, a disclosed small molecule can assist in restoring the functionality and/or structural integrity of a missing, deficient, and/or mutant protein or enzyme.

In an aspect, any disclosed pharmaceutical formulation can comprise one or more excipients and/or pharmaceutically acceptable carriers. Excipients and/or pharmaceutically acceptable carriers are known to the art and are discussed supra.

### 5. Plasmids

Disclosed herein is a plasmid comprising one or more disclosed isolated nucleic acid molecules. Disclosed herein is a plasmid comprising one or more disclosed vectors. Disclosed here are plasmids used in methods of making a disclosed composition such as, for example, a disclosed isolated nucleic acid molecule, a disclosed vector, or a disclosed pharmaceutical formulation. Plasmids and using plasmids are known to the art.

Disclosed herein is a plasmid comprising the sequence set forth in any one of SEQ ID NO:01—SEQ ID NO:22 or a fragment thereof. Disclosed herein is a plasmid comprising a sequence having at least 40%, 50%, 60%, 70%, 80%, 85%, 86%, 87%, 88%, 89%, 90%, 91%, 92%, 93%, 94%, 95%, 96%, 97%, 98%, 99% identity to the sequence set forth in any one of SEQ ID NO:01—SEQ ID NO:22 or a fragment thereof. Disclosed herein is a plasmid comprising a sequence having at least 40%-60%, at least 60%-80%, at least 80%-90%, or at least 90%-100% identity to the sequence set forth in any one of SEQ ID NO:01—SEQ ID NO:22 or a fragment thereof.

### 6. Cells

Disclosed herein are cells comprising a disclosed isolated nucleic acid molecule, a disclosed vector, and/or a disclosed plasmid. Disclosed herein are cells transduced by one or more disclosed viral vectors. Disclosed herein are cells transfected with one or more disclosed isolated nucleic acid molecules. In an aspect, a disclosed cell has been transfected with one or more nucleic acid sequences having the sequence set forth in any of SEQ ID NO:01—SEQ ID NO:22. Techniques to achieve transfection and transduction are known to the art and using transfected or transduced cells are known to the art. In an aspect, disclosed herein are human immortalized cells lines transduced by one or more disclosed viral vectors or transfected with one or more disclosed isolated nucleic acids or disclosed plasmids. In an aspect, disclosed herein are human immortalized cells lines contacted with one or more disclosed pharmaceutical formulations. Disclosed herein are cells obtained for a subject treated with one or more disclosed isolated nucleic acid molecule, one or more disclosed vectors, one or more disclosed plasmids, or one or more disclosed pharmaceutical formulations.

### 7. Animals

Disclosed herein are animals treated with one or more disclosed isolated nucleic acid molecules, one or more disclosed vectors, one or more disclosed pharmaceutical formulations, and/or one or more disclosed plasmids (e.g., SEQ ID NO:01-SEQ ID NO:22). Transgenic animals are known to the art as are the techniques to generate transgenic animals.

### C. Methods of Generating a Chimeric RNA Molecule

Disclosed herein is a method of generating a chimeric RNA molecule in a cell, the method comprising contacting an endogenous pre-mRNA in a cell with (i) an isolated nucleic acid molecule comprising a nucleic acid sequence to be trans-spliced to a target endogenous pre-mRNA; a 5′ hemi intron linked to the nucleic acid sequence to be trans-spliced; one or more guide RNA sequences; a promoter operably linked to the one or more guide RNA sequences; and a nucleic acid sequence encoding an RNA binding protein, and (ii) an isolated nucleic acid molecule, comprising a nucleic acid sequence encoding a catalytically inactive PspdCas13b, a promoter operably linked to the nucleic acid sequence encoding the catalytically inactive PspdCas13b; and a polyadenylation signal, wherein the isolated nucleic acid molecules form a ternary complex with the endogenous pre-mRNA molecule, and wherein the resulting chimeric RNA molecule comprises the trans-spliced nucleic acid sequence.

Disclosed herein is a method of generating a chimeric RNA molecule in a cell, the method comprising contacting an endogenous pre-mRNA in a cell with (i) an isolated nucleic acid molecule comprising a nucleic acid sequence to be trans-spliced to a target endogenous pre-mRNA; a 3′ hemi intron; a 5′ hemi intron; two or more guide RNA sequences; a promoter operably linked to the two or more guide RNA sequences; and a nucleic acid sequence encoding one or more RNA binding proteins; and (ii) an isolated nucleic acid molecule, comprising a nucleic acid sequence encoding a catalytically inactive RfxCas13d; a promoter operably linked to the nucleic acid sequence encoding the catalytically inactive RfxCas13d; and a polyadenylation signal, wherein the isolated nucleic acid molecules form a ternary complex with the endogenous pre-mRNA molecule, and wherein the resulting chimeric RNA molecule comprises the trans-spliced nucleic acid sequence.

Disclosed herein is a method of generating a chimeric RNA molecule in a cell, the method comprising contacting an endogenous pre-mRNA in a cell with (i) an isolated nucleic acid molecule comprising a nucleic acid sequence to be trans-spliced to a target endogenous pre-mRNA; a 3′ hemi intron linked to the nucleic acid sequence to be trans-spliced; one or more guide RNA sequences; a promoter operably linked to the one or more guide RNA sequences; and a nucleic acid sequence encoding an RNA binding protein; and (ii) an isolated nucleic acid molecule, comprising a nucleic acid sequence encoding a catalytically inactive RfxCas13d; a promoter operably linked to the nucleic acid sequence encoding the catalytically inactive RfxCas13d; and a polyadenylation signal, wherein the isolated nucleic acid molecules form a ternary complex with the endogenous pre-mRNA molecule, and wherein the resulting chimeric RNA molecule comprises the trans-spliced nucleic acid sequence.

Disclosed herein is a method of generating a chimeric RNA molecule in a cell, the method comprising contacting an endogenous pre-mRNA in a cell with (i) an isolated nucleic acid molecule comprising a nucleic acid sequence to be trans-spliced to a target endogenous pre-mRNA; a 5′ hemi intron linked to the nucleic acid sequence to be trans-spliced; one or more guide RNA sequences; a promoter operably linked to the one or more guide RNA sequences; and a nucleic acid sequence encoding an RNA binding protein, and (ii) an isolated nucleic acid molecule, comprising a nucleic acid sequence encoding a Cas13 alternative, a promoter operably linked to the nucleic acid sequence encoding the Cas13 alternative; and a polyadenylation signal, wherein the isolated nucleic acid molecules form a ternary complex with the endogenous pre-mRNA molecule, and wherein the resulting chimeric RNA molecule comprises the trans-spliced nucleic acid sequence.

Disclosed herein is a method of generating a chimeric RNA molecule in a cell, the method comprising contacting an endogenous pre-mRNA in a cell with (i) an isolated nucleic acid molecule comprising a nucleic acid sequence to be trans-spliced to a target endogenous pre-mRNA; a 3′ hemi intron; a 5′ hemi intron; two or more guide RNA sequences; a promoter operably linked to the two or more guide RNA sequences; and a nucleic acid sequence encoding one or more RNA binding proteins; and (ii) an isolated nucleic acid molecule, comprising a nucleic acid sequence encoding a Cas13 alternative; a promoter operably linked to the nucleic acid sequence encoding the Cas13 alternative; and a polyadenylation signal, wherein the isolated nucleic acid molecules form a ternary complex with the endogenous pre-mRNA molecule, and wherein the resulting chimeric RNA molecule comprises the trans-spliced nucleic acid sequence.

Disclosed herein is a method of generating a chimeric RNA molecule in a cell, the method comprising contacting an endogenous pre-mRNA in a cell with (i) an isolated nucleic acid molecule comprising a nucleic acid sequence to be trans-spliced to a target endogenous pre-mRNA; a 3′ hemi intron linked to the nucleic acid sequence to be trans-spliced; one or more guide RNA sequences; a promoter operably linked to the one or more guide RNA sequences; and a nucleic acid sequence encoding an RNA binding protein; and (ii) an isolated nucleic acid molecule, comprising a nucleic acid sequence encoding a Cas13 alternative; a promoter operably linked to the nucleic acid sequence encoding the Cas13 alternative; and a polyadenylation signal, wherein the isolated nucleic acid molecules form a ternary complex with the endogenous pre-mRNA molecule, and wherein the resulting chimeric RNA molecule comprises the trans-spliced nucleic acid sequence.

In an aspect, a Cas13 alternative can comprise alternatives known to the art including, but not limited to CRISPR-Inspired RNA Targeting System (CIRTS), Pumillo and FBF (PUF), rCas9, and/or any bifunctional RNA binding protein that can both bind to the trans-splicing RNA molecule and the target RNA to facilitate interaction between the two RNA species. RNA binding proteins are discussed in depth supra.

In an aspect, the cell can be in a subject. In an aspect, a subject can be diagnosed with or can be suspected of having a genetic disease or disorder. In an aspect, a disease or disorder can comprise any disease or disorder caused by a disclosed gene or a missing, deficient, and/or mutant gene. In an aspect, a subject can be a subject in need of treatment of a disclosed disease or disorder (e.g., a genetic disease or disorder). Genetic diseases and disorders are discussed extensively herein.

In an aspect, a disclosed method of generating a chimeric RNA molecule in a cell can comprise validating the trans-splicing and/or the generation of the chimeric RNA molecule. Validation of the trans-splicing event and/or generation of the chimeric RNA molecule can be accomplished using methods and techniques known to the art (e.g., sequencing, northern blots, FISH, PCR, RNA-Seq, 3′ RACE, 5′ RACE, etc.).

### D. Methods of Treating and/or Preventing a Genetic Disease or Disorder

Disclosed herein is a method of treating and/or preventing a genetic disease or disorder, the method comprising generating a chimeric RNA molecule in one or more cells by administering to a subject in need thereof (i) an isolated nucleic acid molecule comprising a nucleic acid sequence to be trans-spliced to a target endogenous pre-mRNA; a 5′ hemi intron linked to the nucleic acid sequence to be trans-spliced; one or more guide RNA sequences; a promoter operably linked to the one or more guide RNA sequences; and a nucleic acid sequence encoding an RNA binding protein, and (ii) an isolated nucleic acid molecule comprising a nucleic acid sequence encoding a catalytically inactive PspdCas13b, a promoter operably linked to the nucleic acid sequence encoding the catalytically inactive PspdCas13b; and a polyadenylation signal, wherein the resulting chimeric RNA molecule comprises the trans-spliced nucleic acid sequence; and wherein the resulting chimeric RNA molecule can restore one or more aspects of cellular homeostasis and/or cellular functionality and/or metabolic dysregulation.

Disclosed herein is a method of treating and/or preventing a genetic disease or disorder, the method comprising generating a chimeric RNA molecule in one or more cells by administering to a subject in need thereof (i) an isolated nucleic acid molecule comprising a nucleic acid sequence to be trans-spliced to a target endogenous pre-mRNA; a 3′ hemi intron; a 5′ hemi intron; two or more guide RNA sequences; a promoter operably linked to the two or more guide RNA sequences; and a nucleic acid sequence encoding one or more RNA binding proteins; and (ii) an isolated nucleic acid molecule comprising a nucleic acid sequence encoding a catalytically inactive RfxCas13d; a promoter operably linked to the nucleic acid sequence encoding the catalytically inactive RfxCas13d; and a polyadenylation signal, wherein the resulting chimeric RNA molecule comprises the trans-spliced nucleic acid sequence; and wherein the resulting chimeric RNA molecule can restore one or more aspects of cellular homeostasis and/or cellular functionality and/or metabolic dysregulation.

Disclosed herein is a method of treating and/or preventing a genetic disease or disorder, the method comprising generating a chimeric RNA molecule in one or more cells by administering to a subject in need thereof (i) an isolated nucleic acid molecule comprising a nucleic acid sequence to be trans-spliced to a target endogenous pre-mRNA; a 3′ hemi intron linked to the nucleic acid sequence to be trans-spliced; one or more guide RNA sequences; a promoter operably linked to the one or more guide RNA sequences; and a nucleic acid sequence encoding an RNA binding protein; and (ii) an isolated nucleic acid molecule, comprising a nucleic acid sequence encoding a catalytically inactive RfxCas13d; a promoter operably linked to the nucleic acid sequence encoding the catalytically inactive RfxCas13d; and a polyadenylation signal, wherein the resulting chimeric RNA molecule comprises the trans-spliced nucleic acid sequence; and wherein the resulting chimeric RNA molecule can restore one or more aspects of cellular homeostasis and/or cellular functionality and/or metabolic dysregulation.

Disclosed herein is a method of treating and/or preventing a genetic disease or disorder, the method comprising generating a chimeric RNA molecule in one or more cells by administering to a subject in need thereof (i) a vector comprising an isolated nucleic acid molecule comprising a nucleic acid sequence to be trans-spliced to a target endogenous pre-mRNA; a 5′ hemi intron linked to the nucleic acid sequence to be trans-spliced; one or more guide RNA sequences; a promoter operably linked to the one or more guide RNA sequences; and a nucleic acid sequence encoding an RNA binding protein, and (ii) a vector comprising an isolated nucleic acid molecule comprising a nucleic acid sequence encoding a catalytically inactive PspdCas13b, a promoter operably linked to the nucleic acid sequence encoding the catalytically inactive PspdCas13b; and a polyadenylation signal, wherein the resulting chimeric RNA molecule comprises the trans-spliced nucleic acid sequence; and wherein the resulting chimeric RNA molecule can restore one or more aspects of cellular homeostasis and/or cellular functionality and/or metabolic dysregulation.

Disclosed herein is a method of treating and/or preventing a genetic disease or disorder, the method comprising generating a chimeric RNA molecule in one or more cells by administering to a subject in need thereof (i) a vector comprising an isolated nucleic acid molecule comprising a nucleic acid sequence to be trans-spliced to a target endogenous pre-mRNA; a 3′ hemi intron; a 5′ hemi intron; two or more guide RNA sequences; a promoter operably linked to the two or more guide RNA sequences; and a nucleic acid sequence encoding one or more RNA binding proteins; and (ii) a vector comprising an isolated nucleic acid molecule comprising a nucleic acid sequence encoding a catalytically inactive RfxCas13d; a promoter operably linked to the nucleic acid sequence encoding the catalytically inactive RfxCas13d; and a polyadenylation signal, wherein the resulting chimeric RNA molecule comprises the trans-spliced nucleic acid sequence; and wherein the resulting chimeric RNA molecule can restore one or more aspects of cellular homeostasis and/or cellular functionality and/or metabolic dysregulation.

Disclosed herein is a method of treating and/or preventing a genetic disease or disorder, the method comprising generating a chimeric RNA molecule in one or more cells by administering to a subject in need thereof (i) a vector comprising an isolated nucleic acid molecule comprising a nucleic acid sequence to be trans-spliced to a target endogenous pre-mRNA; a 3′ hemi intron linked to the nucleic acid sequence to be trans-spliced; one or more guide RNA sequences; a promoter operably linked to the one or more guide RNA sequences; and a nucleic acid sequence encoding an RNA binding protein; and (ii) a vector comprising an isolated nucleic acid molecule, comprising a nucleic acid sequence encoding a catalytically inactive RfxCas13d; a promoter operably linked to the nucleic acid sequence encoding the catalytically inactive RfxCas13d; and a polyadenylation signal, wherein the resulting chimeric RNA molecule comprises the trans-spliced nucleic acid sequence; and wherein the resulting chimeric RNA molecule can restore one or more aspects of cellular homeostasis and/or cellular functionality and/or metabolic dysregulation.

Disclosed herein is a method of treating and/or preventing a genetic disease or disorder, the method comprising generating a chimeric RNA molecule in one or more cells by administering to a subject in need thereof (i) an isolated nucleic acid molecule comprising a nucleic acid sequence to be trans-spliced to a target endogenous pre-mRNA; a 5′ hemi intron linked to the nucleic acid sequence to be trans-spliced; one or more guide RNA sequences; a promoter operably linked to the one or more guide RNA sequences; and a nucleic acid sequence encoding an RNA binding protein, and (ii) an isolated nucleic acid molecule comprising a nucleic acid sequence encoding a Cas13 alternative, a promoter operably linked to the nucleic acid sequence encoding the Cas13 alternative; and a polyadenylation signal, wherein the resulting chimeric RNA molecule comprises the trans-spliced nucleic acid sequence; and wherein the resulting chimeric RNA molecule can restore one or more aspects of cellular homeostasis and/or cellular functionality and/or metabolic dysregulation.

Disclosed herein is a method of treating and/or preventing a genetic disease or disorder, the method comprising generating a chimeric RNA molecule in one or more cells by administering to a subject in need thereof (i) an isolated nucleic acid molecule comprising a nucleic acid sequence to be trans-spliced to a target endogenous pre-mRNA; a 3′ hemi intron; a 5′ hemi intron; two or more guide RNA sequences; a promoter operably linked to the two or more guide RNA sequences; and a nucleic acid sequence encoding one or more RNA binding proteins; and (ii) an isolated nucleic acid molecule comprising a nucleic acid sequence encoding a Cas13 alternative; a promoter operably linked to the nucleic acid sequence encoding the Cas13 alternative; and a polyadenylation signal, wherein the resulting chimeric RNA molecule comprises the trans-spliced nucleic acid sequence; and wherein the resulting chimeric RNA molecule can restore one or more aspects of cellular homeostasis and/or cellular functionality and/or metabolic dysregulation.

Disclosed herein is a method of treating and/or preventing a genetic disease or disorder, the method comprising generating a chimeric RNA molecule in one or more cells by administering to a subject in need thereof (i) an isolated nucleic acid molecule comprising a nucleic acid sequence to be trans-spliced to a target endogenous pre-mRNA; a 3′ hemi intron linked to the nucleic acid sequence to be trans-spliced; one or more guide RNA sequences; a promoter operably linked to the one or more guide RNA sequences; and a nucleic acid sequence encoding an RNA binding protein; and (ii) an isolated nucleic acid molecule, comprising a nucleic acid sequence encoding a Cas13 alternative; a promoter operably linked to the nucleic acid sequence encoding the Cas13 alternative; and a polyadenylation signal, wherein the resulting chimeric RNA molecule comprises the trans-spliced nucleic acid sequence; and wherein the resulting chimeric RNA molecule can restore one or more aspects of cellular homeostasis and/or cellular functionality and/or metabolic dysregulation.

Disclosed herein is a method of treating and/or preventing a genetic disease or disorder, the method comprising generating a chimeric RNA molecule in one or more cells by administering to a subject in need thereof (i) a vector comprising an isolated nucleic acid molecule comprising a nucleic acid sequence to be trans-spliced to a target endogenous pre-mRNA; a 5′ hemi intron linked to the nucleic acid sequence to be trans-spliced; one or more guide RNA sequences; a promoter operably linked to the one or more guide RNA sequences; and a nucleic acid sequence encoding an RNA binding protein, and (ii) a vector comprising an isolated nucleic acid molecule comprising a nucleic acid sequence encoding a Cas13 alternative, a promoter operably linked to the nucleic acid sequence encoding the Cas13 alternative; and a polyadenylation signal, wherein the resulting chimeric RNA molecule comprises the trans-spliced nucleic acid sequence; and wherein the resulting chimeric RNA molecule can restore one or more aspects of cellular homeostasis and/or cellular functionality and/or metabolic dysregulation.

Disclosed herein is a method of treating and/or preventing a genetic disease or disorder, the method comprising generating a chimeric RNA molecule in one or more cells by administering to a subject in need thereof (i) a vector comprising an isolated nucleic acid molecule comprising a nucleic acid sequence to be trans-spliced to a target endogenous pre-mRNA; a 3′ hemi intron; a 5′ hemi intron; two or more guide RNA sequences; a promoter operably linked to the two or more guide RNA sequences; and a nucleic acid sequence encoding one or more RNA binding proteins; and (ii) a vector comprising an isolated nucleic acid molecule comprising a nucleic acid sequence encoding a Cas13 alternative; a promoter operably linked to the nucleic acid sequence encoding the Cas13 alternative; and a polyadenylation signal, wherein the resulting chimeric RNA molecule comprises the trans-spliced nucleic acid sequence; and wherein the resulting chimeric RNA molecule can restore one or more aspects of cellular homeostasis and/or cellular functionality and/or metabolic dysregulation.

Disclosed herein is a method of treating and/or preventing a genetic disease or disorder, the method comprising generating a chimeric RNA molecule in one or more cells by administering to a subject in need thereof (i) a vector comprising an isolated nucleic acid molecule comprising a nucleic acid sequence to be trans-spliced to a target endogenous pre-mRNA; a 3′ hemi intron linked to the nucleic acid sequence to be trans-spliced; one or more guide RNA sequences; a promoter operably linked to the one or more guide RNA sequences; and a nucleic acid sequence encoding an RNA binding protein; and (ii) a vector comprising an isolated nucleic acid molecule, comprising a nucleic acid sequence encoding a Cas13 alternative; a promoter operably linked to the nucleic acid sequence encoding the Cas13 alternative; and a polyadenylation signal, wherein the resulting chimeric RNA molecule comprises the trans-spliced nucleic acid sequence; and wherein the resulting chimeric RNA molecule can restore one or more aspects of cellular homeostasis and/or cellular functionality and/or metabolic dysregulation.

In an aspect, a Cas13 alternative can comprise alternatives known to the art including, but not limited to CRISPR-Inspired RNA Targeting System (CIRTS), Pumillo and FBF (PUF), rCas9, and/or any bifunctional RNA binding protein that can both bind to the trans-splicing RNA molecule and the target RNA to facilitate interaction between the two RNA species. RNA binding proteins are discussed in depth supra.

In an aspect, a subject can have or be suspected of having a disease or disorder that can be treated with gene therapy. Examples of such diseases or disorder can include, but are not limited to: cystic fibrosis (cystic fibrosis transmembrane regulator protein) and other diseases of the lung, hemophilia A (Factor VIII), hemophilia B (Factor IX), thalassemia (β-globin), anemia (erythropoietin) and other blood disorders, Alzheimer's disease (GDF; neprilysin), multiple sclerosis (β-interferon), Parkinson's disease (glial-cell line derived neurotrophic factor [GDNF]), Huntington's disease (RNAi to remove repeats), amyotrophic lateral sclerosis, epilepsy (galanin, neurotrophic factors), and other neurological disorders, cancer (endostatin, angiostatin, TRAIL, FAS-ligand, cytokines including interferons; RNAi including RNAi against VEGF or the multiple drug resistance gene product, mir-26a [e.g., for hepatocellular carcinoma]), diabetes mellitus (insulin), muscular dystrophies including Duchenne (dystrophin, mini-dystrophin, insulin-like growth factor I, a sarcoglycan [e.g., α, β, γ], RNAi against myostatin, myostatin propeptide, follistatin, activin type II soluble receptor, anti-inflammatory polypeptides such as the I-kappa B dominant mutant, sarcospan, utrophin, mini-utrophin, antisense or RNAi against splice junctions in the dystrophin gene to induce exon skipping (see, e.g., WO 2003/095647), antisense against U7 snRNAs to induce exon skipping (see, e.g., WO 2006/021724), and antibodies or antibody fragments against myostatin or myostatin propeptide) and Becker, Gaucher disease (glucocerebrosidase), Hurler's disease (α-L-iduronidase), adenosine deaminase deficiency (adenosine deaminase), glycogen storage diseases (e.g., Fabry disease [α-galactosidase] and Pompe disease [lysosomal acid α-glucosidase]) and other metabolic disorders, congenital emphysema (al-antitrypsin), Lesch-Nyhan Syndrome (hypoxanthine guanine phosphoribosyl transferase), Niemann-Pick disease (sphingomyelinase), Tay Sachs disease (lysosomal hexosaminidase A), Maple Syrup Urine Disease (branched-chain keto acid dehydrogenase), retinal degenerative diseases (and other diseases of the eye and retina; e.g., PDGF for macular degeneration and/or vasohibin or other inhibitors of VEGF or other angiogenesis inhibitors to treat/prevent retinal disorders, e.g., in Type I diabetes), diseases of solid organs such as brain (including Parkinson's Disease [GDNF], astrocytomas [endostatin, angiostatin and/or RNAi against VEGF], glioblastomas [endostatin, angiostatin and/or RNAi against VEGF]), liver, kidney, heart including congestive heart failure or peripheral artery disease (PAD) (e.g., by delivering protein phosphatase inhibitor I (I-1) and fragments thereof (e.g., IIC), serca2a, zinc finger proteins that regulate the phospholamban gene, Barkct, P2-adrenergic receptor, p2-adrenergic receptor kinase (BARK), phosphoinositide-3 kinase (PI3 kinase), S100A1, parvalbumin, adenylyl cyclase type 6, a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, etc.), arthritis (insulin-like growth factors), joint disorders (insulin-like growth factor 1 and/or 2), intimal hyperplasia (e.g., by delivering enos, inos), improve survival of heart transplants (superoxide dismutase), AIDS (soluble CD4), muscle wasting (insulin-like growth factor I), kidney deficiency (erythropoietin), anemia (erythropoietin), arthritis (anti-inflammatory factors such as IRAP and TNFa soluble receptor), hepatitis (a-interferon), LDL receptor deficiency (LDL receptor), hyperammonemia (ornithine transcarbamylase), Krabbe's disease (galactocerebrosidase), Batten's disease, spinal cerebral ataxias including SCA1, SCA2 and SCA3, phenylketonuria (phenylalanine hydroxylase), autoimmune diseases, and the like.

Genetic diseases and disorders are discussed supra and include, but are not limited to, diseases and disorders due to a defect in the following genes: ABCA1, ABCA12, ABCA13, ABCA2, ABCA3, ABCA4, ABCA5, ABCC1, ABCC2, ABCC6, ABCC8, ABCC9, ACAN, ADAMTS13, ADCY10, ADGRV1, AGL, AGRN, AHDC1, ALK, ALMS1, ALPK3, ALS2, ANAPC1, ANK1, ANK2, ANK3, ANKRD11, ANKRD26, APC, APC2, APOB, ARFGEF2, ARHGAP31, ARHGEF10, ARHGEF18, ARID1A, ARIDIB, ARID2, ASH1L, ASPM, ASXL1, ASXL2, ASXL3, ATM, ATP7A, ATP7B, ATR, ATRX, BAZ1A, BAZ2B, BCOR, BCORL1, BDP1, BLM, BPTF, BRCA1, BRCA2, BRD4, BRWD3, C2CD3, C3, C5, CACNA1A, CACNA1B, CACNA1C, CACNA1D, CACNA1E, CACNA1F, CACNA1G, CACNA1H, CACNA1S, CAD, CAMTA1, CARMIL2, CC2D2A, CCDC88A, CCDC88C, CCNB3, CDH23, CDK13, CDK5RAP2, CELSR1, CEMIP2, CENPE, CENPF, CENPJ, CEP152, CEP164, CEP250, CEP290, CFAP43, CFAP44, CFAP65, CFTR/ABCC7, CHD1, CHD2, CHD3, CHD4, CHD7, CHD8, CIC, CIT, CLIP1, CLTC, CNOT1, CNTNAP1, COL11A1, COL11A2, COL12A1, COL17A1, COL18A1, COL1A1, COL1A2, COL27A1, COL2A1, COL3A1, COL4A1, COL4A2, COL4A3, COL4A4, COL4A5, COL4A6, COL5A1, COL5A2, COL6A3, COL7A1, CPAMD8, CPLANEl, CPS1, CPSF1, CRB1, CREBBP, CUBN, CUL7, CUX1, DCC, DCHS1, DEPDC5, DICERI, DIP2B, DLC1, DMD, DMXL2, DNAH1, DNAH11, DNAH17, DNAH2, DNAH5, DNAH7, DNAH8, DNAH9, DNMBP, DNMT1, DOCK2, DOCK3, DOCK6, DOCK7, DOCK8, DSCAM, DSP, DST, DUOX2, DYNCIHI, DYNC2H1, DYSF, EIF2AK4, EP300, EPG5, ERCC6, ERCC6L2, EXPH5, EYS, F5, F8, FANCA, FANCD2, FANCM, FAT1, FAT4, FBN1, FBN2, FLG, FLG2, FLNA, FLNB, FLNC, FLT4, FMN2, FN1, FRAS1, FREM1, FREM2, FSIP2, FYCO1, GLI2, GLI3, GPR179, GREBIL, GRIN2A, GRIN2B, GRIN2D, HCFC1, HECW2, HERC1, HERC2, HFM1, HIVEP1, HIVEP2, HMCN1, HSPG2, HTT, HUWE1, HYDIN, IFT140, IFT172, IGF1R, IGF2R, IGSF1, INSR, INTS1, IQSEC2, ITGB4, ITPR1, ITPR2, JMJD1C, KALRN, KANK1, KAT6A, KAT6B, KDM3B, KDM5B, KDM5C, KDM6A, KDM6B, KDR, KIAA0586, KIAA1109, KIAA1549, KIDINS220, KIF14, KIF1A, KIF1B, KIF21A, KIF26B, KIF7, KMT2A, KMT2B, KMT2C, KMT2D, KMT2E, KNL1, LAMA1, LAMA2, LAMA3, LAMA4, LAMA5, LAMB1, LAMB2, LAMC3, LCT, LOXHD1, LPA, LRBA, LRP1, LRP2, LRP4, LRP5, LRP6, LRPPRC, LRRK1, LRRK2, LTBP2, LTBP4, LYST, MACF1, MADD, MAGI2, MAP1B, MAP3K1, MAPK8IP3, MAPKBP1, MAST1, MBD5, MCM3AP, MED12, MED12L, MED13, MED13L, MED23, MEGF8, MET, MLH3, MPDZ, MSH6, MTOR, MYH10, MYH11, MYH14, MYH2, MYH3, MYH6, MYH7, MYH7B, MYH8, MYH9, MYLK, MYO15A, MYO18B, MYO3A, MYOSA, MYO5B, MYO7A, MYO9A, NALCN, NBAS, NBEA, NBEAL2, NCAPD2, NCAPD3, NEB, NEXMIF, NEXMIF, NF1, NFASC, NHS, NIN, NIPBL, NLRP1, NOTCH1, NOTCH2, NOTCH3, NPHP4, NRXN1, NRXN3, NSD1, NSD2, NUP155, NUP188, NUP205, OBSCN, OBSL1, OTOF, OTOG, OTOGL, PARD3, PBRM1, PCDH15, PCLO, PCNT, PHIP, PI4KA, PIEZO1, PIEZO2, PIK3C2A, PIKFYVE, PKD1, PKD1L1, PKHD1, PLCE1, PLEC, PLEKHG2, PNPLA6, POGZ, POLA1, POLE, POLR1A, POLR2A, POLR3A, PRG4, PRKDC, PRPF8, PRR12, PRX, PTCH1, PTPN23, PTPRF, PTPRJ, PTPRQ, PXDN, QRICH2, RAB3GAP2, RAIL, RALGAPA1, RANBP2, RB1CC1, RELN, RERE, REV3L, RIC1, RIMS1, RIMS2, RNF213, ROBO1, ROBO2, ROBO3, ROS1, RP1, RP1L1, RTTN, RUSC2, RYR1, RYR2, SACS, SAMD9, SAMD9L, SBF2, SCAPER, SCN10A, SCN11A, SCN1A, SCN2A, SCN3A, SCN4A, SCN5A, SCN8A, SCN9A, SETBP1, SETD1A, SETD1B, SETD2, SETD5, SETX, SHANK2, SHANK3, SHROOM4, SI, SIPA1L3, SLIT2, SLX4, SMARCA2, SMARCA4, SMCHD1, SNRNP200, SON, SPEF2, SPEG, SPG11, SPTA1, SPTAN1, SPTB, SPTBN2, SPTBN4, SRCAP, STRC, SVIL, SYNE1, SYNGAP1, SYNJ1, SZT2, TAF1, TANC2, TCF20, TCOF1, TDRD9, TECPR2, TECTA, TENM3, TENM4, TET3, TEX14, TEX15, TG, THOC2, TMEM94, TNC, TNIK, TNR, TNRC6B, TNXB, TOGARAMI, TONSL, TRIO, TRIOBP, TRIP11, TRIP12, TRPM1, TRPM6, TRPM7, TRRAP, TSC2, TTC37, TTN, TUBGCP6, UBR1, UNC80, USH2A, USP9X, VCAN, VPS13A, VPS13B, VPS13C, VPS13D, VWF, WDFY3, WDR19, WDR62, WDR81, WNK1, WRN, ZFHX2, ZFYVE26, ZNF142, ZNF292, ZNF335, ZNF407, ZNF462, ZNF469, or a portion thereof.

Genetic diseases and disorders can also include, but are not limited to, diseases and disorders due to a defect in the following gene:: dystrophin including mini- and micro-dystrophins (DMD); titin (TTN); titin cap (TCAP) α-sarcoglycan (SGCA), β-sarcoglycan (SGCB), γ-sarcoglycan (SGCG) or 6-sarcoglycan (SGCD); alpha-1-antitrypsin (A1-AT); myosin heavy chain 6 (MYH6); myosin heavy chain 7 (MYH7); myosin heavy chain 11 (MYH11); myosin light chain 2 (ML2); myosin light chain 3 (ML3); myosin light chain kinase 2 (MYLK2); myosin binding protein C (MYBPC3); desmin (DES); dynamin 2 (DNM2); laminin α2 (LAMA2); lamin A/C (LMNA); lamin B (LMNB); lamin B receptor (LBR); dysferlin (DYSF); emerin (EMD); insulin; blood clotting factors, including but not limited to, factor VIII and factor IX; erythropoietin (EPO); lipoprotein lipase (LPL); sarcoplasmic reticulum Ca2++-ATPase (SERCA2A), S100 calcium binding protein A1 (S100A1); myotubularin (MTM); DM1 protein kinase (DMPK); glycogen phosphorylase L (PYGL); glycogen phosphorylase, muscle associated (PYGM); glycogen synthase 1 (GYS1); glycogen synthase 2 (GYS2); α-galactosidase A (GLA); α-N-acetylgalactosaminidase (NAGA); acid α-glucosidase (GAA), sphingomyelinase phosphodiesterase 1 (SMPD1); lysosomal acid lipase (LIPA); collagen type I α1 chain (COL1A1); collagen type I α2 chain (COL1A2); collagen type III α1 chain (COL3A1); collagen type V al chain (COL5A1); collagen type V α2 chain (COL5A2); collagen type VI α1 chain (COL6A1); collagen type VI α2 chain (COL6A2); collagen type VI 3 chain (COL6A3); procollagen-lysine 2-oxoglutarate 5-dioxygenase (PLOD1); lysosomal acid lipase (LIPA); frataxin (FXN); myostatin (MSTN); β-N-acetyl hexosaminidase A (HEXA); β-N-acetylhexosaminidase B (HEXB); β-glucocerebrosidase (GBA); adenosine monophosphate deaminase 1 (AMPD1); β-globin (HBB); iduronidase (IDUA); iduronate 2-sulfate (IDS); troponin 1 (TNN13); troponin T2 (TNNT2); troponin C (TNNC1); tropomyosin 1 (TPM1); tropomyosin 3 (TPM3); N-acetyl-α-glucosaminidase (NAGLU); N-sulfoglucosamine sulfohydrolase (SGSH); heparan-α-glucosaminide N-acetyltransferase (HGSNAT); integrin a 7 (IGTA7); integrin a 9 (IGTA9); glucosamine(N-acetyl)-6-sulfatase (GNS); galactosamine(N-acetyl)-6-sulfatase (GALNS); (3-galactosidase (GLB1); β-glucuronidase (GUSB); hyaluronoglucosaminidase 1 (HYAL1); acid ceramidase (ASAHI); galactosylcermidase (GALC); cathepsin A (CTSA); cathepsin D (CTSA); cathepsin K (CTSK); GM2 ganglioside activator (GM2A); arylsulfatase A (ARSA); arylsulfatase B (ARSB); formylglycine-generating enzyme (SUMFI); neuraminidase 1 (NEU1); N-acetylglucosamine-1-phosphate transferase a (GNPTA); N-acetylglucosamine-1-phosphate transferase β (GNPTB); N-acetylglucosamine-1-phosphate transferase γ (GNPTG); mucolipin-1 (MCOLN1); NPC intracellular transporter 1 (NPC1); NPC intracellular transporter 2 (NPC2); ceroid lipofuscinosis 5 (CLN5); ceroid lipofuscinosis 6 (CLN6); ceroid lipofuscinosis 8 (CLN8); palmitoyl protein thioesterase 1 (PPT1); tripeptidyl peptidase 1 (TPP1); battenin (CLN3); DNAJ heat shock protein family 40 member C5 (DNAJC5); major facilitator superfamily domain containing 8 (MFSD8); mannosidase a class 2B member 1 (MAN2B1); mannosidase R (MANBA); aspartylglucosaminidase (AGA); α-L-fucosidase (FUCA1); cystinosin, lysosomal cysteine transporter (CTNS); sialin; solute carrier family 2 member 10 (SLC2A10); solute carrier family 17 member 5 (SLC17A5); solute carrier family 6 member 19 (SLC6A19); solute carrier family 22 member 5 (SLC22A5); solute carrier family 37 member 4 (SLC37A4); lysosomal associated membrane protein 2 (LAMP2); sodium voltage-gated channel α subunit 4 (SCN4A); sodium voltage-gated channel R subunit 4 (SCN4B); sodium voltage-gated channel α subunit 5 (SCN5A); sodium voltage-gated channel α subunit 4 (SCN4A); calcium voltage-gated channel subunit α1c (CACNA1C); calcium voltage-gated channel subunit α1s (CACNA1S); phosphoglycerate kinase 1 (PGK1); phosphoglycerate mutase 2 (PGAM2); amylo-α-1,6-glucosidase,4-α-glucanotransferase (AGL); potassium voltage-gated channel ISK-related subfamily member 1 (KCNE1); potassium voltage-gated channel ISK-related subfamily member 2 (KCNE2); potassium voltage-gated channel subfamily J member 2 (KCNJ2); potassium voltage-gated channel subfamily J member 5 (KCNJ5); potassium voltage-gated channel subfamily H member 2 (KCNH2); potassium voltage-gated channel KQT-like subfamily member 1 (KCNQ1); hyperpolarization-activated cyclic nucleotide-gated potassium channel 4 (HCN4); chloride voltage-gated channel 1 (CLCN1); camitine palmitoyltransferase 1A (CPT1A); ryanodine receptor 1 (RYR1); ryanodine receptor 2 (RYR2); bridging integrator 1 (BIN1); LARGE xylosyl- and glucuronyltransferase 1 (LARGEl); docking protein 7 (DOK7); fukutin (FKTN); fukutin related protein (FKRP); selenoprotein N (SELENON); protein O-mannosyltransferase 1 (POMT1); protein O-mannosyltransferase 2 (POMT2); protein O-linked mannose N-acetylglucosaminyltransferase 1 (POMGNT1); protein O-linked mannose N-acetylglucosaminyltransferase 2 (POMGNT2); protein-O-mannose kinase (POMK); isoprenoid synthase domain containing (ISPD); plectin (PLEC); cholinergic receptor nicotinic epsilon subunit (CHRNE); choline O-acetyltransferase (CHAT); choline kinase β (CHKB); collagen like tail subunit of asymmetric acetylcholinesterase (COLQ); receptor associated protein of the synapse (RAPSN); four and a half LIM domains 1 (FHL1); β-1,4-glucuronyltransferase 1 (B4GAT1); β-1,3-N-acetylgalactosaminyltransferase 2 (B3GALNT2); dystroglycan 1 (DAGI); transmembrane protein 5 (TMEM5); transmembrane protein 43 (TMEM43); SECIS binding protein 2 (SECISBP2); glucosamine (UDP-N-acetyl)-2-epimerase/N-acetylmannosamine kinase (GNE); anoctamin 5 (ANO5); structural maintenance of chromosomes flexible hinge domain containing 1 (SMCHD1); lactate dehydrogenase A (LDHA); lactate dehydrogenase B (LHDB); calpain 3 (CAPN3); caveolin 3 (CAV3); tripartite motif containing 32 (TRIM32); CCHC-type zinc finger nucleic acid binding protein (CNBP); nebulin (NEB); actin, α1, skeletal muscle (ACTA1); actin, α1, cardiac muscle (ACTC1); actinin α2 (ACTN2); poly(A)-binding protein nuclear 1 (PABPN1); LEM domain-containing protein 3 (LEMD3); zinc metalloproteinase STE24 (ZMPSTE24); microsomal triglyceride transfer protein (MTTP); cholinergic receptor nicotinic α1 subunit (CHRNA1); cholinergic receptor nicotinic α2 subunit (CHRNA2); cholinergic receptor nicotinic β3 subunit (CHRNA3); cholinergic receptor nicotinic β4 subunit (CHRNA4); cholinergic receptor nicotinic β5 subunit (CHRNA5); cholinergic receptor nicotinic α6 subunit (CHRNA6); cholinergic receptor nicotinic c7 subunit (CHRNA7); cholinergic receptor nicotinic α8 subunit (CHRNA8); cholinergic receptor nicotinic α9 subunit (CHRNA9); cholinergic receptor nicotinic α10 subunit (CHRNA10); cholinergic receptor nicotinic 31 subunit (CHRNB1); cholinergic receptor nicotinic β2 subunit (CHRNB2); cholinergic receptor nicotinic β3 subunit (CHRNB3); cholinergic receptor nicotinic β4 subunit (CHRNB4); cholinergic receptor nicotinic γ subunit (CHRNG1); cholinergic receptor nicotinic a subunit (CHRND); cholinergic receptor nicotinic E subunit (CHRNE1); ATP binding cassette subfamily A member 1 (ABCA1); ATP binding cassette subfamily C member 6 (ABCC6); ATP binding cassette subfamily C member 9 (ABCC9); ATP binding cassette subfamily D member 1 (ABCD1); ATPase sarcoplasmic/endoplasmic reticulum Ca2+ transporting 1 (ATP2A1); ATM serine/threonine kinase (ATM); a tocopherol transferase protein (TTPA); kinesin family member 21A (KIF21A); paired-like homeobox 2a (PHOX2A); heparan sulfate proteoglycan 2 (HSPG2); stromal interaction molecule 1 (STIM1); notch 1 (NOTCH1); notch 3 (NOTCH3); dystrobrevin a (DTNA); protein kinase AMP-activated, noncatalytic γ2 (PRKAG2); cysteine- and glycine-rich protein 3 (CSRP3); viniculin (VCL); myozenin 2 (MyoZ2); myopalladin (MYPN); junctophilin 2 (JPH2); phospholamban (PLN); calreticulin 3 (CALR3); nexilin F-actin-binding protein (NEXN); LIM domain binding 3 (LDB3); eyes absent 4 (EYA4); huntingtin (HTT); androgen receptor (AR); protein tyrosine phosphate non-receptor type 11 (PTPN11); junction plakoglobin (JUP); desmoplakin (DSP); plakophilin 2 (PKP2); desmoglein 2 (DSG2); desmocollin 2 (DSC2); catenin α3 (CTNNA3); NK2 homeobox 5 (NKX2-5); A-kinase anchor protein 9 (AKAP9); A-kinase anchor protein 10 (AKAP10); guanine nucleotide-binding protein α-inhibiting activity polypeptide 2 (GNAI2); ankyrin 2 (ANK2); syntrophin α-1 (SNTAT); calmodulin 1 (CALM1); calmodulin 2 (CALM2); HTRA serine peptidase 1 (HTRA1); fibrillin 1 (FBN1); fibrillin 2 (FBN2); xylosyltransferase 1 (XYLT1); xylosyltransferase 2 (XYLT2); tafazzin (TAZ); homogentisate 1,2-dioxygenase (HGD); glucose-6-phosphatase catalytic subunit (G6PC); 1,4-alpha-glucan enzyme 1 (GBE1); phosphofructokinase, muscle (PFKM); phosphorylase kinase regulatory subunit alpha 1 (PHKA1); phosphorylase kinase regulatory subunit alpha 2 (PHKA2); phosphorylase kinase regulatory subunit beta (PHKB); phosphorylase kinase catalytic subunit gamma 2 (PHKG2); phosphoglycerate mutase 2 (PGAM2); cystathionine-beta-synthase (CBS); methylenetetrahydrofolate reductase (MTHFR); 5-methyltetrahydrofolate-homocysteine methyltransferase (MTR); 5-methyltetrahydrofolate-homocysteine methyltransferase reductase (MTRR); methylmalonic aciduria and homocystinuria, cblD type (MMADHC); mitochondrial DNA, including, but not limited to mitochondrially encoded NADH:ubiquinone oxidoreductase core subunit 1 (MT-ND1); mitochondrially encoded NADH:ubiquinone oxidoreductase core subunit 5 (MT-ND5); mitochondrially encoded tRNA glutamic acid (MT-TE); mitochondrially encoded tRNA histadine (MT-TH); mitochondrially encoded tRNA leucine 1 (MT-TL1); mitochondrially encoded tRNA lysine (MT-TK); mitochondrially encoded tRNA serine 1 (MT-TS1); mitochondrially encoded tRNA valine (MT-TV); mitogen-activated protein kinase 1 (MAP2K1); B-Raf proto-oncogene, serine/threonine kinase (BRAF); raf-1 proto-oncogene, serine/threonine kinase (RAF1); growth factors, including, but not limited to insulin growth factor 1 (IGF-1); transforming growth factor β3 (TGFβ3); transforming growth factor β receptor, type I (TGFβ1); transforming growth factor β receptor, type II (TGFβ2), fibroblast growth factor 2 (FGF2), fibroblast growth factor 4 (FGF4), vascular endothelial growth factor A (VEGF-A), vascular endothelial growth factor B (VEGF-B); vascular endothelial growth factor C (VEGF-C), vascular endothelial growth factor D (VEGF-D), vascular endothelial growth factor receptor 1 (VEGFR1), and vascular endothelial growth factor receptor 2 (VEGFR2); interleukins; immunoadhesins; cytokines; and antibodies.

In an aspect, a disclosed method of treating and/or preventing a genetic disease or disorder can restore the functionality and/or structural integrity of a missing, deficient, and/or mutant protein or enzyme. In an aspect, a disclosed method can comprise restoring one or more aspects of cellular homeostasis and/or cellular functionality and/or metabolic dysregulation. In an aspect, restoring one or more aspect of cellular homeostasis and/or cellular functionality and/or metabolic dysregulation comprises restoring the functionality and/or structural integrity of a missing, deficient, and/or mutant protein or enzyme.

In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a genetic disease or disorder; (vii) reversing, inhibiting, preventing, stabilizing, and/or slowing the rate of progression of a genetic disease or disorder, or (viii) any combination thereof. In an aspect, restoring one or more aspects of cellular homeostasis can comprise improving, enhancing, restoring, and/or preserving one or more aspects of cellular structural and/or functional integrity.

In an aspect, restoring the activity and/or functionality of a missing, deficient, and/or mutant protein or enzyme can comprise a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme). In an aspect, restoration can be a partial or incomplete restoration. In an aspect, restoration can be complete or near complete restoration such that the level of expression, activity, and/or functionality is similar to that of a wild-type or control level.

In an aspect of a disclosed method, techniques to monitor, measure, and/or assess the restoring one or more aspects of cellular homeostasis and/or cellular functionality can comprise qualitative (or subjective) means as well as quantitative (or objective) means. These means are known to the skilled person. For example, representative regulated variables and sensors relating to systemic homeostasis are provided below.

| Regulated Variable | Sensor |
|---|---|
| Blood Pressure/Blood | Aortic Body (Aorta), Carotid Body (Carotid |
| Volume/Na⁺ Conc./ | Artery), Atrial Volume Receptors (Heart), |
| | Juxtaglomerular Apparatus (Kidney) |
| Ca²⁺/Mg²⁺/PO₄³⁻ Conc. | Chief Cells (Parathyroid Gland) |
| Glucose | Islet of Langerhans (Pancreas) |
| Osmolarity | Circumventricular Organs (Hypothalamus) |
| pO₂, pCO₂, and pH | Aortic Body (Aorta), Carotid Body (Carotid |
| | Artery), Ventrolateral Medulla (Medulla) |
| Temperature | Thermosensory neurons (Skin), Preoptic |
| | Area (Hypothalamus) |

In an aspect of a disclosed method, contacting a cell can comprising methods known to the art. For example, contacting can comprise administering to a subject one or more disclosed compositions, disclosed isolated nucleic acid molecules, disclosed pharmaceutical formulations, and/or disclosed vectors.

In an aspect, administering can comprise intravenous, intraarterial, intramuscular, intraperitoneal, subcutaneous, intra-CSF, intrathecal, intraventricular, intrahepatic, hepatic intra-arterial, hepatic portal vein (HPV), or in utero administration. In an aspect, a disclosed composition, a disclosed isolated nucleic acid molecule, a disclosed pharmaceutical formulation, and/or a disclosed vector can be administered via intra-CSF administration in combination with RNAi, antisense oligonucleotides, miRNA, one or more small molecules, one or more therapeutic agents, one or more proteasome inhibitors, one or more immune modulators, and/or a gene editing system. In an aspect, a disclosed composition, a disclosed isolated nucleic acid molecule, a disclosed pharmaceutical formulation, and/or a disclosed vector can be administered via LNP administration. In an aspect, a disclosed composition, a disclosed isolated nucleic acid molecule, a disclosed pharmaceutical formulation, and/or a disclosed vector can be concurrently and/or serially administered to a subject via multiple routes of administration. For example, in an aspect, administering a disclosed nucleic acid molecule, a disclosed vector, and/or a disclosed pharmaceutical formulation can comprise intravenous administration and intra-cistern magna (ICM) administration. In an aspect, administering a disclosed composition, a disclosed isolated nucleic acid molecule, a disclosed pharmaceutical formulation, and/or a disclosed vector can comprise IV administration and intrathecal (ITH) administration. In an aspect, a disclosed method can employ multiple routes of administration to the subject. In an aspect, a disclosed method can employ a first route of administration that can be the same or different as a second and/or subsequent routes of administration.

In an aspect of a disclosed method of treating and/or preventing a genetic disease or disorder, a therapeutically effective amount of disclosed vector can be delivered to a subject via intravenous (IV) administration and can comprise a range of about 1×10¹⁰ vg/kg to about 2×10¹⁴ vg/kg. In an aspect, for example, a disclosed vector can be administered at a dose of about 1×10¹¹ vg/kg to about 8×10¹³ vg/kg or about 1×10¹² vg/kg to about 8×10¹³ vg/kg. In an aspect, a disclosed vector can be administered at a dose of about 1×10¹³ vg/kg to about 6×10¹³ vg/kg. In an aspect, a disclosed vector can be administered at a dose of at least about 1×10¹⁰ vg/kg, at least about 5×10¹⁰ vg/kg, at least about 1×10¹¹ vg/kg, at least about 5×10¹¹ vg/kg, at least about 1×10¹² vg/kg, at least about 5×10¹² vg/kg, at least about 1×10¹³ vg/kg, at least about 5×10¹³ vg/kg, or at least about 1×10¹⁴ vg/kg. In an aspect, a disclosed vector can be administered at a dose of no more than about 1×10¹⁰ vg/kg, no more than about 5×10¹⁰ vg/kg, no more than about 1×10¹¹ vg/kg, no more than about 5×10¹¹ vg/kg, no more than about 1×10¹² vg/kg, no more than about 5×10¹² vg/kg, no more than about 1×10¹³ vg/kg, no more than about 5×10¹³, or no more than about 1×10¹⁴ vg/kg. In an aspect, a disclosed vector can be administered to a subject at a dose of about 1×10¹² vg/kg. In an aspect, a disclosed vector can be administered to a subject at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results.

In an aspect, a disclosed method of treating and/or preventing a genetic disease or disorder can further comprise administering to the subject a therapeutically effective amount of a therapeutic agent. A therapeutic agent can be any disclosed agent that effects a desired clinical outcome.

In an aspect, a disclosed method of treating and/or preventing a genetic disease or disorder can further comprise monitoring the subject for adverse effects. In an aspect, in the absence of adverse effects, the method can further comprise continuing to treat the subject. In an aspect, in the presence of adverse effects, the method can further comprise modifying the treating step. Methods of monitoring a subject's well-being can include both subjective and objective criteria (and are discussed supra). Such methods are known to the skilled person.

In an aspect, a disclosed method can further comprise administering to the subject a therapeutically effective amount of an agent that can correct one or more aspects of a dysregulated metabolic or enzymatic pathway. In an aspect, such an agent can comprise an enzyme for enzyme replacement therapy. In an aspect, a disclosed enzyme can replace any enzyme in a dysregulated or dysfunctional metabolic or enzymatic pathway. In an aspect, a disclosed method can comprise replacing one or more enzymes in a dysregulated or dysfunctional metabolic pathway.

In an aspect, a disclosed method of treating and/or preventing a genetic disease or disorder can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus. In an aspect, a disclosed immune modulator such as methotrexate can be administered at a transient low to high dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with a disclosed therapeutic agent. In an aspect, a disclosed immune modulator can be administered orally about one hour or a few days before a disclosed isolated nucleic acid molecule, a disclosed vector, a disclosed pharmaceutical formulation, or a combination thereof. In an aspect, a disclosed immune modulator can be administered subcutaneously about 15 minutes before or a few days before a disclosed isolated nucleic acid molecule, a disclosed vector, a disclosed pharmaceutical formulation, or a combination thereof. In an aspect, a disclosed immune modulator can be administered concurrently with a disclosed isolated nucleic acid molecule, a disclosed vector, a disclosed pharmaceutical formulation, or a combination thereof.

In an aspect, a disclosed method of treating and/or preventing a genetic disease or disorder can further comprise administering one or more proteasome inhibitors (e.g., bortezomib, carfilzomib, marizomib, ixazomib, and oprozomib). In an aspect, a proteasome inhibitor can be an agent that acts on plasma cells (e.g., daratumumab). In an aspect, an agent that acts on a plasma cell can be melphalan hydrochloride, melphalan, pamidronate disodium, carmustine, carfilzomib, carmustine, cyclophosphamide, daratumumab, doxorubicin hydrochloride liposome, doxorubicin hydrochloride liposome, elotuzumab, melphalan hydrochloride, panobinostat, ixazomib citrate, carfilzomib, lenalidomide, melphalan, melphalan hydrochloride, plerixafor, ixazomib citrate, pamidronate disodium, panobinostat, plerixafor, pomalidomide, pomalidomide, lenalidomide, selinexor, thalidomide, thalidomide, bortezomib, selinexor, zoledronic acid, or zoledronic acid.

In an aspect, a disclosed method of improving transgene stability can further comprise administering one or more proteasome inhibitors or agents that act on plasma cells prior to administering a disclosed isolated nucleic acid molecule, a disclosed vector, or a disclosed pharmaceutical formulation. In an aspect, a disclosed method can comprise administering one or more proteasome inhibitors or one or more agents that act on plasma cells concurrently with administering a disclosed isolated nucleic acid molecule, a disclosed vector, or a disclosed pharmaceutical formulation. In an aspect, a disclosed method can comprise administering one or more proteasome inhibitors or one or more agents that act on plasma cells subsequent to administering a disclosed isolated nucleic acid molecule, a disclosed vector, or a disclosed pharmaceutical formulation. In an aspect, a disclosed method can further comprise administering one or more proteasome inhibitors more than 1 time. In an aspect, a disclosed method can comprise administering one or more proteasome inhibitors repeatedly over time.

In an aspect, a disclosed method of treating and/or preventing a genetic disease or disorder can further comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time. In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed method of treating and/or preventing a genetic disease or disorder can further comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect, a disclosed method can further comprise administering lipid nanoparticles (LNPs). In an aspect, LNPs can be organ-targeted. In an aspect, LNPs can be liver-targeted or testes-targeted. For example, in an aspect, mRNA therapy with LNP encapsulation for systemic delivery to a subject has the potential to restore the functionality and/or structural integrity of a missing, deficient, and/or mutant protein or enzyme.

In an aspect, a disclosed method of treating and/or preventing a genetic disease or disorder can further comprise treating a subject that has developed or is likely to develop neutralizing antibodies (ABs) to a disclosed vector, a disclosed capsid, and/or a disclosed transgene. In an aspect, treating a subject that has developed or is likely to develop neutralizing antibodies can comprise plasmapheresis and immunosuppression. In an aspect, a disclosed method can comprise using immunosuppression to decrease the T cell, B cell, and/or plasma cell population, decrease the innate immune response, inflammatory response, and antibody levels in general. In an aspect, a disclosed method can comprise administering an IgG-degrading agent that depletes pre-existing neutralizing antibodies. In an aspect, a disclosed method can comprise administering to the subject IdeS or IdeZ, rapamycin, and/or SVP-Rapamycin. In an aspect, a disclosed method can comprise administering Tacrolimus. In an aspect, a disclosed IgG-degrading agent is bacteria-derived IdeS or IdeZ.

In an aspect, a disclosed method can comprise repeating a disclosed administering step such as, for example, repeating the administering of a disclosed isolated nucleic acid molecule, a disclosed vector, a disclosed pharmaceutical formulation, a disclosed therapeutic agent, a disclosed immune modulator, a disclosed proteasome inhibitor, a disclosed immunosuppressive agent, a disclosed compound that exerts a therapeutic effect against B cells and/or a disclosed compound that targets or alters antigen presentation or humoral or cell mediated immune response.

In an aspect, a disclosed method can comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, or by changing the duration of time one or more of the disclosed isolated nucleic acid molecules, disclosed vectors, disclosed pharmaceutical formulations, or a combination are administered to a subject.

In an aspect, a method can be altered by changing the amount of one or more disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject, or by changing the frequency of administration of one or more of the disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject.

In as aspect, a disclosed method can comprise concurrent administration of one or more of the following: one or more disclosed isolated nucleic acid molecules, one or more disclosed vectors, one or more disclosed pharmaceutical formulations, one or more disclosed therapeutic agents, one or more disclosed immune modulators, one or more disclosed proteasome inhibitors, one or more disclosed immunosuppressive agents, one or more disclosed compounds that exert therapeutic effect against B cells, one or more disclosed compounds that targets or alters antigen presentation or humoral or cell mediated immune response, or any combination thereof.

In an aspect, a disclosed immune modulator can be administered prior to or after the administration of a disclosed therapeutic agent.

In an aspect, a disclosed method of treating and/or preventing a genetic disease or disorder can further comprise generating a disclosed isolated nucleic acid molecule. In an aspect, a disclosed method can further comprise generating a disclosed viral or non-viral vector. In an aspect, generating a disclosed viral vector can comprise generating an AAV vector or a recombinant AAV (such as those disclosed herein). In an aspect, a disclosed method can further comprise gene editing one or more relevant genes (such as, for example, a missing, deficient, and/or mutant protein or enzyme), wherein editing includes but is not limited to single gene knockout, loss of function screening of multiple genes at one, gene knockin, or a combination thereof.

In an aspect, a disclosed method of treating and/or preventing a genetic disease or disorder can further comprise administering an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable. In an aspect, a disclosed oligonucleotide therapeutic agent can comprise a CRISPR-based endonuclease. In an aspect, a disclosed endonuclease can be Cas9. In an aspect, a disclosed Cas9 can be from Staphylococcus aureus or Streptococcus pyogenes. Cas9 can have the amino acid sequence set forth in SEQ ID NO:32, SEQ ID NO:33, or a fragment thereof. In an aspect, a disclosed Cas9 can have a sequence having at least 75%, at least 80%, at least 85%, at least 90%, or at least 95% identity to the amino acid sequence set forth in SEQ ID NO:32, SEQ ID NO:33, or a fragment thereof. In an aspect, a disclosed nucleic acid sequence for Cas9 can comprise the sequence set forth in SEQ ID NO:31 or a fragment thereof. In an aspect, a disclosed nucleic acid sequence for Cas9 can comprise a sequence having at least 80%, at least 85%, at least 90%, or at least 95% identity to the sequence set forth in SEQ ID NO:31 or a fragment thereof. In an aspect, a disclosed method can comprise administering the subject a disclosed RNA therapeutic.

In an aspect, a disclosed method of treating and/or preventing a genetic disease or disorder can further comprise generating and/or validating one or more of the disclosed isolated nucleic acid molecules, one or more of the disclosed vectors, one or more of the disclosed pharmaceutical formulations, or any combination thereof.

### E. Kits

Disclosed herein is a kit comprising a disclosed isolated nucleic acid molecule, a disclosed vector, a disclosed pharmaceutical formulation, or any combination thereof. Disclosed herein is a kit comprising one or more disclosed isolated nucleic acid molecules, one or more disclosed vectors, one or more disclosed pharmaceutical formulations, or any combination thereof. In an aspect, a kit can comprise a disclosed isolated nucleic acid molecule, a disclosed vector, a disclosed pharmaceutical formulation, a disclosed RNA therapeutic, or a combination thereof, and one or more agents. “Agents” and “Therapeutic Agents” are known to the art and are described supra.

In an aspect, the one or more agents can treat, prevent, inhibit, and/or ameliorate one or more comorbidities in a subject. In an aspect, one or more active agents can treat, inhibit, prevent, and/or ameliorate cellular and/or metabolic complications related to a missing, deficient, and/or mutant protein or enzyme.

In an aspect, a disclosed kit can comprise at least two components constituting the kit. Together, the components constitute a functional unit for a given purpose (such as, for example, treating a subject diagnosed with or suspected of having a disease or disorder). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed isolated nucleic acid molecule, a disclosed vector, a disclosed pharmaceutical formulation, a disclosed RNA therapeutic, or a combination thereof, and a label or package insert with instructions for use. In an aspect, suitable containers include, for example, bottles, vials, syringes, blister pack, etc. The containers can be formed from a variety of materials such as glass or plastic. The container can hold a disclosed isolated nucleic acid molecule, a disclosed vector, a disclosed pharmaceutical formulation, or a combination thereof, and can have a sterile access port (for example the container may be an intravenous solution bag or a vial having a stopper pierceable by a hypodermic injection needle). The label or package insert can indicate that a disclosed isolated nucleic acid molecule, a disclosed vector, a disclosed pharmaceutical formulation, a disclosed RNA therapeutic, or a combination thereof can be used for treating, preventing, inhibiting, and/or ameliorating a disease or disorder or complications and/or symptoms associated with a disease or disorder. A kit can comprise additional components necessary for administration such as, for example, other buffers, diluents, filters, needles, and syringes.

In an aspect, a disclosed kit can be used to generate one or more chimeric RNA molecules. In an aspect, a disclosed kit can be used to treat and/or prevent a disease or disorder.

### F. Miscellaneous

Disclosed herein is a nucleic acid molecule comprising (i) one or more guide RNA sequences that target binding of the nucleic acid molecule to the intron immediately 3′ to the last exon of the endogenous transcript that will be included in the chimeric trans-spliced RNA; (ii) a 3′ splice region comprising a branch point (nucleotide sequence: YNYYRAY, where Y is a pyrimidine and R is a purine (SEQ ID NO:57)), a pyrimidine tract and a 3′ splice acceptor site (nucleotide sequence: YAG, where Y is a pyrimidine (SEQ ID NO:58)); (iii) a spacer region that separates the 3′ splice region from the guide RNA; (iv) a nucleotide sequence to be trans-spliced to the target pre-mRNA; wherein said nucleic acid molecule is recognized by nuclear splicing components within the cell; (v) one or more stem loops for interaction with the RNA binding protein; and (vi) a nucleotide sequence encoding an RNA binding protein capable of binding the stem loop structure and that stabilizes the guide sequence.

Disclosed herein is a nucleic acid molecule comprising (i) one or more guide RNA sequences that target binding of the nucleic acid molecule to the intron immediately 5′ to the first exon of the endogenous transcript that will be included in the chimeric trans-spliced RNA; (ii) a 5′ splice donor site (nucleotide sequence: GT (SEQ ID NO:59)); (iii) a spacer region that separates the 5′ splice region from the guide RNA; (iv) a nucleotide sequence to be trans-spliced to the target pre-mRNA; wherein said nucleic acid molecule is recognized by nuclear splicing components within the cell; (vi) one or more stem loops for interaction with the RNA binding protein; and (vii) a nucleotide sequence encoding an RNA binding protein capable of binding the stem loop structure and that stabilizes the guide sequence.

Disclosed herein is a nucleic acid molecule comprising (i) two or more guide RNA sequences that target binding of the nucleic acid molecule to the intron immediately 5′ and 3′ to the target exon of the endogenous transcript that will be replaced via trans-splicing in the chimeric RNA molecule; (ii) a 3′ splice region comprising a branch point (nucleotide sequence: YNYYRAY, where Y is a pyrimidine and R is a purine(SEQ ID NO:57)), a pyrimidine tract and a 3′ splice acceptor site (nucleotide sequence: YAG, where Y is a pyrimidine (SEQ ID NO:58)); (iii) a 5′ splice donor site (nucleotide sequence: GT (SEQ ID NO:59)); (iv) a spacer region that separates the 3′ splice region from the guide sequence; (iv) a nucleotide sequence to be trans-spliced to the target pre-mRNA; wherein said nucleic acid molecule is recognized by nuclear splicing components within the cell; (v) two or more stem loops for interaction with the RNA binding protein; and (vi) a nucleotide sequence encoding an RNA binding protein capable of binding the stem loop structure and that stabilizes the guide sequence.

Disclosed herein is a vector comprising a nucleic acid molecule comprising (i) one or more guide RNA sequences that target binding of the nucleic acid molecule to the intron immediately 3′ to the last exon of the endogenous transcript that will be included in the chimeric trans-spliced RNA; (ii) a 3′ splice region comprising a branch point (nucleotide sequence: YNYYRAY, where Y is a pyrimidine and R is a purine (SEQ ID NO:57)), a pyrimidine tract and a 3′ splice acceptor site (nucleotide sequence: YAG, where Y is a pyrimidine (SEQ ID NO:58)); (iii) a spacer region that separates the 3′ splice region from the guide RNA; (iv) a nucleotide sequence to be trans-spliced to the target pre-mRNA; wherein said nucleic acid molecule is recognized by nuclear splicing components within the cell; (v) one or more stem loops for interaction with the RNA binding protein; and (vi) a nucleotide sequence encoding an RNA binding protein capable of binding the stem loop structure and that stabilizes the guide sequence.

Disclosed herein is a vector comprising a nucleic acid molecule comprising (i) one or more guide RNA sequences that target binding of the nucleic acid molecule to the intron immediately 5′ to the first exon of the endogenous transcript that will be included in the chimeric trans-spliced RNA; (ii) a 5′ splice donor site (nucleotide sequence: GT (SEQ ID NO:59)); (iii) a spacer region that separates the 5′ splice region from the guide RNA; (iv) a nucleotide sequence to be trans-spliced to the target pre-mRNA; wherein said nucleic acid molecule is recognized by nuclear splicing components within the cell; (vi) one or more stem loops for interaction with the RNA binding protein; and (vii) a nucleotide sequence encoding an RNA binding protein capable of binding the stem loop structure and that stabilizes the guide sequence.

Disclosed herein is a vector comprising a nucleic acid molecule comprising (i) two or more guide RNA sequences that target binding of the nucleic acid molecule to the intron immediately 5′ and 3′ to the target exon of the endogenous transcript that will be replaced via trans-splicing in the chimeric RNA molecule; (ii) a 3′ splice region comprising a branch point (nucleotide sequence: YNYYRAY, where Y is a pyrimidine and R is a purine (SEQ ID NO:58)), a pyrimidine tract and a 3′ splice acceptor site (nucleotide sequence: YAG, where Y is a pyrimidine (SEQ ID NO:59)); (iii) a 5′ splice donor site (nucleotide sequence: GT (SEQ ID NO:59)); (iv) a spacer region that separates the 3′ splice region from the guide sequence; (iv) a nucleotide sequence to be trans-spliced to the target pre-mRNA; wherein said nucleic acid molecule is recognized by nuclear splicing components within the cell; (v) two or more stem loops for interaction with the RNA binding protein; and (vi) a nucleotide sequence encoding an RNA binding protein capable of binding the stem loop structure and that stabilizes the guide sequence.

Disclosed is a cell comprising a nucleic acid molecule comprising (i) one or more guide RNA sequences that target binding of the nucleic acid molecule to the intron immediately 3′ to the last exon of the endogenous transcript that will be included in the chimeric trans-spliced RNA; (ii) a 3′ splice region comprising a branch point (nucleotide sequence: YNYYRAY, where Y is a pyrimidine and R is a purine (SEQ ID NO:57)), a pyrimidine tract and a 3′ splice acceptor site (nucleotide sequence: YAG, where Y is a pyrimidine (SEQ ID NO:58)); (iii) a spacer region that separates the 3′ splice region from the guide RNA; (iv) a nucleotide sequence to be trans-spliced to the target pre-mRNA; wherein said nucleic acid molecule is recognized by nuclear splicing components within the cell; (v) one or more stem loops for interaction with the RNA binding protein; and (vi) a nucleotide sequence encoding an RNA binding protein capable of binding the stem loop structure and that stabilizes the guide sequence.

Disclosed is a cell comprising a nucleic acid molecule comprising (i) one or more guide RNA sequences that target binding of the nucleic acid molecule to the intron immediately 5′ to the first exon of the endogenous transcript that will be included in the chimeric trans-spliced RNA; (ii) a 5′ splice donor site (nucleotide sequence: GT (SEQ ID NO:59)); (iii) a spacer region that separates the 5′ splice region from the guide RNA; (iv) a nucleotide sequence to be trans-spliced to the target pre-mRNA; wherein said nucleic acid molecule is recognized by nuclear splicing components within the cell; (vi) one or more stem loops for interaction with the RNA binding protein; and (vii) a nucleotide sequence encoding an RNA binding protein capable of binding the stem loop structure and that stabilizes the guide sequence.

Disclosed is a cell comprising a nucleic acid molecule comprising (i) two or more guide RNA sequences that target binding of the nucleic acid molecule to the intron immediately 5′ and 3′ to the target exon of the endogenous transcript that will be replaced via trans-splicing in the chimeric RNA molecule; (ii) a 3′ splice region comprising a branch point (nucleotide sequence: YNYYRAY, where Y is a pyrimidine and R is a purine (SEQ ID NO:57)), a pyrimidine tract and a 3′ splice acceptor site (nucleotide sequence: YAG, where Y is a pyrimidine (SEQ ID NO:58)); (iii) a 5′ splice donor site (nucleotide sequence: GT (SEQ ID NO:59)); (iv) a spacer region that separates the 3′ splice region from the guide sequence; (iv) a nucleotide sequence to be trans-spliced to the target pre-mRNA; wherein said nucleic acid molecule is recognized by nuclear splicing components within the cell; (v) two or more stem loops for interaction with the RNA binding protein; and (vi) a nucleotide sequence encoding an RNA binding protein capable of binding the stem loop structure and that stabilizes the guide sequence.

Disclosed herein is a composition for transcriptome editing in a mammalian cells comprising an adeno-associated virus (AAV) vector and a nucleic acid molecule comprising (i) one or more guide RNA sequences that target binding of the nucleic acid molecule to the intron immediately 3′ to the last exon of the endogenous transcript that will be included in the chimeric trans-spliced RNA; (ii) a 3′ splice region comprising a branch point (nucleotide sequence: YNYYRAY, where Y is a pyrimidine and R is a purine (SEQ ID NO:57)), a pyrimidine tract and a 3′ splice acceptor site (nucleotide sequence: YAG, where Y is a pyrimidine (SEQ ID NO:58)); (iii) a spacer region that separates the 3′ splice region from the guide RNA; (iv) a nucleotide sequence to be trans-spliced to the target pre-mRNA; wherein said nucleic acid molecule is recognized by nuclear splicing components within the cell; (v) one or more stem loops for interaction with the RNA binding protein; and (vi) a nucleotide sequence encoding an RNA binding protein capable of binding the stem loop structure and that stabilizes the guide sequence.

Disclosed herein is a composition for transcriptome editing in a mammalian cells comprising an adeno-associated virus (AAV) vector and a nucleic acid molecule comprising (i) one or more guide RNA sequences that target binding of the nucleic acid molecule to the intron immediately 5′ to the first exon of the endogenous transcript that will be included in the chimeric trans-spliced RNA; (ii) a 5′ splice donor site (nucleotide sequence: GT ((SEQ ID NO:59); (iii) a spacer region that separates the 5′ splice region from the guide RNA; (iv) a nucleotide sequence to be trans-spliced to the target pre-mRNA; wherein said nucleic acid molecule is recognized by nuclear splicing components within the cell; (vi) one or more stem loops for interaction with the RNA binding protein; and (vii) a nucleotide sequence encoding an RNA binding protein capable of binding the stem loop structure and that stabilizes the guide sequence.

Disclosed herein is a composition for transcriptome editing in a mammalian cells comprising an adeno-associated virus (AAV) vector and a nucleic acid molecule comprising (i) two or more guide RNA sequences that target binding of the nucleic acid molecule to the intron immediately 5′ and 3′ to the target exon of the endogenous transcript that will be replaced via trans-splicing in the chimeric RNA molecule; (ii) a 3′ splice region comprising a branch point (nucleotide sequence: YNYYRAY, where Y is a pyrimidine and R is a purine (SEQ ID NO:57)), a pyrimidine tract and a 3′ splice acceptor site (nucleotide sequence: YAG, where Y is a pyrimidine (SEQ ID NO:58)); (iii) a 5′ splice donor site (nucleotide sequence: GT (SEQ ID NO:59)); (iv) a spacer region that separates the 3′ splice region from the guide sequence; (iv) a nucleotide sequence to be trans-spliced to the target pre-mRNA; wherein said nucleic acid molecule is recognized by nuclear splicing components within the cell; (v) two or more stem loops for interaction with the RNA binding protein; and (vi) a nucleotide sequence encoding an RNA binding protein capable of binding the stem loop structure and that stabilizes the guide sequence.

Disclosed herein is a method of producing a chimeric RNA molecule in a cell comprising contacting a target pre-mRNA expressed in the cell via RNP mediated ternary complex with a nucleic acid molecule comprising a nucleic acid molecule comprising (i) one or more guide RNA sequences that target binding of the nucleic acid molecule to the intron immediately 3′ to the last exon of the endogenous transcript that will be included in the chimeric trans-spliced RNA; (ii) a 3′ splice region comprising a branch point (nucleotide sequence: YNYYRAY, where Y is a pyrimidine and R is a purine (SEQ ID NO:57)), a pyrimidine tract and a 3′ splice acceptor site (nucleotide sequence: YAG, where Y is a pyrimidine (SEQ ID NO:58)); (iii) a spacer region that separates the 3′ splice region from the guide RNA; (iv) a nucleotide sequence to be trans-spliced to the target pre-mRNA; wherein said nucleic acid molecule is recognized by nuclear splicing components within the cell; (v) one or more stem loops for interaction with the RNA binding protein; and (vi) a nucleotide sequence encoding an RNA binding protein capable of binding the stem loop structure and that stabilizes the guide sequence, wherein the nucleic acid molecule is recognized by nuclear splicing components.

Disclosed herein is a method of producing a chimeric RNA molecule in a cell comprising contacting a target pre-mRNA expressed in the cell via RNP mediated ternary complex with a nucleic acid molecule comprising a nucleic acid molecule comprising (i) one or more guide RNA sequences that target binding of the nucleic acid molecule to the intron immediately 5′ to the first exon of the endogenous transcript that will be included in the chimeric trans-spliced RNA; (ii) a 5′ splice donor site (nucleotide sequence: GT (SEQ ID NO:59)); (iii) a spacer region that separates the 5′ splice region from the guide RNA; (iv) a nucleotide sequence to be trans-spliced to the target pre-mRNA; wherein said nucleic acid molecule is recognized by nuclear splicing components within the cell; (vi) one or more stem loops for interaction with the RNA binding protein; and (vii) a nucleotide sequence encoding an RNA binding protein capable of binding the stem loop structure and that stabilizes the guide sequence, wherein the nucleic acid molecule is recognized by nuclear splicing components.

Disclosed herein is a method of producing a chimeric RNA molecule in a cell comprising contacting a target pre-mRNA expressed in the cell via RNP mediated ternary complex with a nucleic acid molecule comprising a nucleic acid molecule comprising (i) two or more guide RNA sequences that target binding of the nucleic acid molecule to the intron immediately 5′ and 3′ to the target exon of the endogenous transcript that will be replaced via trans-splicing in the chimeric RNA molecule; (ii) a 3′ splice region comprising a branch point (nucleotide sequence: YNYYRAY, where Y is a pyrimidine and R is a purine (SEQ ID NO:57)), a pyrimidine tract and a 3′ splice acceptor site (nucleotide sequence: YAG, where Y is a pyrimidine (SEQ ID NO:58)); (iii) a 5′ splice donor site (nucleotide sequence: GT (SEQ ID NO:59)); (iv) a spacer region that separates the 3′ splice region from the guide sequence; (iv) a nucleotide sequence to be trans-spliced to the target pre-mRNA; wherein said nucleic acid molecule is recognized by nuclear splicing components within the cell; (v) two or more stem loops for interaction with the RNA binding protein; and (vi) a nucleotide sequence encoding an RNA binding protein capable of binding the stem loop structure and that stabilizes the guide sequence, wherein the nucleic acid molecule is recognized by nuclear splicing components.

In an aspect, the chimeric RNA molecule can comprise a sequence encoding a translatable protein. In an aspect, a disclosed nucleic acid sequence to be trans-spliced to the target RNA can comprise a sequence encoding the DP71 protein or the DMD protein. In an aspect, a disclosed chimeric RNA molecule can comprise a sequence encoding the DP71 protein or DMD protein. In an aspect, a disclosed vector can comprise a disclosed nucleic acid comprising a sequence encoding the DP71 protein or the DMD protein. In an aspect, a disclosed vector can comprise a chimeric RNA molecule comprising a sequence encoding the DP71 or DMD protein. In an aspect, a disclosed cell can comprise a disclosed nucleic acid comprising a sequence encoding the DP71 protein or the DMD protein. In an aspect, a disclosed cell can comprise a chimeric RNA molecule comprising a sequence encoding the DP71 or DMD protein.

In an aspect, a disclosed nucleic acid sequence to be trans-spliced to the target RNA can comprise a sequence encoding the DMPK protein. In an aspect, a disclosed chimeric RNA molecule can comprise a sequence encoding the DMPK protein. In an aspect, a disclosed vector can comprise a disclosed nucleic acid comprising a sequence encoding the DMPK protein. In an aspect, a disclosed vector can comprise a chimeric RNA molecule comprising a sequence encoding the DMPK protein. In an aspect, a disclosed cell can comprise a disclosed nucleic acid comprising a sequence encoding the DMPK protein. In an aspect, a disclosed cell can comprise a chimeric RNA molecule comprising a sequence encoding the DMPK.

In an aspect, a disclosed nucleic acid sequence to be trans-spliced to the target RNA can comprise a sequence encoding the LMNA protein. In an aspect, a disclosed chimeric RNA molecule can comprise a sequence encoding the LMNA protein. In an aspect, a disclosed vector can comprise a disclosed nucleic acid comprising a sequence encoding the LMNA protein. In an aspect, a disclosed vector can comprise a chimeric RNA molecule comprising a sequence encoding the LMNA protein. In an aspect, a disclosed cell can comprise a disclosed nucleic acid comprising a sequence encoding the LMNA protein. In an aspect, a disclosed cell can comprise a chimeric RNA molecule comprising a sequence encoding the LMNA protein.

In an aspect, a disclosed nucleic acid sequence to be trans-spliced to the target RNA can comprise a sequence encoding the LRRK2 protein. In an aspect, a disclosed chimeric RNA molecule can comprise a sequence encoding the LRRK2 protein. In an aspect, a disclosed vector can comprise a disclosed nucleic acid comprising a sequence encoding the LRRK2 protein. In an aspect, a disclosed vector can comprise a chimeric RNA molecule comprising a sequence encoding the LRRK2 protein. In an aspect, a disclosed cell can comprise a disclosed nucleic acid comprising a sequence encoding the LRRK2 protein. In an aspect, a disclosed cell can comprise a chimeric RNA molecule comprising a sequence encoding the LRRK2 protein.

In an aspect, a disclosed nucleic acid sequence to be trans-spliced to the target RNA can comprise a sequence encoding the CFTR protein. In an aspect, a disclosed chimeric RNA molecule can comprise a sequence encoding the CFTR protein. In an aspect, a disclosed vector can comprise a disclosed nucleic acid comprising a sequence encoding the CFTR protein. In an aspect, a disclosed vector can comprise a chimeric RNA molecule comprising a sequence encoding the CFTR protein. In an aspect, a disclosed cell can comprise a disclosed nucleic acid comprising a sequence encoding the CFTR protein. In an aspect, a disclosed cell can comprise a chimeric RNA molecule comprising a sequence encoding the CFTR protein.

In an aspect, a disclosed RNA binding protein can comprise a Type VI CRISPR enzyme.

### VIII. EXAMPLES

As there are pathogenic mutations in more than 500 genes exceeding the packaging capacity of AAV, several efforts have been aimed at circumventing this barrier to expression of large genes. While these techniques differ, the general approach remains broadly similar between strategies. Briefly, a dual AAV vector approach is taken; wherein the DNA sequence encoding the protein of interest is split and packaged into separate vectors. Upon co-infection of target cells by the two vectors, the genomes of the two vectors recombine with each other via inverted repeat sequences or overlapping complementary sequences forming a single genome bearing the reconstituted DNA sequence expressing the full protein of interest. While this strategy is feasible the efficiency of recombination between genomes has limited the viability of its widespread adoption.

Separately, other efforts to effect phenotypic correction of genes ineligible for classical AAV mediated gene therapy have inspired an approach involving the manipulation of endogenous messenger RNA, the conduit between DNA and protein. The RNA editing strategy known as spliceosome mediated RNA trans-splicing (SMART) has been developed as a strategy to introduce large precise modifications to the primary structure of RNA transcripts independent of target transcript length. The aim of this approach is to hijack the cellular RNA processing machinery for incorporation of a desired sequence into an endogenous transcript. In this strategy, a recombinant RNA molecule is introduced to the cell comprising 3 essential components: an RNA targeting motif, a hemi intron sequence, and the primary sequence of the desired RNA to be joined to an endogenous RNA transcript. The RNA targeting motif is comprised of a stretch of oligonucleotides anti-sense to an intron of an endogenous target pre-mRNA. Upon Watson-Crick base pairing of the RNA targeting motif with the endogenous intron, the hemi intron is then recognized by the spliceosome. Depending on the desired splicing reaction either a 5′ hemi intron facilitates the splicing of the trans-splicing molecule to the exon immediately 3′ to the targeted intron, or a 3′ hemi intron facilitates the splicing of the trans-splicing molecule to the exon immediately 5′ to the targeted intron. This produces a mature chimeric RNA transcript comprised of the recombinant 5′ start of a transcript joined to an endogenous 3′ sequence or endogenous 5′ start of a transcript joined to a recombinant 3′ sequence, respectively. The utility of this strategy is that a single AAV vector needs only to package a genome capable of producing a trans-splicing RNA molecule containing the sequence for part of a gene, obviating the need to deliver the full-length protein coding sequence to a cell. As the entire recombinant RNA region of a chimeric RNA product may be specified by the user, the trans-splicing RNA may contain the wild type sequence of a target RNA, an inactivating mutation in the target RNA, or a modified RNA sequence encoding a novel protein. However, similar to the split-AAV vector approach, the low specificity and efficiency of RNA targeting by anti-sense RNA sequences has precluded the widespread use of this technology in research and clinical settings.

Disclosed herein is a Clustered Regularly Interspaced Short Palindromic Repeat (CRISPR)/CRISPR-associated (Cas) 13-based system for transcriptome engineering. The compositions and methods disclosed herein are directed to a Cas13 mediated approach for splicing in trans of recombinant RNA sequences to endogenous target pre-RNA messages for the production of chimeric RNA transcripts.

### Example 1

### Validation of 3′ Trans-Splicing in the DP71 Transcript

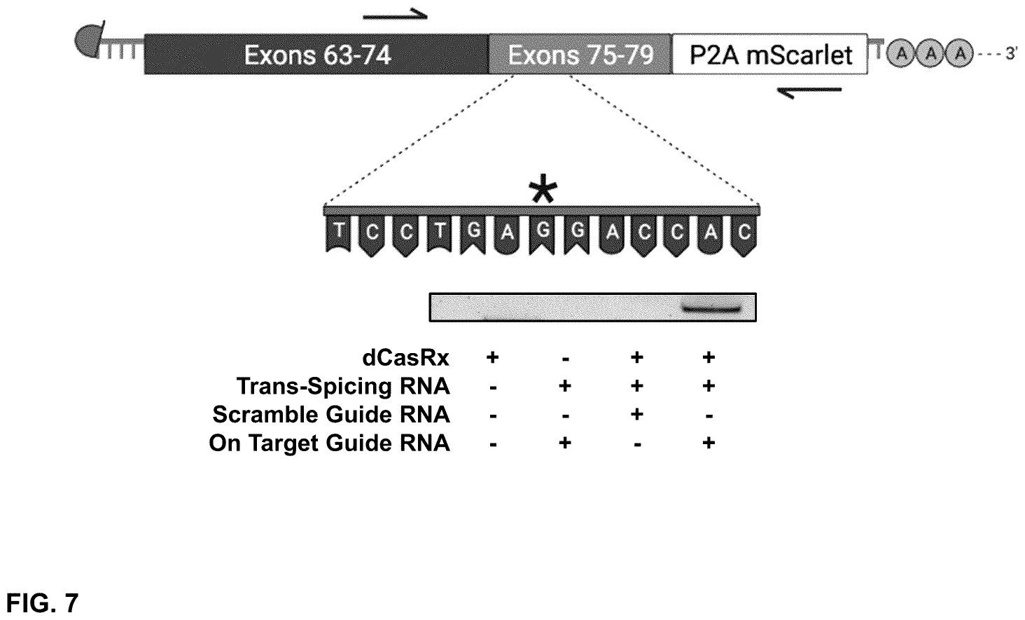

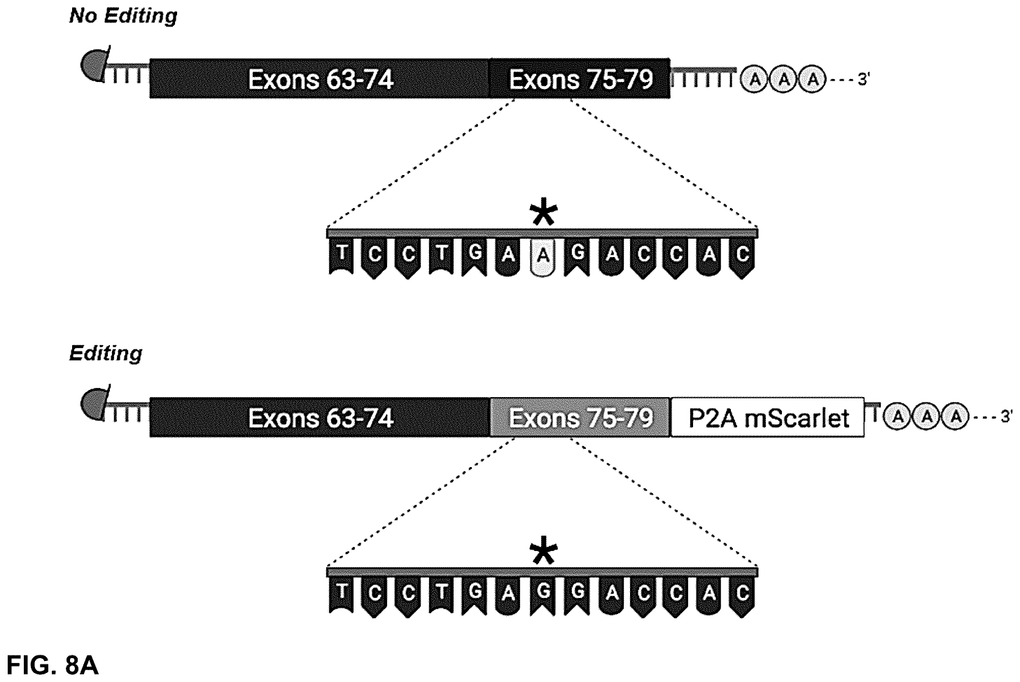

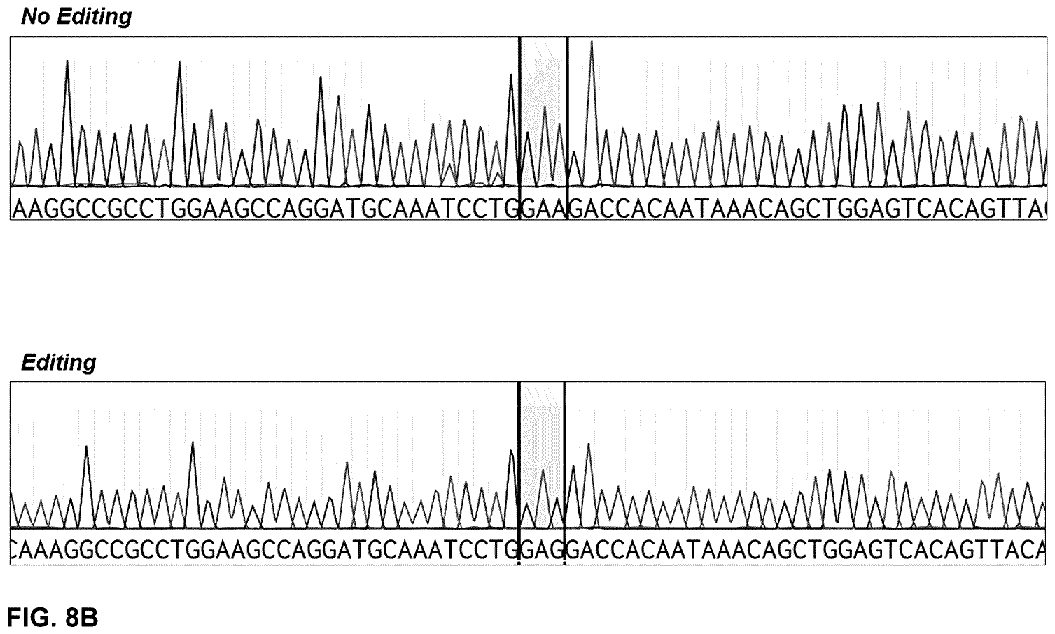

A PCR based validation of 3′ editing at the DMD locus in HEK293 cells was developed. FIG. 7 (top) depicts what a trans-spliced DP71 transcript would look like comprised of the endogenous 5′ exons of the transcript (black) the trans-spliced remaining exons (gray) and an additional mScarlett tag on the trans-spliced exons. RT-PCR across the splice junction was used as a primary endpoint of trans-splicing validation. Cells transfected with either the dCasRx (lane 1) or trans-splicing RNA (lane 2) expression plasmid did not yield detectable trans-splicing. Further, when the dCasRx expression plasmid is transfected with a trans-splicing RNA expression plasmid lacking a targeting sequence to the transcript of interest (lane 3), trans-splicing was not detected. But, when dCasRx expression plasmid was transfected with the trans-splicing expression plasmid containing an on-target guide sequence, trans-splicing was detected by RT-PCR (lane 4). This band was confirmed to be the trans-spliced RNA by sanger sequencing of the band to detect a single nucleotide polymorphism (A>G [E3580]) encoded uniquely in the trans-spliced RNA product (FIG. 8).

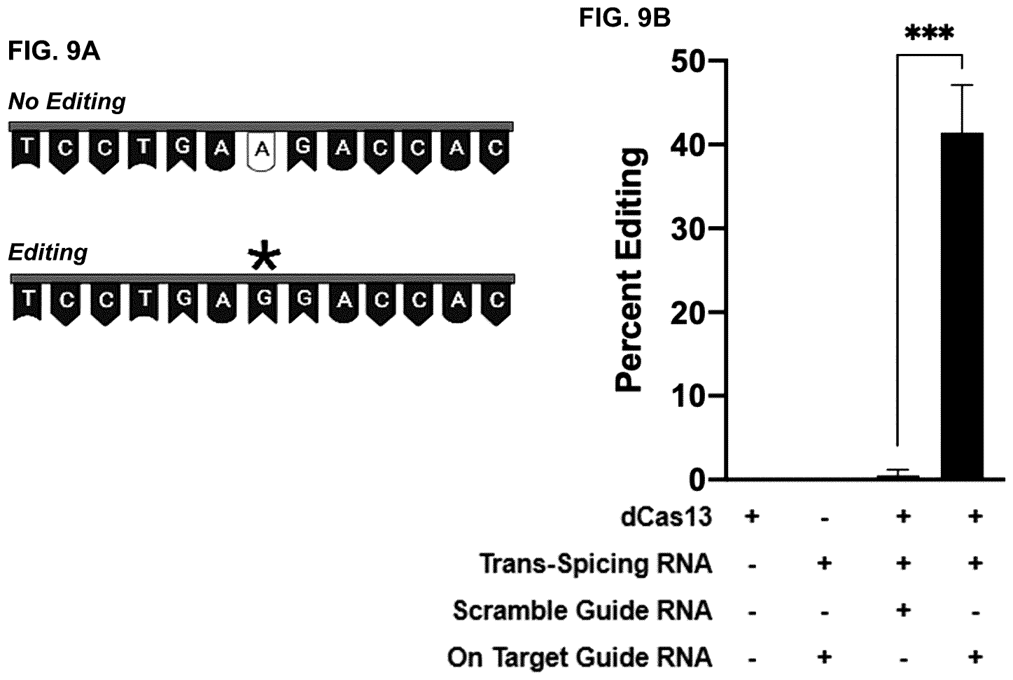

The efficiency of this editing strategy was measured by unbiased amplicon sequencing across the exon 74/75 splice junction. The percent of reads containing the encoded silent mutation correspond to the percent of transcripts that are trans-spliced (FIG. 9). Based on this editing strategy, the percent of trans-spliced reads was 41.33% when a dCasRx expression plasmid was transfected with the trans-splicing expression plasmid containing an on-target guide sequence (n=3) and was significantly greater than any other condition tested (p<0.0002).

| Name | Sequence |
|---|---|
| DMD 3′ RTM 2 | tccttgcttggtggcatctgtctgaagaaggcacattgtgagttagaagtgatcatgtgagaagactttttaa |
| | accaagggacctgtgacataataaatgtaatacagaactgaaagtccaatgatttgcattatcctgacacaa |
| | tacag (SEQ ID NO: 23) |
| DMD 3′ RTM 4 | aatcttactttgagatgaaagctatacctgcatttctgtcataagataaagagggagaaatatcttcccatgt |
| | gttgatgatacactttaaaatcacaatccaggagaggcctcaagcaacaactggcctttgcatctaactact |
| | tacaa (SEQ ID NO: 24) |
| DMD 3′ RTM 9 | atgtataagaatggagtaagtatacccatgttcctatcacctccccttgataagtttatcatttgccatattttct |
| | tcaaaagttttttgaagaaagaaaagttaacagccaaagcagaagctccctcacatgcccgcctcaacac |
| | cata (SEQ ID NO: 25) |

### Example 2

### CRISPR RNP Machine Increased Trans-Splicing Efficiency

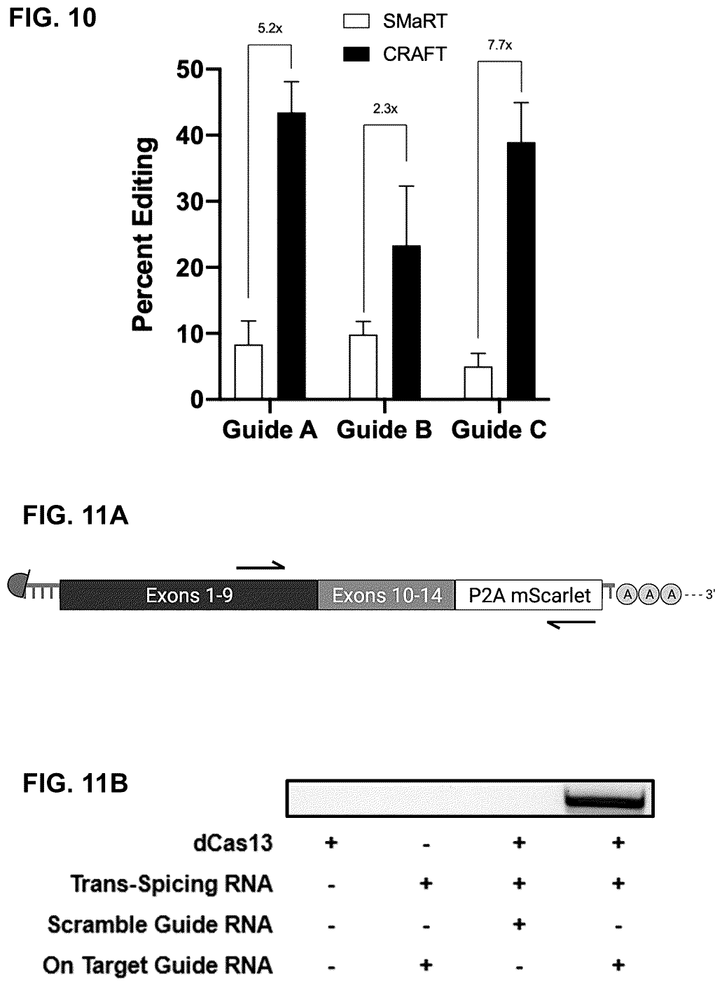

To demonstrate the benefit of guiding the trans-splicing machinery with a CRISPR RNP, the trans-splicing efficiencies of the technology disclosed was compared to a previous trans-splicing technology (i.e., SMART (Spliceosome Mediated RNA Trans-Splicing)). RNA editing efficiency was evaluated in the same manner as described in Example 1 (supra) and compared at three guide sequences along intron 74 or DP71. The 3 guide sequences were A, B, and C (FIG. 10). Across all three guide sequences an increase in trans-splicing efficiency is noted in with the present technology of a 5.2, 2.3 and 7.7-fold at guides A, B, and C respectively (n=3).

### Example 3

### Validation of 3′ Trans-Splicing in the DMPK Transcript

A PCR based validation of 3′ editing at the DMPK locus in HEK293 cells was developed. FIG. 11A depicts what a trans-spliced DMPK transcript would look like comprised of the endogenous 5′ exons of the transcript (black) the trans-spliced remaining exons (gray) and an additional mScarlett tag on the trans-spliced exons. RT-PCR across the splice junction was used as a primary endpoint of trans-splicing validation. Cells transfected with either the dCasRx (lane 1) or trans-splicing RNA (lane 2) expression plasmid did not yield detectable trans-splicing. Further, when the dCasRx expression plasmid was transfected with a trans-splicing RNA expression plasmid lacking a targeting sequence to the transcript of interest (lane 3), trans-splicing was not detected. FIG. 11B shows that only when dCasRx expression plasmid was transfected with the trans-splicing expression plasmid containing an on-target guide sequence, was trans-splicing detected by RT-PCR (lane 4). This band was confirmed to be the trans-spliced RNA by Sanger sequencing of the band to detect a silent G>T encoded uniquely in the trans-spliced RNA product (FIG. 12).

### Example 4

### RT-PCR Confirmed dCasRX Expression Generated Trans-Splicing

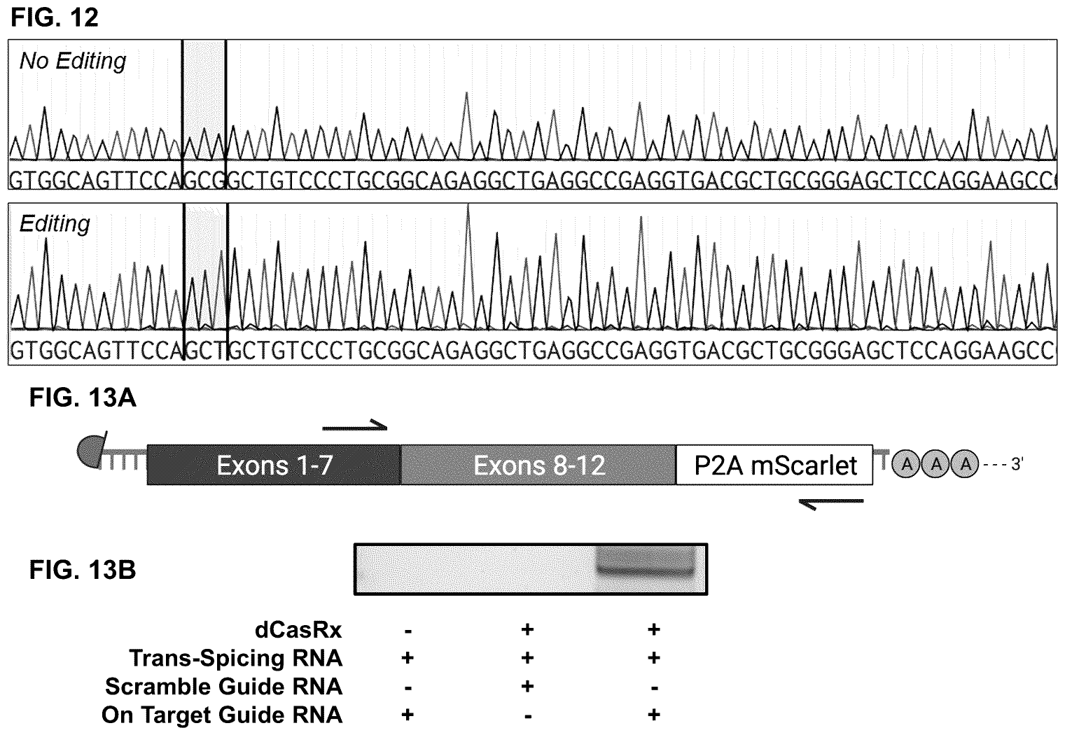

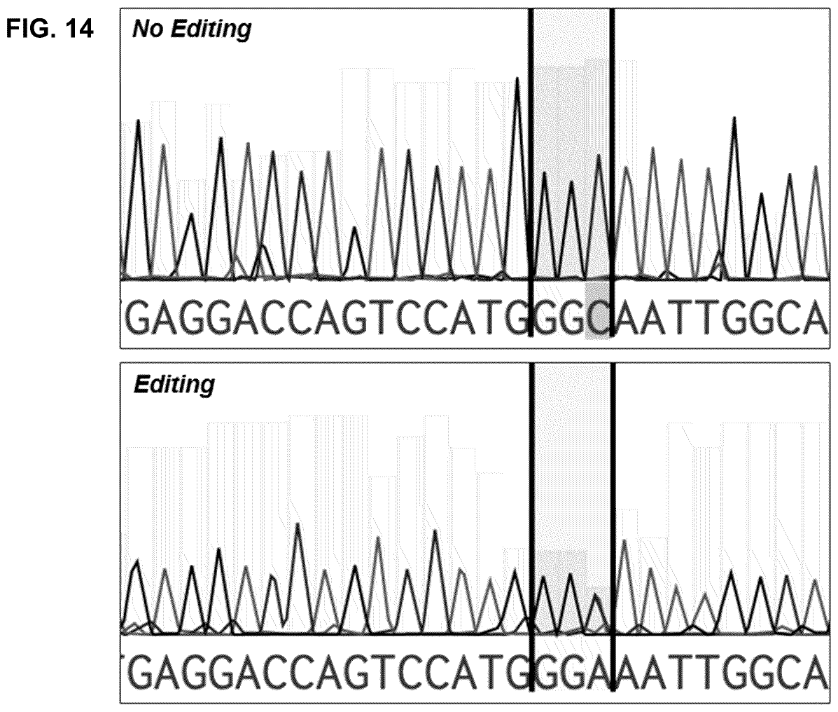

FIG. 13 depicts a PCR based validation of 3′ editing at the LMNA locus in HEK293 cells. FIG. 13 (top) depicts what a trans-spliced LMNA transcript would look like comprised of the endogenous 5′ exons of the transcript (black) the trans-spliced remaining exons (gray) and an additional mScarlett tag on the trans-spliced exons. RT-PCR across the splice junction was used as a primary endpoint of trans-splicing validation. Cells transfected with only the trans-splicing RNA (lane 1) expression plasmid did not yield detectable trans-splicing. Further, when the dCasRx expression plasmid was transfected with a trans-splicing RNA expression plasmid lacking a targeting sequence to the transcript of interest (lane 2), trans-splicing was not detected. Only when dCasRx expression plasmid was transfected with the trans-splicing expression plasmid containing an on-target guide sequence, was trans-splicing detected by RT-PCR (lane 3). This band is confirmed to be the trans-spliced RNA by Sanger sequencing of the band to detect a silent G>A encoded uniquely in the trans-spliced RNA product (FIG. 14).

### Example 5

### Editing Strategy Generated Full Replacement of 3′ Exons

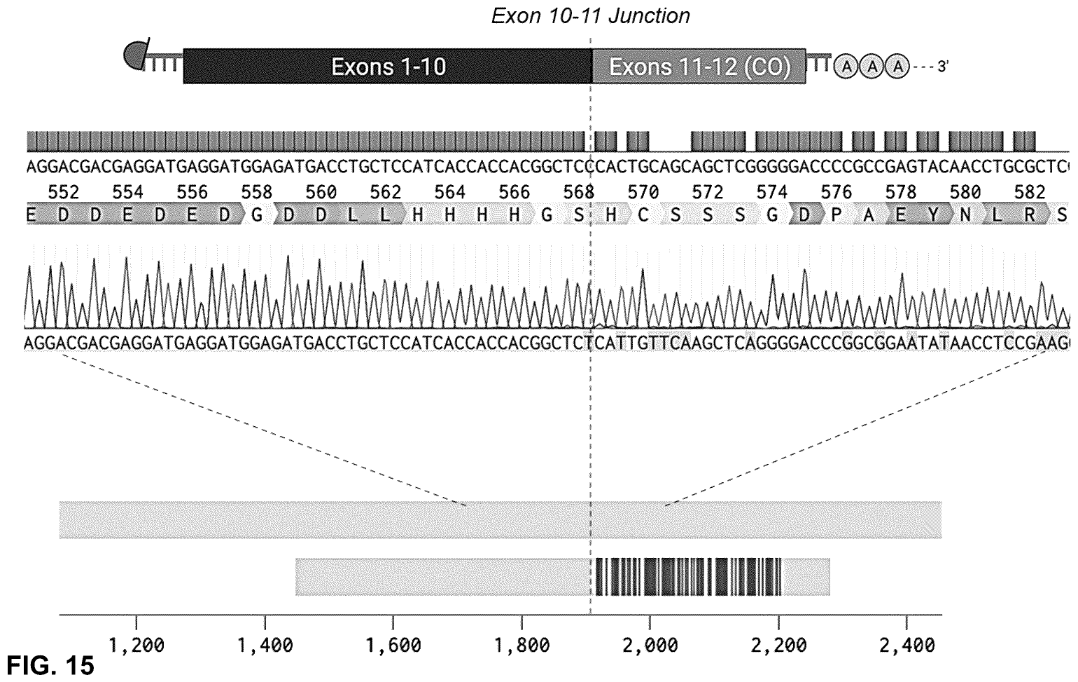

The final two exons of the human LMNA transcript were replaced with codon optimized sequenced using the proposed RNA editing machinery (FIG. 15).

### Example 6

### Validation of 5′ Trans-Splicing in the LMNA Transcript

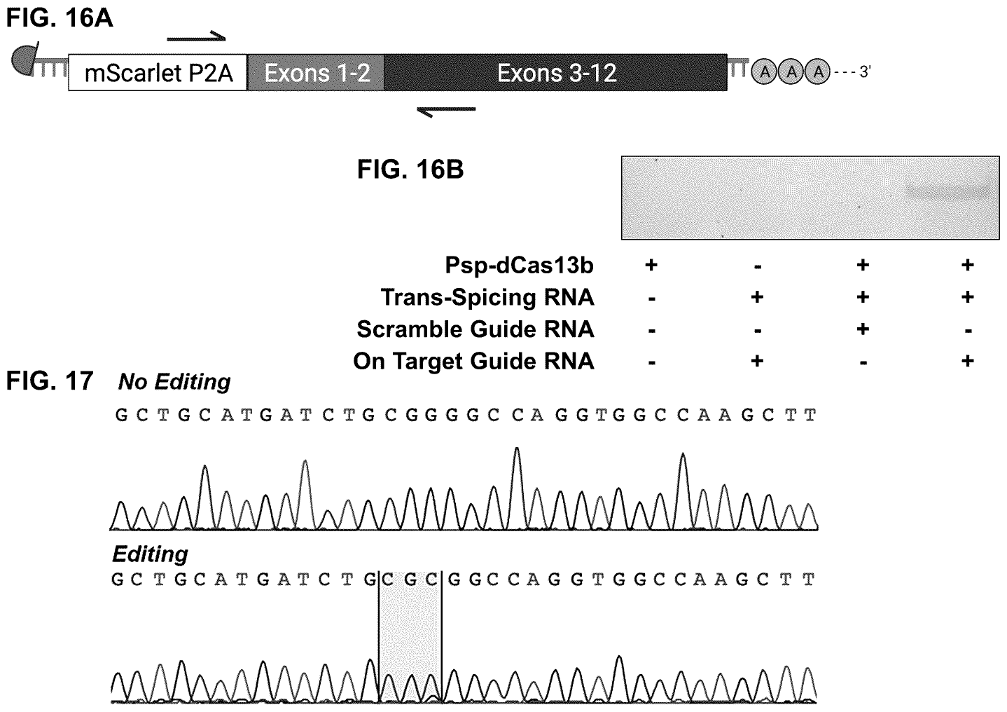

FIG. 16 depicts a PCR based validation of 5′ editing at the LMNA locus in HEK293 cells. FIG. 16 (top) depicts what a trans-spliced LMNA transcript would look like comprised of mScarlett tag (white) linked to the trans-spliced exons (gray) followed by the endogenous 3′ exons of the transcript (black). RT-PCR across the splice junction was used as a primary endpoint of trans-splicing validation. Cells transfected with either the dCas13b (lane 1) or trans-splicing RNA (lane 2) expression plasmid did not yield detectable trans-splicing. Further, when the dCas13b expression plasmid was transfected with a trans-splicing RNA expression plasmid lacking a targeting sequence to the transcript of interest (lane 3), trans-splicing was not detected. Only when dCas13b expression plasmid was transfected with the trans-splicing expression plasmid containing an on-target guide sequence, was trans-splicing detected by RT-PCR (lane 4). This band was confirmed to be the trans-spliced RNA by Sanger sequencing of the band to detect a silent G>C encoded uniquely in the trans-spliced RNA product (FIG. 17).

### Example 7

### Quantification of 3′ Trans-Splicing Efficiency at the DMPK Locus

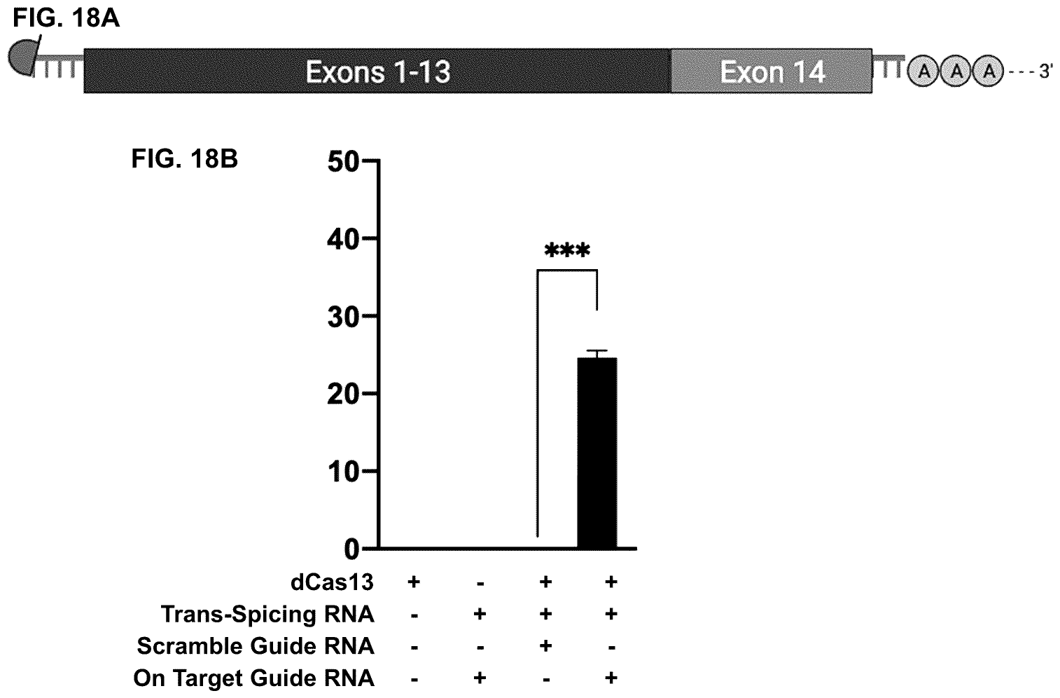

RNA editing efficiency of the proposed system at the DMPK locus in accordance with one embodiment of the present disclosure. A trans-splicing strategy was designed to replace exon 14 of the DMPK transcript such a recombinant exon 14 is joined to the endogenous exons 1-13. The efficiency of this editing strategy was measured by unbiased amplicon sequencing across the exon 13/14 splice junction. The percent of reads containing the encoded silent T>A (P593) mutation correspond to the percent of transcripts that are trans-spliced (FIG. 18). Based on this editing strategy the percent of trans-spliced reads is 24.65% when a dCasRx expression plasmid was transfected with the trans-splicing expression plasmid containing an on-target guide sequence (n=3) and was significantly greater than any other condition tested (p<0.0001).

### Example 8

### Quantification of 3′ Trans-Splicing Efficiency at the LMNA Locus

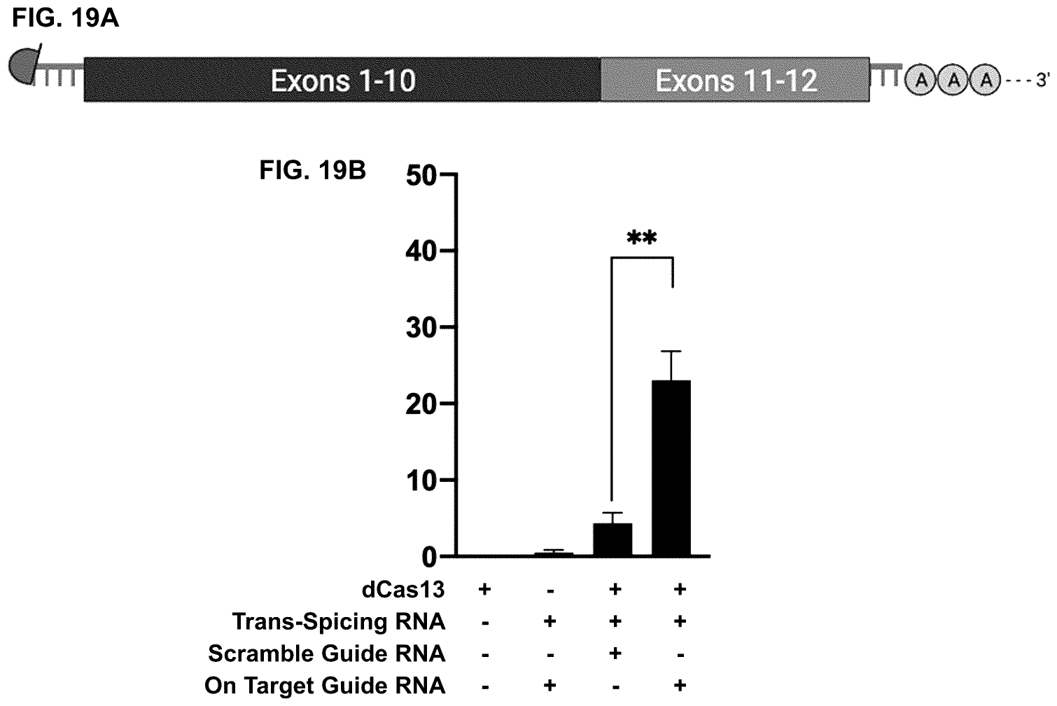

RNA editing efficiency at the LMNA locus using the methodology disclosed herein was explored. A trans-splicing strategy was designed to replace exons 11-12 of the LMNA transcript such recombinant exons 11-12 are joined to the endogenous exons 1-10. The efficiency of this editing strategy was measured by unbiased amplicon sequencing across the exon 10/11 splice junction. The percent of reads containing the encoded silent T>C (A577) mutation correspond to the percent of transcripts that were trans-spliced (FIG. 19). Based on this editing strategy, the percent of trans-spliced reads was 23.07% when a dCasRx expression plasmid was transfected with the trans-splicing expression plasmid containing an on-target guide sequence (n=3) and was significantly greater than any other condition tested (p≤0.0013).

### SUMMARY OF EXAMPLES

As demonstrated by the Examples, the compositions and methods disclosed herein are superior to previously disclosed compositions and methods. For at least three reasons, the data provided herein show that CRISPR Assisted Fragment Trans-Splicing (CRAFT) provides surprisingly exceptional results when compared to known technologies.

First, the nuclear localization signal on Cas13 promoted retention of the RNA editing machinery in the nucleus, where the target endogenous pre-mRNA existed. This represents an engineered improvement over other technologies such as Spliceosome Mediated RNA Trans-Splicing (SMART), which lacks a NLS, and therefore has a lower concentration of trans-splicing RNA within the nucleus where splicing occurs.

Second, the Cas enzyme stabilized the interaction of the guide RNA with the target endogenous pre-RNA molecule, both through optimal presentation of the guide sequence and a conformation change in the enzyme upon target recognition to stabilized RNA binding. The enhanced stability of this interaction promoted association of the trans-splicing RNA and target endogenous pre-mRNA and enhanced the efficiency of the tool due to the proximity of the splicing signals. Third, the CAS enzyme also inhibited cis splicing upon binding to a target endogenous RNA molecule. As the editing strategy is predicated on tipping the balance of splicing from cis to trans by reducing cis splicing, trans splicing rates can increase using this methodology.

1. An isolated nucleic acid molecule, comprising:
a nucleic acid sequence to be trans-spliced to a target endogenous pre-mRNA;
a 3′ hemi intron linked to the nucleic acid sequence to be trans-spliced or a 5′ hemi intron linked to the nucleic acid sequence to be trans-spliced;
one or more guide RNA sequences;
a promoter operably linked to the one or more guide RNA sequences; and
a nucleic acid sequence encoding an RNA binding protein.

2. The isolated nucleic acid molecule of claim 1, further comprising one or more stem loops.

3. The isolated nucleic acid molecule of claim 1, wherein the RNA binding protein has a bispecific affinity for the target endogenous pre-mRNA and a catalytically inactive Cas13.

4. The isolated nucleic acid molecule of claim 3, wherein the catalytically inactive Cas13 comprises RfxCas13d or PspdCas13b.

5. The isolated nucleic acid molecule of claim 1, wherein the one or more guide RNA sequences are directed to the intron immediately 3′ to the last exon of the target endogenous pre-mRNA.

6. The isolated nucleic acid molecule of claim 1, wherein the 3′ hemi intron is recognized by nuclear splicing components within a host cell.

7. The isolated nucleic acid molecule of claim 1, wherein the nucleic acid sequence to be trans-spliced encodes DP71, DMPK, or LMNA/C, or a portion thereof.

8.-11. (canceled)

12. The isolated nucleic acid molecule of claim 1, wherein the one or more guide RNA sequences are directed to the intron immediately 5′ to the first exon of the target endogenous pre-mRNA.

13. The isolated nucleic acid molecule of claim 1, wherein the 5′ hemi intron is recognized by nuclear splicing components within a host cell.

14.-21. (canceled)

22. An isolated nucleic acid molecule, comprising:
a nucleic acid sequence encoding a catalytically inactive PspdCas13b or a catalytically inactive RfxCas13d,
a promoter operably linked to the nucleic acid sequence encoding the catalytically inactive PspdCas13b or the nucleic acid sequence encoding the catalytically inactive RfxCas13d; and
a polyadenylation signal.

23. (canceled)

24. A transcriptome engineering system, comprising:
the isolated nucleic acid molecule of claim 4; and
the isolated nucleic acid molecule of claim 22.

25. The transcriptome engineering system of claim 24, wherein the isolated nucleic acid molecules form a ternary complex with the target endogenous pre-mRNA molecule, and wherein the resulting chimeric RNA molecule comprises the trans-spliced nucleic acid sequence.

26. A vector, comprising: the isolated nucleic acid molecule of claim 4.

27. A vector, comprising: the isolated nucleic acid molecule of claim 22.

28. (canceled)

29. A method of generating a chimeric RNA molecule in a cell, the method comprising:
contacting a target endogenous pre-mRNA in a cell with the isolated nucleic acid molecule of claim 4; and
contacting the target endogenous pre-mRNA in the cell with the isolated nucleic acid molecule of claim 22;
wherein the isolated nucleic acid molecules form a ternary complex with the target endogenous pre-mRNA molecule, and
wherein the resulting chimeric RNA molecule comprises the trans-spliced nucleic acid sequence.

30. The method of claim 29, wherein the cell is in a subject.

31. The method of claim 30, wherein the subject has been diagnosed with or is suspected of having a genetic disease or disorder.

32. A method of treating a genetic disease or disorder, the method comprising:
generating a chimeric RNA molecule in one or more cells by administering to a subject in need thereof a therapeutically effective amount of (i) the vector of claim 26 and (ii) a vector of claim 27;
wherein the resulting chimeric RNA molecule comprises the trans-spliced nucleic acid sequence; and
wherein the resulting chimeric RNA molecule restores one or more aspects of cellular homeostasis and/or cellular functionality and/or metabolic dysregulation.

33. The method of claim 32, wherein restoring one or more aspects of cellular homeostasis and/or cellular functionality and/or metabolic dysregulation comprises restoring the functionality and/or structural integrity of a missing, deficient, and/or mutant protein or enzyme.

34. The method of claim 32, wherein the therapeutically effective amount of the vector comprises about 1×10¹⁰ vg to about 2×10¹⁴ vg.

35.-44. (canceled)
